(19) United States
(12) Patent Application Publication
(10) Pub. No.: US 2015/0004133 A1
(43) Pub. Date: Jan. 1, 2015

(54) Compositions And Methods For Treating Steatohepatitis, Liver Fibrosis, and Hepatocellular Carcinoma (HCC)

(71) Applicant: The Regents of the University of California, Oakland, CA (US)

(72) Inventors: Tatiana Kisseleva, La Jolla, CA (US); David Brenner, La Jolla, CA (US)

(21) Appl. No.: 14/298,349

(22) Filed: Jun. 6, 2014

Related U.S. Application Data
(60) Provisional application No. 61/832,391, filed on Jun. 7, 2013.

(51) Int. Cl. C07K 16/24; C07K 16/18; A61K 38/20; C07K 16/40

(52) U.S. Cl. CPC C07K 16/244; C07K 16/40; C07K 16/18; A61K 38/20
USPC 424/85.2; 424/158.1; 424/145.1; 424/142.1; 424/133.1

(57) ABSTRACT

The invention provides methods and compositions for reducing symptoms of steatohepatitis and/or liver fibrosis and/or hepatocellular carcinoma (HCC) in a mammalian subject in need thereof, comprising administering to the mammalian subject a therapeutic amount of a compound that reduces the level of interleukin 17 (IL-17) and/or interleukin 23 (IL-23) and/or signal transducer and activator of transcription 3 (Stat3) and/or Janus kinase 2 (Jak2). The invention's methods may comprise administering to the mammalian subject a therapeutic amount of a compound that increase the level of interleukin 22 (IL-22) and/or interleukin 25 (IL-25) and/or interleukin 27 (IL-27). The invention's methods may comprise administering to the mammalian subject a therapeutic amount of interleukin 22 (IL-22) and/or interleukin 25 (IL-25) and/or interleukin 27 (IL-27).

This application claims priority under 35 U.S.C. §119(e) to co-pending to U.S. Provisional Application Ser. No. 61/832,391, filed on Jun. 7, 2013, herein incorporated by reference in its entirety.

### GOVERNMENT INTEREST

This invention was made with government support under grant numbers GM41804, AA15055, DK72237, and AI077780, awarded by the National Institutes of Health (NIH). The government has certain rights in the invention.

### BACKGROUND

Fibrosis of the liver is the outcome of many chronic liver diseases, including hepatitis B virus (HBV), hepatitis C virus (HCV), alcoholic liver disease and non-alcoholic steatohepatitis (NASH). It is manifested by massive accumulation of extracellular matrix (ECM) and scar formation and often progresses to hepatocellular carcinoma.

Steatohepatitis (fatty liver disease) is classically seen in alcoholics as part of alcoholic liver disease, and also is frequently found in people with diabetes. Steatohepatitis may progress to cirrhosis, a late stage of hepatic fibrosis.

Currently, there are no optimal treatments for liver fibrosis or HCC. Thus, there remains a need for methods and compositions that reduce symptoms of steatohepatitis, liver fibrosis, and hepatocellular carcinoma.

### SUMMARY OF THE INVENTION

The invention provides a method for reducing one or more symptoms of one or more of steatohepatitis, liver fibrosis, and hepatocellular carcinoma (HCC) in a mammalian subject in need thereof, comprising administering to said mammalian subject a therapeutic amount of one or more compounds that reduce the level of one or more of

(a) interleukin 17 (IL-17),

(b) interleukin 23 (IL-23),

(c) signal transducer and activator of transcription 3 (Stat3), and

(d) Janus kinase 2 (Jak2),

in one or more of hepatic stellate cells (HSCs), liver resident Kupffer cells (KCs), immune cells, and inflammatory cells, wherein said therapeutic amount reduces said one or more symptoms. Data herein demonstrates the role of IL-17 in alcoholic steatohepatitis (Example 16), liver fibrosis induced by cholestatic injury or by hepatotoxic injury (Examples 2-14), or alcohol induced liver fibrosis (Example 17). In one embodiment, the steatohepatitis is alcoholic steatohepatitis (exemplified in Example 16). In another embodiment, the liver fibrosis is alcoholic liver fibrosis (exemplified in Example 17). In a further embodiment, the liver fibrosis is cholestatic liver fibrosis (exemplified in Examples 2-14). In yet another embodiment, the liver fibrosis is hepatotoxic liver fibrosis (exemplified in Examples 2-14). In another embodiment, the one or more compounds is an antibody, or an antigen-binding fragment thereof, that specifically binds to said one or more of interleukin 17 (IL-17), interleukin 23 (IL-23), Stat3, and Jak2. In a particular embodiment, the antibody is a monoclonal antibody. In a particularly preferred embodiment, the monoclonal antibody is a human antibody. In one embodiment, the human monoclonal antibody specifically binds to IL-17 and comprises human monoclonal antibody AIN457. In another embodiment, the antibody is a humanized antibody.

The invention also provides a method for reducing one or more symptoms of one or more of steatohepatitis and liver fibrosis in a mammalian subject in need thereof, comprising administering to said mammalian subject a therapeutic amount of one or more compounds that increase the level of one or more of

(a) interleukin 25 (IL-25, also called IL-17E, (exemplified in Examples 12), and

(b) interleukin 27 (IL-27),

in one or more of immune cells, inflammatory cells, and liver resident fibrogenic myofibroblasts (Hepatic stellate cells), wherein said therapeutic amount reduces said one or more symptoms. This can include synthetic cytokines or agonists of IL-25- and/or IL-22- and/or IL-27-binding receptors.

Also provided by the invention is a method for reducing one or more symptoms of one or more of steatohepatitis and liver fibrosis in a mammalian subject in need thereof, comprising administering to said mammalian subject a therapeutic amount of one or more of

(a) interleukin 25 (IL-25) (exemplified in Examples 12), and

(b) interleukin 27 (IL-27),

wherein said therapeutic amount reduces said one or more symptoms.

### BRIEF DESCRIPTION OF THE DRAWINGS

FIG. 1. A. The paradigm of Th17 signaling in response to ALD (explanation in the text). B. Liver biopsy specimens were obtained during routine diagnostic. Alcoholic liver lesions were assessed, and fibrosis stages were defined as F0 (n=2), F1 (n=5), F2 (n=5), F3 (n=4). mRNA was isolated from snap frozen tissues. Expression of IL-17RA was determined by qRT-PCR with primers for hIL-17RA, and normalized to GAPDH. The data are means±SEM *p<0.03, **p<0.01 versus F0.

FIG. 2. IL-17 signaling is induced in mice with alcoholic steatohepatitis. A. Chronic-binge model recruits MPO′ neutrophils to the liver. Liver sections were stained with H&E, and for MPO. The number of neutrophils per ×100 field was counted. Micrographs are shown using ×40 objective. B. Comparison of liver injury and inflammation induced by chronic, binge, or chronic-binge ethanol feeding. Serum ALT and AST levels are measured. The liver mRNA levels of cytokines and chemokines were analyzed by real-time quantitative PCR, the data are fold mRNA induction. C. Increased hepatic expression of IL-17 and IL-17 regulating cytokines (but not fibrogenic genes) was observed in chronic-binge mice. The data shown represent the means±SEM. *p<0.05, **p<0.01, ***p<0.001 for EtOH-fed vs pair-fed.

FIG. 3. IL-17 signaling is induced in mice in response to intragastric ethanol model of alcoholic fibrosis. A. Intragastric ethanol induces steatohepatitis and liver fibrosis. Micrographs of H&E and Sirius Red staining, and staining with anti-IL-17 Ab are shown using ×40 objective. B. Liver fibrosis was induced in alcohol-fed mice (vs pair-fed mice), as shown by increased mRNA expression of fibrogenic genes using quantitative RT-PCR, the data are fold mRNA induction, represent the means±SEM. *p<0.05, **p<0.01, for EtOH-fed vs pair-fed.

FIG. 4. Alcoholic liver fibrosis is reduced in IL-17RA^{-/-} mice. ALT and AST (A), steatosis (H&E, triglycerides) B), liver fibrosis (Sirius Red), the number of activated α-SMA ⁺HSCs and F4/80⁺ Kupffer cells, lipid peroxidation (4-HNE), CYP2E1 protein (C) were reduced in alcohol-fed IL-17RA^{-/-} mice. qRT-RCR shows mRNA expression of fibrogenic and myeloid markers (D), cytokines (E), lipogenic genes (F), and ROS production (G) in alcohol-fed IL-17RA^{-/-} and wt mice. *p<0.01, **p<0.05.

FIG. 5. IL-17 signaling is induced in mice in response to DEN-alcohol model of HCC. A. Gross liver images from DEN- and DEN-alcohol-treated mice (at 48 weeks of age). B. Development of liver fibrosis in alcohol-fed (at 24 w of age), and DEN-alcohol-treated mice (at 24 and 48 w of age), H&E and Sirius Red staining are shown. Immunostaining with anti-IL-17 Ab demonstrated an association of IL-17-expressing cells with HCCs in DEN-alcohol-treated mice (at 48 w of age). Micrographs are shown using ×20 objective. C. Expression of fibrogenic and inflammatory genes in control (pair-fed), alcohol-fed, DEN- and DEN-alcohol-treated mice was analyzed by qRT-PCR. The data represent the means±SEM. *p<0.05, **p<0.01, for DEN- vs DEN-alcohol-treated mice.

FIG. 6. Development of liver fibrosis is strongly inhibited in IL-17RA^{-/-} mice. (A) Expression of IL-17A and IL-17F, IL-17RA and IL-17C genes is upregulated during liver fibrosis. The data is fold induction of mRNA expression from whole liver of BDL (n=10) and CCl₄-treated (n=10) mice compared to control mice, *p<0.01. Fibrogenic liver injury is associated with increased serum IL-17A detected in BDL (5 days, n=6; 10 days n=5) and CCl₄-treated (8 weeks, n=5) mice by ELISA, ^{**}p<0.05. (B) Development of BDL- and CCl₄-induced liver fibrosis is inhibited in IL-17RA^{-/-} mice. Livers from wild type mice (untreated n=3), BDL (n=10) and CCl₄-treated (n=10), and IL-17RA^{-/-} mice (untreated n=3), BDL (n=9) and CCl₄-treated (n=10), were analyzed by H&E, Sirius Red and α-SMA staining and quantified (*p<0.01). Bright field micrographs are shown using ×10 objectives. Liver function was assessed by ALT, *p<0.01. (C) Expression of ibrogenic genes is downregulated in whole livers from BDL and CCl₄-treated IL-17RA^{-/-} mice compared to wild type mice. The data are shown as fold mRNA induction compared with untreated mice, *p<0.02. (D) Reduced expression of α-SMA protein was detected in livers of BDL- and CCl₄-treated IL-17RA^{-/-} mice compared to the wild type mice.

FIG. 7. IL-17A/IL-17RA signaling in fibrotic liver is mediated by immune cells, KC and HSCs. (A) Hepatic T cells are the major source of IL-17A in fibrotic livers. Lymphoid fraction from sham (n=3) and BDL (n=3) wild type mice were in vitro stimulated with PMA (100 ng/ml)+Ionomycin (500 ng/ml) or IL-23 (20 ng/ml) for 5 hours, and analyzed by flow cytometry for intracellular IL-17A and IL-17F expression. Expression of IL-17A was further analyzed in CD45⁺, CD3⁺, CD4⁺, CD8a⁺, TCRγδ⁺, TCRβ⁺, NK1.1⁺, CD11b⁺ cells. Representative dot plots are shown. (B)Primary wild type hepatocytes, quiescent and in vitro activated HSCs, and KC were stimulated with IL-17A (10 ng/ml), IL-17F (10 ng/ml), or a combination of both. mRNA levels of IL-17 cytokine/receptors and inflammatory gene expression are shown as fold induction compared with untreated cells, *p<0.01, ** non significant.

FIG. 8. Selective ablation of IL-17 signaling in inflammatory cells+KC and liver resident cells results in inhibition of BDL-induced liver fibrosis. (A) Development of liver fibrosis in BM chimeric mice was compared. Livers from BDL-injured Wt→wt (n=7), IL-17A^{-/-}→wt (n=10), Wt→IL-17RA^{-/-} (n=10), IL-17RA^{-/-}→wt (n=10), and IL-17RA^{-/-}→IL-17RA^{-/-} (n=8) mice were analyzed by H&E, Sirius Red staining, and α-SMA immunohistochemistry. Representative bright field micrographs are shown using ×10 objectives. (B) Quantification of Sirius Red staining and α-SMA immunohistochemistry in BM chimeric mice. Results are shown as fold induction compared with the sham-control Wt→wt mice. (Each group of BMT mice is compared to the BDL-injured wt mice, p<0.01. For Wt→IL-17RA mice, p<0.05). (C) Fibrogenic mRNAs are downregulated in whole liver from BDL-operated IL-17A^{-/-}→wt, Wt→IL-17RA^{-/-}, or IL-17RA^{-/-}→wt mice, compared to BDL-operated Wt→wt mice (p<0.01). The data are shown as fold induction compared with sham Wt→wt mice. (D) Expression of α-SMA protein, evaluated by Western blot, was progressively downregulated in Wt→IL-17RA^{-/-}>IL-17A^{-/-}→wt>IL-17RA^{-/-}→wt>IL-17RA^{-/-}→IL-17RA^{-/-} mice. Representative images are shown.

FIG. 9. IL-17A facilitates HSC activation into myofibroblasts via Stat3. (A) IL-17A induced nuclear translocation of p65 and Stat3 in primary murine HSCs. HSCs (5×10⁵ cells) were stimulated with IL-17A (10 ng/ml), TNF-α (20 ng/ml) or IL-6 (30 ng/ml) for 0-30 min, translocation of p65 (NF-κB) or Stat3 was analyzed by fluorescent microscopy using ×40 objective. (B) IL-17A stimulates collagen-α1(I) expression by HSCs. Primary murine qHSCs were isolated from Col-GFP or Col-GFP^{IL-17RA-/-} mice, and stimulated with IL-17A (10 ng/ml) or TGF-β1 (3 ng/ml) in 1% FSCs for 48 hours. The percent of cells upregulated collagen-α1(I)-GFP was calculated, p<0.05. Representative images are shown using ×20 objective. (C) Fibrogenic properties of IL-17A, IL-6 and leptin. Fibrogenic mRNA expression was tested in HSCs treated with IL-17A (10 ng/ml), IL-6 (30 ng/ml), or leptin (100 ng/ml) for 4 h. IL-17A induced production of IL-6 in HSCs. The direct effect of IL-17A on collagen-α1(I) mRNA expression was assessed in IL-17A-stimulated HSC cultures depleted for IL-6 (using anti-IL-6 antibody; 0.5 μg/ml). *p<0.01, **p<0.05, ns non significant. (D) IL-17A activates Stat3 in HSCs. Deletion of Stat3 in HSCs was achieved by crossing GFAP-Cre mice×floxed Stat3^{f/f} mice=GFAP^{Stat3-/-} mice. 1 qHSCs were isolated from GFAP^{Stat3-/-} or Stat3^{f/f} wt mice, and stimulated with IL-17A (10 ng/ml) or TGF-β1 (3 ng/ml) for 15 min. Nuclear translocation of Stat3 (but not phosphor-Smad2/3) was detected in wild type HSCs but not in Stat3-deficient HSCs. Representative images are shown using ×20 objective. (E) IL-17-dependent collagen-α1(I) expression is impaired in Stat3-deficient HSCs. Primary wild type (Stat3^{f/f}) and Stat3-deficient HSCs from GFAP^{Stat3-/-} mice were cultured in DMEM+10% FCS for 18 h and stimulated with IL-17A (10 ng/ml in 1% FSCs) for 8 h, and mRNA levels of collagen-α1(I) expression were measured, *p<0.01.

FIG. 10. Stat3-deficient HSCs have decreased activation. (A) Upregulation of Stat3, IL-17RA, IL-6RA, OSM-R, and LIF-1-R, but not Stat1 or IL-10Ra/b, was detected in aHSCs in comparison with qHSCs using the whole mouse genome microarray (Suppl. Methods). The mRNA level is the average of the multiple probes (p<0.01). (B) Development of liver fibrosis is attenuated in GFAP^{Stat3-/-} mice compared to wild type Stat3^{f/f} mice. Livers from wild type mice (untreated (n=2), BDL (n=4) and CCl₄-treated (n=4)), and GFAP^{Stat3-/-} mice (untreated (n=2), BDL (n=5) and CCl₄-injured (n=6)), were analyzed by Sirius Red staining, and immunostaining for α-SMA and Desmin, and quantified (as percent of positive area, *p<0.01, **p<0.05). Representative bright field micrographs are shown using ×10 objectives. (C) Stat3-deficient and wild type primary HSCs were in vitro stimulated with IL-17A (10 ng/ml, 4 h). The ability to activate α-SMA and Collal mRNA, and produce cytokines HGF, TGF-β1, PDGF, IL-6 mRNA was impaired in Stat3^{-/-} HSCs compared to wt HSCs. *p<0.001, **p<0.005, ns—non significant.

FIG. 11. IL-23 and IL-22 have opposing roles in liver fibrosis. (A) Serum IL-17A, IL-17E (IL-25), IL-17F, IL-6 and IL-22 were measured by ELISA in BDL (10 days) IL-17RA^{-/-} mice versus wild type mice (*p<0.01, **p<0.05). (B) Hepatic levels of IL-17A, IL-17F, IL-23 and IL-22 mRNA were detected in BDL (10 days) IL-17RA^{-/-} mice versus wild type mice (p<0.05). (C) Livers from BM chimeric CCl₄-treated Wt→wt (n=5), IL-17A^{-/-}→wt (n=6), IL-22^{-/-}→wt (n=6), and IL-23^{/--}→wt (n=6) mice were analyzed by Sirius Red, α-SMA staining Bright field micrographs are shown using ×10 objectives. Quantification of Sirius Red and α-SMA staining, mRNA expression is shown (p<0.05, compared to BDL wt mice). mRNA expression is fold induction compared to oil-control Wt→wt mice. (D) The percent of inhibition of liver fibrosis was calculated for BM chimeric mice in response to liver injury (in comparison Wt→wt mice, 100%, p<0.01) based on Sirius Red and α-SMA expression. The strongest inhibition of liver fibrosis was observed in IL-17RA^{-/-}→IL-17RA^{-/-} mice, the least inhibition of liver fibrosis was observed in Wt→IL-17RA^{-/-} mice. Increased fibrosis was detected in IL-22^{-/-}→wt mice. (E) Administration of IL-22 and IL-17E attenuates liver fibrosis. BDL-operated mice were daily treated with IL-22 (0.5 μg/mouse, n=4), IL-17E (0.5 μg/mouse n=5), or vehicle (PBS) for 7 days, tissues were analyzed by Sirius Red, and α-SMA staining; mRNA expression is shown (p<0.05, compared to BDL wt mice+PBS).

FIG. 12. Upregulation of IL-17A levels in patients liver fibrosis and cirrhosis. (A) Human liver tissues were obtained from patients with hepatitis C, diagnosed with clinical and pathological stages of liver fibrosis (F1) and cirrhosis (F4), or no fibrosis (F0), and analyzed by immunohistochemistry for expression of human IL-17A and Sirius Red staining Representative images are shown using ×4, 10 and 20 objectives. (B) Liver biopsy specimens were obtained from alcoholic patients diagnosed with F0 (n=2); F1 (n=5); F2 (n=5); F3 (n=4) stages of liver cirrhosis. mRNA was isolated from frozen liver biopsy tissues and analyzed by RT-PCR for IL-17RA expression.

FIG. 13. Intracellular IL-17A expression was detected in BDL-injured wild type but not IL-17A−/− mice. Lymphoid fraction from BDL-injured (n=3) wild type mice were in vitro stimulated with PMA (100 ng/ml)+Ionomycin (500 ng/ml) for 5 h, and analyzed by flow cytometry for intracellular IL-17A expression. IL-17A expression was detected in hematopoietic cells stained positive for lineage markers (B220, F4/80, CDR, CD11b, TCRb), and T cell marker CD90.2. Representative dot plots are shown.

FIG. 14. IL-17A induces IL-17A and IL-1β1 production in peritoneal macrophages. Peritoneal macrophages were isolated from thioglycollate-treated mice (6 days), cultured for 48 h in DMEM+10% FCS, then transferred to 1% FSC for 1 h and stimulated with IL-17A (10 ng/ml) for 6 h. Cell lysates were analyzed by ELISA (eBioscience), *p<0.05.

FIG. 15. Endothelial Cells (EC) express IL-17RA but do not respond to IL-17A stimulation. Primary wild type EC were in vitro stimulated with IL-17A (10 ng/ml), IL-17F (10 ng/ml), or combination of both. mRNA levels of IL-17A and IL-17RA are shown as fold induction compared with untreated cells, p value is non significant (ns).

FIG. 16. IL-17 signaling is abrogated in IL-17RA^{-/-} HSCs and Kupffer cells. Primary HSCs and Kupffer cells were isolated from wild type of IL-17RA^{-/-} mice and stimulated with IL-17A. The lack of response in IL-17RA^{-/-} cells indicates that 1) IL-17A regulates IL-17RA expression, 2) that deletion of IL-17RA^{-/-} in these cells makes them insusceptible to IL-17A signaling. The data are shown as fold mRNA induction compared with untreated HSCs, *p<0.02.

FIG. 17. Generation of BM chimeric mice. (A) Recipient mice were intravenously injected with liposomal clodronate (150 μl) to deplete Kupffer cells. BMT was performed 24 h later by transplantation of the whole BM into lethally irradiated (1200 Rad) recipient mice. (B) The efficiency of reconstitution BM was analyzed by flow cytometry in the wild type recipient mice transplanted with the β-actin-RFP BM. 90±2% of RFP⁺ cells were detected in the peripheral blood on chimeric β-actin-RFP→wt mice (vs 92±3% of RFP⁺ cells in β-actin-RFP mice), indicating that 97.8% of BM reconstitution was achieved. Representative dot plots are shown. (C) The efficiency of Kupffer cell reconstitution was analyzed by immunostaining for F4/80 of the clodronate-pretreated lethally irradiated wild type recipient mice transplanted with the β-actin-RFP BM. 92±4% of Kupffer cell reconstitution was achieved. Representative images are shown using ×40 objective.

FIG. 18. IL-17A activates Stat3 and NF-κB in KC and HSCs. (A) Primary murine KC and HSCs were isolated from BDL and CCl₄-treated IL-17RA^{-/-}→wt mice. Their activation was accessed by expression of inflammatory genes by quantitative RT-PCR, *p<0.01. (B) IL-17A induces TGF-β1 production in KC and peritoneal macrophages. Primary KC (from untreated wild type mice) and peritoneal macrophages (from thioglycollate-treated mice) were cultured for 48 h in DMEM+10% FCS, then transferred to 1% FSC for 1 h and stimulated with IL-17A (10 ng/ml) for an additional 6 h. Supernatants (triplicates) were analyzed by ELISA for activated TGF-β1, *p<0.05, **p<0.01. (C) IL-17A produced by KC (KC) stimulates wild type but not IL-17RA^{-/-} HSCs. Wild type or IL-17RA-deficient KC were stimulated with IL-17A for 6 h, washed and co-cultured with the Col-GFP or Col-GFP^{IL-17RA-/-} HSCs. The number of activated HSCs was estimated by fluorescent microscopy 24 hr after co-culture, and shown as a percent of collagen-α1(I)-GFP⁺ cells using ×20 objective. Representative images are shown, p<0.05 compared with co-culture of wt:wt cells. (D) Primary murine HSCs were isolated from BDL and CCl₄-treated Wt→IL-17RA^{-/-} mice. Their activation was accessed by expression of fibrogenic genes by quantitative RT-PCR, *p<0.01; #p<0.05. (E) Phosphorylation of Stat3, ERK, JNK, AKT was analyzed in IL-17A- (10 ng/ml) stimulated KC (5×10⁵ cells) and HSCs (5×10⁵ cells). The protein level was controlled for each sample by α-tubulin expression. (F) KC activate multiple signaling pathways in response to IL-17A stimulation. Primary KC were isolated from non-injured wild type mice, cultured for 24 h, then stimulated with IL-17A (10 ng/ml) and IL-17F (10 ng/ml)±inhibitors. U0126 (MEK1/2 inhibitor; (2 uM), EMD), AZD1480 (Jak1/2 inhibitor; (0.5 uM) XX), SB (p38 inhibitor, (2 uM), EMD). Cells were pre-incubated with inhibitors for 20 minutes prior to stimulation with IL-17A+IL-17F for 2 h. Expression of fibrogenic and pro-inflammatory cytokines was detected in KC by RT-PCR. *p<0.05 and ns−p>0.05, calculated in comparison with KC stimulated with IL17A+F.

FIG. 19. IL-17 signaling activates HSCs. (A) Schematic representation of the effects of IL-17A signaling on liver fibrogenesis in response to injury. BM-derived cells are the major source of IL-17A. IL-17A regulates production of TGF-β1 by BM-derived macrophages and Kupffer cells, which in turn, stimulate activation of HSC into fibrogenic myofibroblasts via Smad2/3 signaling pathway and further facilitate differentiation of IL-17A-producing cells. In addition, IL-17A stimulates collagen-α1(I) production in HSCs via Stat3 signaling pathway. (B) IL-17 signaling induces activation of LX-2 human HSC cell line IL-17A (10 ng/ml, 72 h) or IL-17F (10 ng/ml, 72 h) induce mRNAs of fibrogenic genes compared with non-stimulated cells, p<0.05. (C) IL-17 signaling induces activation of hTERT human HSC cell line. IL-17A (10 ng/ml, 8 h) and IL-17F (10 ng/ml, 8 h) induce mRNAs of fibrogenic and inflammatory cytokines compared with non-stimulated cells, detected by RT-PCR, p<0.03. Human IL-17A induced nuclear translocation of Stat3 in hTERT cells was observed 30 min after stimulation, representative images are taken using ×20 objective.

FIG. 20. Alcoholic steatohepatitis is reduced in IL-17RA^{-/-} mice. ALT and AST (A) were significantly reduced in IL-17RA−/− mice, which the liver/body weight was not changed. B. Steatosis (H&E, triglycerides) was not significantly changed. Meanwhile, liver fibrosis (Sirius Red), the number of activated α-SMA ⁺HSCs and F4/80⁺ Kupffer cells, lipid peroxidation (4-HNE), CYP2E1 protein (C) were reduced in alcohol-fed IL-17RA^{-/-} mice. qRT-RCR shows mRNA expression of fibrogenic and myeloid markers (D), cytokines, and ROS production in alcohol-fed IL-17RA^{-/-} and wt mice. *p<0.01, **p<0.05

### DEFINITIONS

To facilitate understanding of the invention, a number of terms are defined below.

“Alcoholic liver disease” (“ALD”) is a major cause of cirrhosis and liver failure, which is the 12th leading cause of death in adult patients in the United States¹. ALD progresses from steatosis, to steatohepatitis, fibrosis, cirrhosis, and finally hepatocellular carcinoma (HCC)¹⁻³. Several injury-triggered events (see below) play a critical role in the pathogenesis of ALD. There is no effective treatment of ALD, in part because there are no pre-clinical models available to study ALD progression.

“Steatohepatitis,” “liver steatosis” and “Fatty liver disease,” interchangeably refer to a disease which is characterized by the accumulation of fat droplets (mainly triglycerides and phospholipids) in hepatocytes.

“Fatty liver disease”/“steatohepatitis” includes “nonalcoholic Fatty liver disease” (also known as “nonalcoholic steatohepatitis” (“NASH”)), which is frequently found in people with diabetes and obesity and is related to metabolic syndrome.

“Fatty liver disease”/“steatohepatitis” also includes “alcoholic fatty liver disease” (also known as “alcoholic steatohepatitis” (“ASH”)), which results from a direct (via acetaldehyde) or indirect (via up-regulation of cytochrome P4502E1) effect of consumed ethanol on up-regulation of SREBP-1c and down-regulation of PPAR-α expression, leading to the induction of fatty acid synthesis and inhibition of β-oxidation^{1,4}. In one embodiment, development of fatty liver disease is regulated at the level of de novo lipid synthesis, lipid secretion (VLDL), and lipid beta oxidation. “Alcoholic steatohepatitis” (“ASH”) is manifested by steatosis, centrilobular ballooning of hepatocytes, neutrophilic infiltration, and MalloryDenk hyaline inclusions^{5,6}. A) In response to chronic alcohol, lipid peroxidation and mitochondrial damage causes apoptosis of hepatocytes, which trigger recruitment of inflammatory cells to the fatty liver, activation of bone marrow (BM) and resident macrophages (Kupffer cells) and release of pro-inflammatory cytokines (TGF-β1, IL-6, IL-1β, TNF-α). B) Infiltration of liver by neutrophils, which kill sensitized hepatocytes and further exacerbate alcohol-induced liver injury, is a prominent feature of ASH. Up-regulation of IL-8, CXCL1 (Gro-α), and IL-17 in the liver contributes to neutrophil infiltration and correlates with the severity of ASH⁷⁻⁹. IL-17 not only acts as a neutrophil chemoattractant, but also stimulates hepatic stellate cells (HSGs) to produce IL-8 and CXCL1 to facilitate neutrophil recruitment^{10,11}. A rodent model of alcoholic steatohepatitis has demonstrated a pivotal role of neutrophils in pathogenesis of ALD¹². C) Besides neutrophils, T and B lymphocytes are recruited to the damaged liver; they mediate adaptive immune response and contribute to alcohol-induced liver damage and myofibroblast activation.

“Liver fibrosis” and “hepatic fibrosis” interchangeably refer to the excessive accumulation of extracellular matrix proteins including collagen that occurs in most types of chronic liver diseases. (Bataller & Brenner. Liver fibrosis. J Clin Invest 2005; 115:209-18), including hepatitis B virus (HBV), hepatitis C virus (HCV), alcoholic liver disease and non-alcoholic steatohepatitis (NASH) (Bataller & Brenner 2005). Liver fibrosis is also manifested by scar formation. “Cirrhosis” is a late stage of hepatic fibrosis that has resulted in widespread distortion of normal hepatic architecture. Cirrhosis is characterized by regenerative nodules surrounded by dense fibrotic tissue. Symptoms may not develop for years and are often nonspecific (e.g., anorexia, fatigue, weight loss). Late manifestations include portal hypertension, ascites, and, when decompensation occurs, liver failure. Diagnosis often requires liver biopsy. Cirrhosis is usually considered irreversible.

Liver fibrosis includes “cholestatic liver fibrosis” that results from an impairment of bile formation or bile flow to the gallbladder and duodenum (first section of the small intestine) (EASL Clinical Practice Guidelines: Management of cholestatic liver diseases. J Hepatol. 2009; 51:237-267).

Liver fibrosis also includes “hepatotoxic liver fibrosis” which is caused by hepatotoxic drugs such as CCl4 (Jang et al. (2008) Transplant Proc. 40(8):2700-3).

Liver fibrosis further includes alcoholic liver fibrosis. “Alcoholic liver fibrosis” and “alcoholic liver cirrhosis” interchangeably refer to liver fibrosis that results from alcohol consuption, and is characterized by excessive accumulation of collagen and other extracellular matrix (ECM) proteins, steatosis, and fibrosis, and is accompanied by release of TGF-β1, mostly produced by BM-derived macrophages and Kupffer cells^{5,6,13-15}. Hepatic stellate cells (HSCs) are the major source of myofibroblasts in hepatotoxic liver injury¹⁶. Under physiological conditions, HSCs store Vitamin A and exhibit a quiescent phenotype (qHSCs), but in response to alcohol-induced injury upregulate α-smooth muscle actin (α-SMA) and collagen Type I¹³. Acetaldehyde, an ethanol metabolite produced by hepatocytes, can directly stimulate HSCs to produce collagen³.

“Hepatocellular carcinoma” (“HCC”) is a malignant tumor made of cells resembling hepatocytes¹⁷, usually arises in a cirrhotic liver^{18,19} of patients with ALD, and is identified by expression of alpha-fetoprotein (AFP), CD90, CD133 and EpCAM^{1,20-22.} Prolonged alcohol consumption has a strong immunosuppressive effect. Accumulating evidence suggests that HCC originates from dedifferentiation and transformation of mature hepatocytes, or from maturation arrest of hepatic progenitors^{19,20}. Progression of HCC is associated with upregulation of IL-6²³, IL-17^{24,25} and IL-22²⁶ and constitutive activation of STAT3²⁷. In addition to STAT3²⁸, NFκB, Wnt/β-catenin, and Hedgehog signaling pathways are implicated in HCC development^{24,29-32}.

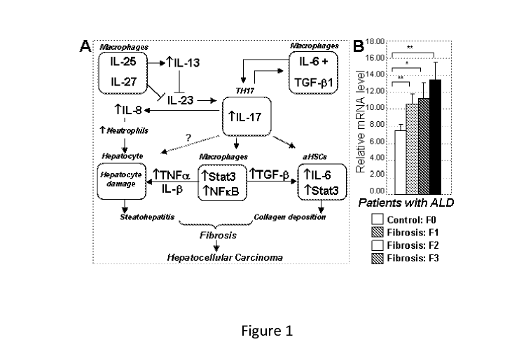

“Interleukin 17” (“IL-17”) includes IL-17A, which is mainly produced by CD4⁺ Th17 cells (FIG. 1A), but also by other cells, including γδ T cells, CD8⁺ T cells, NKT cells, NK cells, innate lymphoid cells, eosinophils, neutrophils, and monocytes³³⁻³⁵. Th17 cells secrete IL-17 cytokines, a family of cytokines comprised of IL-17A, IL-17F, IL-17B, IL-17C and IL-17E³⁶. The IL-17A homodimers (also known as IL-17) are most abundant in Th17 cells, exhibit higher biological activity, and signal through the receptors IL-17RA and IL-17RC³³. IL-17RA is ubiquitously expressed, but is strongly induced in hematopoietic cells³⁷ and fibroblasts³⁸ in response to stress. IL-17A activates inflammatory intracellular signaling in target cells, including TRAF6, Act1, JNK, ERK, NF-κB, STAT3^{37,39}. IL-17 mediates autoimmunity, and the autoimmune inflammatory diseases psoriasis and rheumatoid arthritis respond to anti-IL-17 biological therapies⁴⁰. Most recently, IL-17 has been implicated in liver, lung, and skin fibrosis, and in tumorigenesis^{33,36,41-43,9,44-47}. Although anti-TNF-α therapy has been ineffective in patients with ALD^{1,48}, a corollary of our underlying hypothesis is that anti-IL-17 therapy is a potential novel therapy for ALD (FIG. 1B). “ ”

“Interleukin 22” (“IL-22”) is a protein that in humans is encoded by the IL22 gene. Dumoutier et al. (August 2000). Proc. Natl. Acad. Sci. U.S.A. 97 (18): 10144-9, Xie et al. (October 2000) J. Biol. Chem. 275 (40): 31335-9.

“Interleukin 23” (“IL-23”) is a heterodimer composed of a unique p19 subunit and a p40 subunit⁵⁴. IL-23 is expressed by macrophages and DCs, signals through IL-12Rbeta1 and IL-23R receptors, and activates Jak2/STAT3 signaling pathway⁵⁹. Mice deficient of IL-23p19 have very few Th17 cells^{52,55}, demonstrating that the main biological function of IL-23 is regulation of Th17 cell expansion. We have demonstrated that IL-23 is upregulated along with IL-17 in fibrotic liver, and IL-23^{-/-} deficient mice develop less fibrosis in response to cholestatic and toxic liver injury⁵⁸, indicating that the IL-23/Th17 axis may become a promising target for suppressing liver inflammation during ALD^{60,61}. Furthermore, IL-23 is upregulated in multiple human cancers, and ablation of IL-23p19 gene resulted in reduced tumorigenesis in mouse models of skin cancer⁶² and colitis-associated cancer (CAC)⁶³. IL-23 also promotes HCC development⁶⁴⁻⁶⁶.

“Interleukin 25” (“IL-25” or “IL-17E”). IL-25 propagates allergic responses^{56,57,67}. IL-25 binds to IL-17RA and IL-17RB heterodimers (of which IL-17RB represents an IL-25 specific moiety^{68,69}), and induces Act1-dependent activation of NFκB signaling pathway in target cells⁷⁰. IL-25 drives the expression of IL-13⁷¹, which is required for suppression of Th17 responses^{56,57,67,72,73}. We have demonstrated that IL-25 attenuates liver fibrosis in mice, suggesting that IL-25 agonists may become targets for ALD treatment.

“Interleukin 27” (“IL-27”) is formed from IL-27p28 and Ebi3 subunits⁷⁴⁻⁷⁶. IL-27 signals via IL-27RA and common receptor chain gp130, activating STAT3 and STAT1 in target cells^{74,77}. IL-27 suppresses de novo Th17 cell differentiation driven by IL-6 and TGF-β1⁷⁵.

“Mammalian” subjects include humans, non-human primates, murines, ovines, bovines, ruminants, lagomorphs, porcines, caprines, equines, canines, felines, ayes, etc.). Thus, mammalian subjects are exemplified by mouse, rat, guinea pig, hamster, ferret and chinchilla.

A subject “in need of reducing one or more symptoms of” a disease includes a subject that exhibits and/or is at risk of exhibiting one or more symptoms of the disease. For Example, subjects may be at risk based on family history, genetic factors, environmental factors, etc. This term includes animal models of the disease. Thus, administering a composition (which reduces a disease and/or which reduces one or more symptoms of a disease) to a subject in need of reducing the disease and/or of reducing one or more symptoms of the disease includes prophylactic administration of the composition (i.e., before the disease and/or one or more symptoms of the disease are detectable) and/or therapeutic administration of the composition (i.e., after the disease and/or one or more symptoms of the disease are detectable).

The invention's compositions and methods are also useful for a subject “at risk” for disease refers to a subject that is predisposed to contracting and/or expressing one or more symptoms of the disease. This predisposition may be genetic (e.g., a particular genetic tendency to expressing one or more symptoms of the disease, such as heritable disorders, etc.), or due to other factors (e.g., environmental conditions, voluntary and/or involuntary exposures to detrimental compounds, including alcohol, and carcinogens, present in the environment, etc.). The term subject “at risk” includes subjects “suffering from disease,” i.e., a subject that is currently experiencing one or more symptoms of the disease. It is not intended that the present invention be limited to any particular signs or symptoms. Thus, it is intended that the present invention encompass subjects that are experiencing any range of disease, from sub-clinical symptoms to full-blown disease, wherein the subject exhibits at least one of the indicia (e.g., signs and symptoms) associated with the disease.

The term “administering” a compound to a subject means providing the compound to the using methods known in the art (e.g., Erickson et al., U.S. Pat. No. 6,632,979; Furuta et al., U.S. Pat. No. 6,905,839; Jackobsen et al., U.S. Pat. No. 6,238,878; Simon et al., U.S. Pat. No. 5,851,789). The invention's compositions may be administered prophylactically (i.e., before the observation of disease symptoms) and/or therapeutically (i.e., after the observation of disease symptoms). Administration also may be concomitant with (i.e., at the same time as, or during) manifestation of one or more disease symptoms. Also, the invention's compositions may be administered before, concomitantly with, and/or after administration of another type of drug or therapeutic procedure (e.g., surgery). Methods of administering the invention's compositions include, without limitation, administration in oral, parenteral, intraperitoneal, intranasal, topical and sublingual forms. Parenteral routes of administration include, for example, subcutaneous, intravenous, intramuscular, intrastemal injection, and infusion routes.

The terms “therapeutic amount,” “pharmaceutically effective amount,” “therapeutically effective amount,” “biologically effective amount,” and “protective amount” are used interchangeably herein to refer to an amount that is sufficient to achieve a desired result, whether quantitative and/or qualitative. In particular, a therapeutic amount is that amount that delays, reduces, palliates, ameliorates, stabilizes, prevents and/or reverses one or more symptoms of the disease compared to in the absence of the composition of interest.

Therapeutic “compounds” that may be useful in the invention's methods include, without limitation, the interleukin 17A (IL-17A)-specific human monoclonal antibody secukinumab (AIN457) (described in EP 1776142 B1, published in W02006013107A1) (Novartis, U.S.A.), and has shown promising efficacy for the treatment of psoriasis, rheumatoid arthritis and chronic non-infectious uveitis in Phase II trials. Covagen announced in December 2011 that it is entering preclinical development with its first drug candidate, a first-in-class bispecific TNF/IL-17A inhibitor for the treatment of inflammatory diseases such as rheumatoid arthritis (RA), psoriasis and psoriatic arthritis. This drug candidate comprises an interleukin 17A (IL-17A) neutralizing Fynomer which has been fused to a fully human anti-TNF antibody.

The following compounds may be useful in the methods of the invention, and are currently in clinical trials as IL-17 and IL-17R inhibitors: Secukinumab (AIN457) (described in EP 1776142 B1, published in W02006013107A1) (Novartis, U.S.A.), anti-IL-17 antibody Ixekizumab (LY2439821) (Eli Lilly, USA), Brodalumab (AMG 827), ABT-122, RG4934, SCH-900117, SCH-900222 (MK-3222), Apilimod, Etanercept, and Simvistatin.

The following compounds may be useful in the methods of the invention, and are currently in clinical trials as STAT3 inhibitors: Cetuxima, Panitumumab, Gefitinib, Erlotinib, Lapatinib, AG490, LS-104, ICNB1824, CEP-701, Dasatinib, AZD0530, Bosutinib, Karyostatin 1A, Leptomycin B, Ratjadone A, Guggulsterone, Honokiol, Curcumin, Resveratrol, Flavopiridol, and Cucurbitacin.

The following compounds may be useful in the methods of the invention, and are currently in clinical trials as JAK2 inhibitors: Ruxolitinib (INCB018424), Lestaurtinib (CEP701), Pacritinib (SB1518), SAR302503 (TG101348), CYT-387, LY2784544, XL-019, BMS-91153, NS-018, and AZD1480.

The terms “reduce,” “inhibit,” “diminish,” “suppress,” “decrease,” and grammatical equivalents (including “lower,” “smaller,” etc.) when in reference to the level of any molecule (e.g., interleukin 17 (IL-17, IL 17A), interleukin 23 (IL-23), Stat3, and Jak2, interleukin 22 (IL-22), interleukin 25 (IL-25, IL-17E), interleukin 27 (IL-27), amino acid sequence, and nucleic acid sequence, antibody, etc.), cell, and/or phenomenon (e.g., level of expression of a gene, disease symptom, level of binding of two molecules such as binding of a ligand to its receptor and or to an antibody), specificity of binding of two molecules, affinity of binding of two molecules, disease symptom, specificity to disease, sensitivity to disease, affinity of binding, enzyme activity, etc.) in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than, at least 25% lower than, at least 50% lower than, at least 75% lower than, and/or at least 90% lower than the quantity of the same molecule, cell and/or phenomenon in the second sample (or in the second subject). In another embodiment, the quantity of molecule, cell, and/or phenomenon in the first sample (or in the first subject) is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in the second sample (or in the second subject). In one embodiment, the first sample (or the first subject) is exemplified by, but not limited to, a sample (or subject) that has been manipulated using the invention's compositions and/or methods. In a further embodiment, the second sample (or the second subject) is exemplified by, but not limited to, a sample (or subject) that has not been manipulated using the invention's compositions and/or methods. In an alternative embodiment, the second sample (or the second subject) is exemplified by, but not limited to, a sample (or subject) that has been manipulated, using the invention's compositions and/or methods, at a different dosage and/or for a different duration and/or via a different route of administration compared to the first subject. In one embodiment, the first and second samples (or subjects) may be the same, such as where the effect of different regimens (e.g., of dosages, duration, route of administration, etc.) of the invention's compositions and/or methods is sought to be determined on one sample (or subject). In another embodiment, the first and second samples (or subjects) may be different, such as when comparing the effect of the invention's compositions and/or methods on one sample (subject), for example a patient participating in a clinical trial and another individual in a hospital.

The terms “increase,” “elevate,” “raise,” and grammatical equivalents (including “higher,” “greater,” etc.) when in reference to the level of any molecule (e.g., interleukin 17 (IL-17, IL-17A), interleukin 23 (IL-23), Stat3, and Jak2, interleukin 22 (IL-22), interleukin 25 (IL-25, IL-17E), interleukin 27 (IL-27), amino acid sequence, and nucleic acid sequence, antibody, etc.), cell, and/or phenomenon (e.g., level of expression of a gene, disease symptom, level of binding of two molecules such as binding of a ligand to its receptor and or to an antibody), specificity of binding of two molecules, affinity of binding of two molecules, disease symptom, specificity to disease, sensitivity to disease, affinity of binding, enzyme activity, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject). In one embodiment, the first sample (or the first subject) is exemplified by, but not limited to, a sample (or subject) that has been manipulated using the invention's compositions and/or methods. In a further embodiment, the second sample (or the second subject) is exemplified by, but not limited to, a sample (or subject) that has not been manipulated using the invention's compositions and/or methods. In an alternative embodiment, the second sample (or the second subject) is exemplified by, but not limited to, a sample (or subject) that has been manipulated, using the invention's compositions and/or methods, at a different dosage and/or for a different duration and/or via a different route of administration compared to the first subject. In one embodiment, the first and second samples (or subjects) may be the same, such as where the effect of different regimens (e.g., of dosages, duration, route of administration, etc.) of the invention's compositions and/or methods is sought to be determined on one sample (or subject). In another embodiment, the first and second samples (or subjects) may be different, such as when comparing the effect of the invention's compositions and/or methods on one sample (subject), for example a patient participating in a clinical trial and another individual in a hospital.

“Antibody” refers to an immunoglobulin (e.g., IgG, IgM, IgA, IgE, IgD, etc.) and/or portion thereof that contains a “variable domain” (also referred to as the “F_{V} region”) for binding to antigens. In one embodiment, the invention's antibodies are monoclonal antibodies produced by hybridoma cells. In particular, the invention contemplates antibody fragments that contain the idiotype (“antigen-binding fragment”) of the antibody molecule. For example, such fragments include, but are not limited to, the Fab region, F(ab′)2 fragment, pFc′ fragment, and Fab′ fragments.

“Monoclonal antibody” (“MAb”) refers to an antibody produced from a single clone of plasma cells.

“Humanized” forms of non-human (e.g., murine) antibodies are antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody may also comprise at least a portion of an immunoglobulin constant region (Fc), typically that of a human immunoglobulin. Examples of methods used to generate humanized antibodies are described in U.S. Pat. No. 5,225,539 to Winter et al. (herein incorporated by reference).

Importantly, early methods for humanizing antibodies often resulted in antibodies with lower affinity than the non-human antibody starting material. More recent approaches to humanizing antibodies address this problem by making changes to the CDRs. See U.S. Patent Application Publication No. 20040162413, hereby incorporated by reference. In some embodiments, the present invention provides an optimized heteromeric variable region (e.g. that may or may not be part of a full antibody other molecule) having equal or higher antigen binding affinity than a donor heteromeric variable region, wherein the donor heteromeric variable region comprises three light chain donor CDRs, and wherein the optimized heteromeric variable region comprises: a) a light chain altered variable region comprising; i) four unvaried human germline light chain framework regions, and ii) three light chain altered variable region CDRs, wherein at least one of the three light chain altered variable region CDRs is a light chain donor CDR variant, and wherein the light chain donor CDR variant comprises a different amino acid at only one, two, three or four positions compared to one of the three light chain donor CDRs (e.g. the at least one light chain donor CDR variant is identical to one of the light chain donor CDRs except for one, two, three or four amino acid differences).

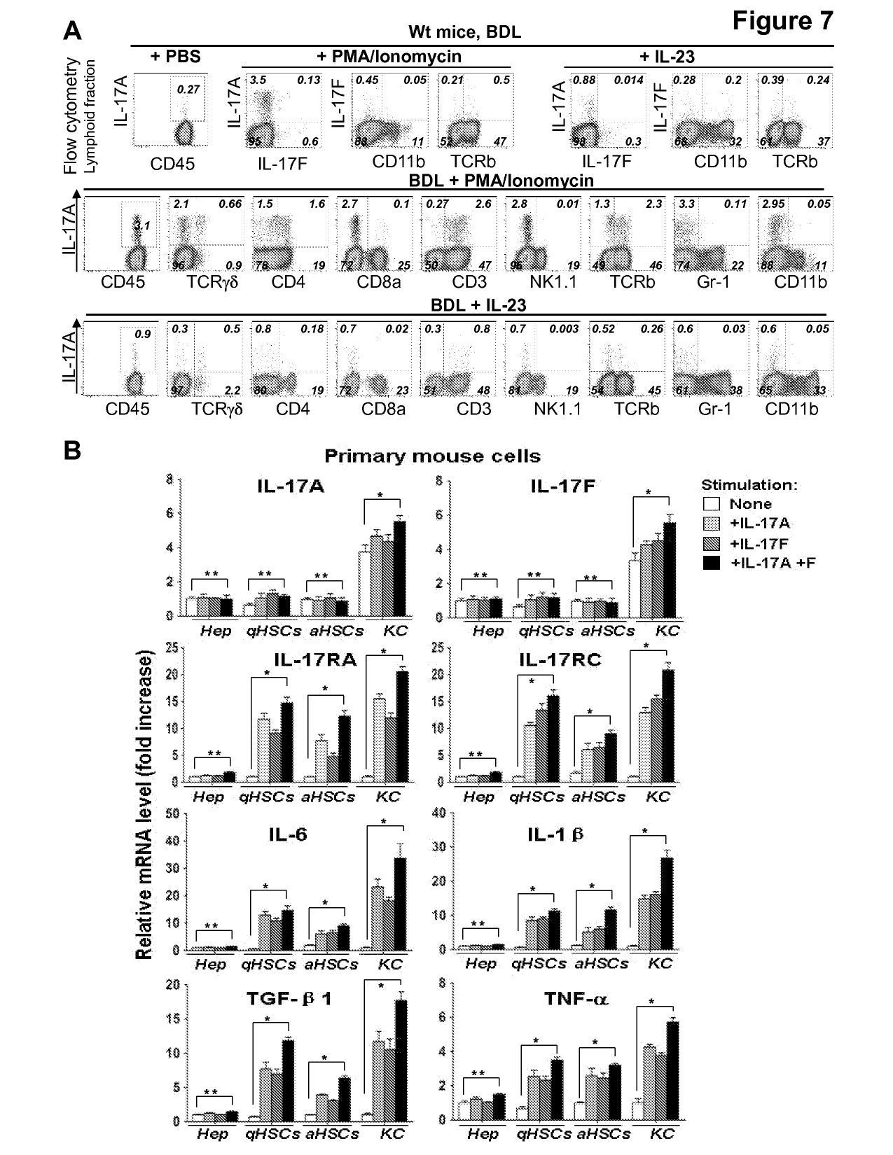

“Immune cell” refers to a white blood cell that includes B-cell that is involved in antibody-mediated immunity and/or T-cell that is involved in cell-mediated immunity, and/or natural killer (NK) cell that is involved in the innate immune system in defending the host from both tumors and virally infected cells. Immune cell is exemplified by CD45⁺, CD3⁺, CD4⁺, CD8a⁺, TCRγδ⁺, TCRβ⁺, NK1.1⁺, CD11b⁺ cells (FIG. 7).

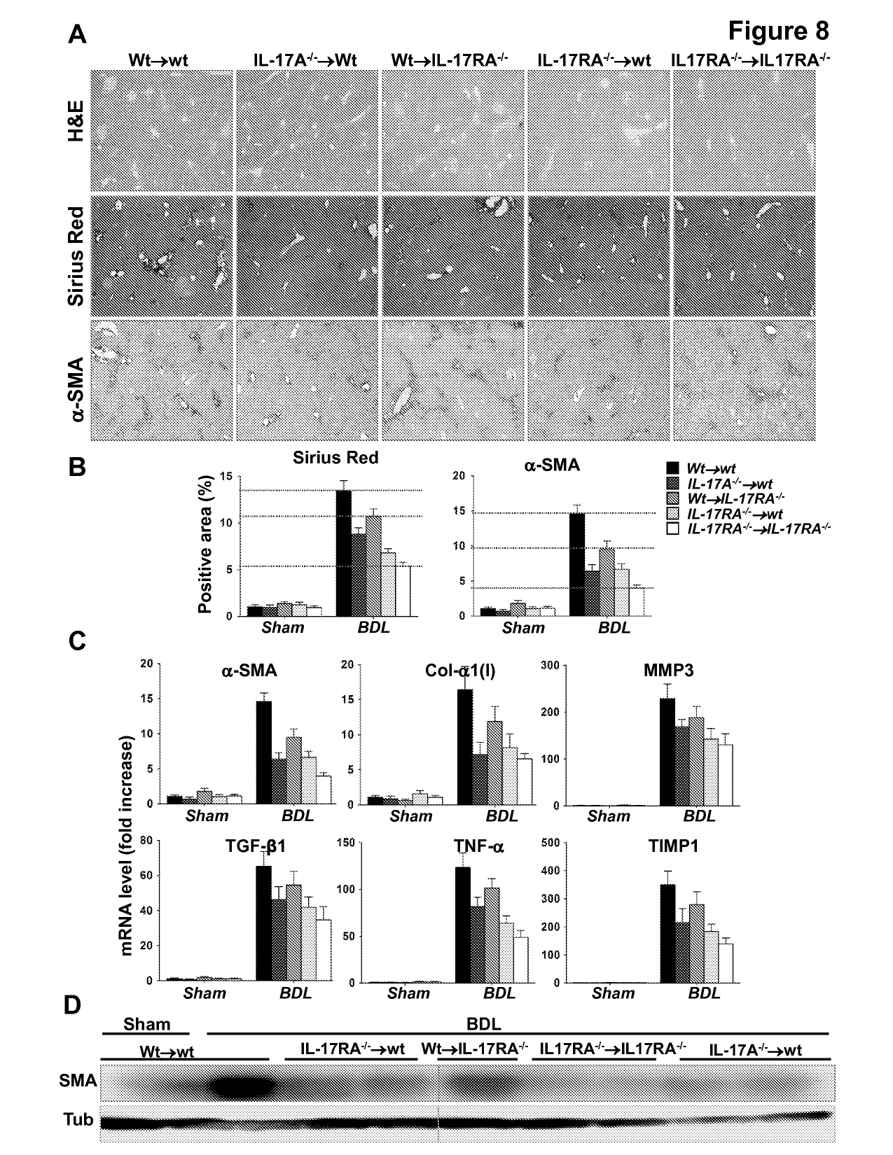

“Inflammatory cell” refers to a neutrophil, macrophage, monocyte, eosinophil and/or basophil cell participating in the inflammatory response to a foreign substance (FIG. 8).

“Hepatic stellate cells” and “HSCs” interchangeably refer to pericytes found in the perisinusoidal space (a small area between the sinusoids and hepatocytes) of the liver also known as the space of Disse. The stellate cell is the major cell type involved in liver fibrosis. Data herein show that HSCs responded to IL-17 by increasing the mRNAs of IL-17RA and IL-17RC, and expression of fibrogenic cytokines TGF-β1, IL-6, and TNF-α (FIG. 7B, FIG. 15, and FIG. 16).

“Kupffer cells,” “KCs” and “stellate macrophages” are specialized macrophages located in the liver lining the walls of the sinusoids that form part of the reticuloendothelial system (RES) (or mononuclear phagocyte system). Data herein demonstrate that KCs responded to IL-17 by increasing the mRNAs of IL-17RA and IL-17RC, and expression of fibrogenic cytokines TGF-β1, IL-6, and TNF-α (FIG. 7B and FIG. 16).

### DESCRIPTION OF THE INVENTION

The invention provides methods and compositions for reducing symptoms of steatohepatitis and/or liver fibrosis and/or hepatocellular carcinoma (HCC) in a mammalian subject in need thereof, comprising administering to the mammalian subject a therapeutic amount of a compound that reduces the level of interleukin 17 (IL-17) and/or interleukin 23 (IL-23) and/or signal transducer and activator of transcription 3 (Stat3) and/or Janus kinase 2 (Jak2). The invention's methods may comprise administering to the mammalian subject a therapeutic amount of a compound that increase the level of interleukin 22 (IL-22) and/or interleukin 25 (IL-25) and/or interleukin 27 (IL-27). The invention's methods may comprise administering to the mammalian subject a therapeutic amount of interleukin 22 (IL-22) and/or interleukin 25 (IL-25) and/or interleukin 27 (IL-27).

Currently, there is no optimal treatment for patients with liver fibrosis. The only available treatment for patients with ALD-induced liver fibrosis is alcohol abstinence and liver transplantation, and targeted-therapies are urgently needed. The development of such therapies is hampered by poor knowledge of the underlying cellular and molecular mechanisms of ALD. The below Examples identify new targets to prevent and revert progression of ALD from steatohepatitis to fibrosis and HCC.

In particular, the Examples below characterized the role of IL-17 signaling and related cytokines in hepatic fibrosis induced by either bile duct ligation (BDL) or carbon tetrachloride (CCl₄) (Bataller & Brenner 2005). Data herein demonstrate that IL-17RA deletion in mice dramatically inhibits both models of liver fibrosis. Data herein (e.g., Examples 2-14) demonstrate that using BM chimeric mice, deletion of IL-17A, IL-23 or IL-17RA in BM-derived cells decreases liver fibrosis by 50-55%, while deletion of IL-22 exacerbates fibrosis. Also, data herein (e.g., Examples 2-14) demonstrate that IL-17A directly stimulates activation of HSCs in a Stat3-dependent manner.

The role of IL-17 in liver fibrosis, Kupffer cells, and Hepatic Stellate cells has not been evaluated previously. Here we examined the role of IL-17 signaling in the pathogenesis of liver fibrosis.

Using models of cholestatic and toxic liver injury, the development of liver fibrosis in wild type mice was compared to IL-17RA^{-/-} and bone marrow chimeric mice (Examples 2-14). We determined that IL-17 has a strong pro-fibrogenic effect, and ablation of IL-17RA signaling in mice results in inhibition of liver fibrosis by 75% compared to the wild type mice. We characterized the mechanism underlying this process and demonstrate that IL-17A regulates production of TGF-β1 by Kupffer cells, which in turn, induce activation of HSC into fibrogenic myofibroblasts via Smad2/3 signaling pathway, and further facilitate differentiation of IL-17A-producing cells. In addition, IL-17A directly activates HSCs via the Stat3 signaling pathway (Examples 2-14). Thus, IL-17A induces liver fibrosis through multiple mechanisms and may serve as an attractive target for anti-fibrotic therapy.

There are very few cytokines described to date which exhibit strong fibrogenic effects. In addition to TGF-β1, PDGF, EGF, HGF, IL-13, and leptin, IL-17 demonstrates compelling pro-fibrogenic features, which strongly affects development of liver fibrosis. Here we demonstrate for the first time that the IL-17-Stat3 axis may be important for fibrogenic activation of HSCs. Consistent with this, deletion of Stat3 in HSCs attenuates development of liver fibrosis in GFAP^{Stat3-/-} mice. We are currently investigating the role of Jak2, the Stat3 phosphorylating kinase, in activation of HSCs in the course of liver fibrosis in GFAP^{Jak2-/-} mice. Data herein will broaden our understanding of the pathophysiology of liver fibrosis, and add IL-17A-Stat3 signaling pathway to the list of potential therapeutic targets.

Our data indicates that IL-17-Stat3-Jak2 pathway is a target for anti-fibrotic therapy.

Data herein demonstrate that ablation of IL-17 signaling in IL-17RA-deficient mice inhibits development of liver fibrosis by 75%. This effect is achieved by inhibition of IL-17 signaling pathway in immune cells and liver resident fibrogenic myofibroblasts (Hepatic stellate cells). Data herein demonstrate that inhibition of IL-17 signaling in immune and inflammatory cells reduces fibrosis in mice by 50%. Inhibition of IL-17 signaling in Hepatic stellate cells reduces fibrosis in mice by 25%. Thus, data herein demonstrate that IL-17 is a target for anti-fibrotic therapy.

In addition to TGF-beta, very few cytokines have been demonstrated to activate collagen production in Hepatic Stellate cells. Here we demonstrate that inhibition of IL-17-Stat3-Jak2 signaling pathway in Hepatic Stellate cells is pro-fibrogenic and can serve as an attractive therapeutic target to attenuate activation of fibrogenic myofibroblasts.

We have demonstrated IL-17 signaling in liver resident macrophages (Kupffer cells) regulates 30% of total TGF-beta production during development of liver fibrosis Inhibition of IL-17 in Kupffer cells will decrease development of liver fibrosis.

IL-17 signaling has been implicated in lung and skin fibrosis. Here we examined the role of IL-17 signaling in the pathogenesis of liver fibrosis. Using cholestatic and hepatotoxic models of liver injury, the development of liver fibrosis in wild type mice was compared to IL-17RA^{-/-} mice, and to bone marrow chimeric mice devoid of IL-17 signaling in immune cells and Kupffer cells (IL-17RA^{-/-}→wt and IL-17A^{-/-}→wt mice generated by transplanation of IL-17RA−/− (or IL−17A−/−) bone marrow into lethaly irradiated wild type mice), or in liver resident cells (Wt→IL-17RA^{-/-} mice generated by transplantation of the wild type BM into IL-17RA−/− mice). We determined that IL-17 and its receptor is highly induced in liver injury and has a strong pro-fibrogenic effect on both inflammatory and liver resident cells. IL-17 signaling facilitates production of IL-6, IL-β, and TNF-α by inflammatory cells, and increases the expression of TGF-β1, the major pro-fibrogenic cytokine. Furthermore, IL-17 directly induces hepatic stellate cells to produce collagen Type I and promotes their activation into myofibroblasts via the Stat3 signaling pathway. Furthermore, mice devoid of Stat3 signaling in HSCs, GFAP^{Stat3-/-} mice, are less susceptible to fibrosis. Thus, IL-17 induces liver fibrosis through multiple mechanisms and may serve as an attractive target for anti-fibrotic therapy.

There are many inhibitors already developed and used in clinical practice, which target IL-17, Stat3 and Jak2. In addition to autoimmune diseases, and myelofibrosis, these inhibitors can be potentially used for anti-fibrotic therapy.

To summarize some aspects of the invention:

1. IL-17-Stat3-Jak2 pathway is a target for anti-fibrotic therapy: Using three models of liver fibrosis (toxic liver injury induced by carbon tetrachloride, and alcohol induced liver injury, and cholestatic liver injury) we have demonstrated that IL-17 plays an important role in pathogenesis of liver fibrosis. In addition, alcoholic liver injury in patients induces alcoholic steatohepatitis, alcoholic fibrosis and hepatocellular carcinoma (HCC). We determine, that upregulation of IL-17 is associated with progression of each stage (Examples 2-17). Futhermore, IL-17RA−/− mice are protected from progression of alcoholic liver disease from steatohepatitis to fibrosis(Example 16). Therefore, blocking IL-17 will not only prevent development of alcohol-induced liver fibrosis, but will also prevent development of HCC.

2. IL-17-Stat3-Jak2 pathway is a target for anti-fibrotic therapy: we demonstrate that IL-17 directly induces activation and collagen Type I production in Hepatic Stellate cells via Stat3-Jak2 signaling pathway. Consistent with this, deletion of Stat3 in Hepatic Stellate cells results in attenuation of liver fibrosis in mice. Several other cytokines also utilize Stat3-Jak2 signaling pathway. Thus, inhibition of IL-23 in mice attenuates liver fibrosis of different etiologies, while administration of IL-25 in mice attenuates liver fibrosis via inhibition of IL-23-dependent IL-17 production.

3. Our data demonstrate that progression of liver fibrosis to liver cancer can be controlled by IL-17, IL-23, IL-25, IL-22 cytokines Inhibition of IL-23 in mice attenuates liver fibrosis of different etiologies, while administration of IL-25 in mice attenuates liver fibrosis via inhibition of IL-23-dependent IL-17 production. Although, IL-22 is not directly involved in IL-17 pathway, administration of IL-22 inhibits liver fibrosis, since IL-22 facilitate survival of damaged hepatocytes. Data herein demonstrate that blocking of IL-17 and transient increase of IL-22 production provides an important target for anti-fibrotic therapy. Furthermore, complete abrogation of IL-17 production may cause unwanted complications (inability to combat infection). Therefore, an alternative pathway to suppress IL-17 production is suggested. In one embodiment, production of IL-23 is blocked, and/or production of IL-25 is facilitated. Induction of IL-25 production will result in suppression of IL-23, and prevention of expansion of IL-17-producing cells. Alternatively to IL-25, IL-27 can be used to suppress IL-23 production.

4. IL-22 improved survival of damaged hepatocytes, and therefore can be used to reduce liver damage. In combination with IL-17 suppression, IL-22 dramatically attenuates liver fibrosis.

5. IL-25 and IL-27 suppress IL-23-dependent expansion of IL-17-producing T cells.

Altered levels of molecules (such as reduced levels of one or more of (a) interleukin 17 (IL-17), (b) interleukin 23 (IL-23), (c) signal transducer and activator of transcription 3 (Stat3), and (d) Janus kinase 2 (Jak2), and/or increased levels of one or more of (a) interleukin 22 (IL-22), (b) interleukin 25 (IL-25), and (c) interleukin 27 (IL-27)) may be accomplished using RNAi, siRNA, microRNA, antibodies, immunologic fragments and constructs, aptamers, pseudosubstrates, peptidomimetics and competitive inhibitor analogs.

A. Alcoholic Liver Disease (ALD) Therapy:

The only available treatment for patients with ALD-induced liver fibrosis is alcohol abstinence and liver transplantation, and targeted-therapies are urgently needed. The development of such therapies is hampered by poor knowledge of the underlying cellular and molecular mechanisms of ALD. The experiments described herein are aimed to identify new targets to prevent and revert progression of ALD from steatohepatitis to fibrosis and HCC.

1) the Effect of IL-17 Inhibition on ALD.

The role of IL-17 in the pathogenesis of ALD will be characterized and the therapeutic potential of targeting IL-17 will be explored. In one embodiment, blocking IL-17 or cytokines that promote Th17 induction could be used for treatment of ALD.

2) Alternative Pathways to Inhibit IL-17 in ALD.

Prolonged suppression of Th17 may cause increased susceptibility to infections⁴³. Therefore, alternative pathways are needed to inhibit expansion of Th17 and prevent increased secretion of IL-17. We will determine if blocking IL-23 production can effectively prevent Th17 expansion. We will also test if IL-25 and IL-27 agonists can block IL-23 production and in this way prevent Th17 expansion.

3) Application of IL-17 Inhibitors for ALD Treatment.

There is a growing body of evidence that the IL-17 signaling pathway contributes to the pathogenesis of liver diseases of many etiologies^{24,47,58,83,84.} Remarkably, many novel inhibitors of IL-17 have been developed and are successfully used to treat psoriasis.⁴⁰ Anti-IL-17 (ixekizumab, Eli Lilly) and IL-23p19 (Merck) antibodies are currently successfully used in clinical trials in patients with psoriasis⁸⁵. Similar approaches could be beneficial as therapy for patients with ALD.

B. Models to Study Alcoholic Liver Disease (ALD).

Because of the clinical importance of ALD, we have employed three models of ALD in mice that mimic ALD stages in patients, and allow the monitoring of progression from steatohepatitis to fibrosis and to HCC. These models effectively reflect pathological changes in patients with alcoholic liver disease.

ALD studies have been hampered by the absence of suitable animal models. First, there are differences in time courses (months in mice versus years in human patients). Second, most models of chronic alcohol feeding in mice (such as Lieber-Decarli diet) do not mirror the stages of ALD in patients. The Examples described herein study the pathogenesis of ALD and factors (including IL-17 signaling) that mediate ALD progression using a collective model of ALD progression from steatosis to steatohepatitis, to fibrosis, and to hepatocellular carcinoma. Recently, we developed a chronic-binge ethanol feeding model, which induces significant liver inflammation and neutrophil infiltration^{12,78} (but no fibrosis), and successfully analyzed the ALD stages of steatosis and steatohepatitis. Serum levels of about 250IU/L ALT and 420 IU/L AST were found post binge gavage, and correlate with increased expression of hepatic and serum inflammatory cytokines and hepatic oxidative stress.

C. Assessment of IL-17 in Alcoholic Liver Disease (ALD).

Recent studies have demonstrated that IL-17 and Th17-regulating cytokines (IL-23, IL-25 and IL-27), may play an important role in the pathogenesis of liver fibrosis and cancer. Using knockout mice we will dissect the effect of IL-17 and the cytokines that are critical for Th17 expansion (IL-23, IL-25, IL-27, IL-6) on steatohepatitis, fibrosis and HCC.

Using genetic mouse models, we will further determine if inhibition of IL-17 signaling has therapeutic effect on progression of steatosis to steatohepatitis, fibrosis, and HCC. While not intending to limit the invention to any particular mechanism, our central hypothesis is that IL-17 promotes development of ALD from steatohepatitis to fibrosis and HCC. The experiments described herein were designed to determine if blocking IL-17 production can prevent ALD progression.

A major strength of the experiments described herein is that three mouse models of ALD are used to study the progression of alcoholic steatohepatitis to fibrosis and HCC similar to that observed in patients. Our strategy is to use several knockout mice, including cell-specific knockout mice to 1) determine the overall role of IL-17 in ALD; 2) to dissect the specific mechanism(s) of IL-17 action at each stage of ALD; 3) evaluate the pathways of IL-17 regulation, and identify potential effect(s) of Th17-regulating cytokines on IL-17 production; and 4) determine the role of each Th17-regulating cytokine on individual stages of ALD.

### EXPERIMENTAL

The following examples serve to illustrate certain preferred embodiments and aspects of the present invention and are not to be construed as limiting the scope thereof.

### Example 1

### Materials and Methods Used in Examples 2-14

Cell Lines and Mice:

LX-2 cell line¹² and hTERT cell line¹³, Collagen α1(I)-GFP mice¹⁴ were previously described. C57BL/6 mice (8 weeks old) and GFAP-Cre mice were purchased (Jackson Laboratories). We obtained IL-17RA^{-/-} mice¹⁵, IL-17A^{-/-} mice¹⁶, and STAT3^{f/f} mice¹⁷, IL-22^{-/-} mice and IL-23^{-/-} mice (Genentech). All animal experiments were approved by the UCSD Institutional Animal Care and Use Committee.

Liver Injury:

Liver injury was induced in mice by intragastric gavage with CCl₄ (1:4 dilution in corn oil, 200 μl×12 injections²) or by BDL (3 weeks)².

Isolation of Hepatocytes and Non-Parenchymal Cell Fraction and Primary HSCs:

Livers are perfused using pronase/collagenase method. Singe-cell suspensions are centrifuged at 50g for 5 minutes to pellet the hepatocyte fraction. The remaining non-parenchymal cell fraction was collected. KC and EC were isolated by gradient centrifugation (15% Nycodenz) following by magnetic sorting with anti-CD11b and anti-CD31 antibodies, respectively (Miltenyi Biotec). HSCs were isolated using gradient centrifugation (8.2% Nycodenz, see Suppl. Methods). RT-PCR and real time quantitative PCR. Quiescent HSCs (5×10⁵ cells, cultured <6 h); activated HSCs, KC and hepatocytes (cultured for 48 h) were serum starved (for 24 h) and then stimulated with mIL-17A (10 ng/ml) and mIL-17F (10 ng/ml) for 2 h. LX2 and hTERT cells (5×10⁵ cells) were stimulated with hIL-17A (10 ng/ml) and hIL-17F (10 ng/ml) for 8h. mRNA was isolated and RT-PCR was performed as described in Suppl. Methods.

Immunohistochemistry and Immunofluorescent Staining.

Human tissues are previously described.¹⁸ Immunohystochemistry was performed on formalin-fixed livers with anti-α-SMA Ab (Dako Cytomation) using MOM kit (Vector Laboratories) following by DAB staining (Vector), and counterstaining with Haematoxilin. Anti-mouse Stat3 (Cell Signaling Technologies, Inc.), Stat3, NF-κB (p65), and phospho-Stmad2/3 (Santa Cruz) and anti-human IL-17A, Stat3 (Santa Cruz) antibodies were used. The images were taken using confocal microscopy and analyzed by Image J.

Bone Marrow Transplantation.

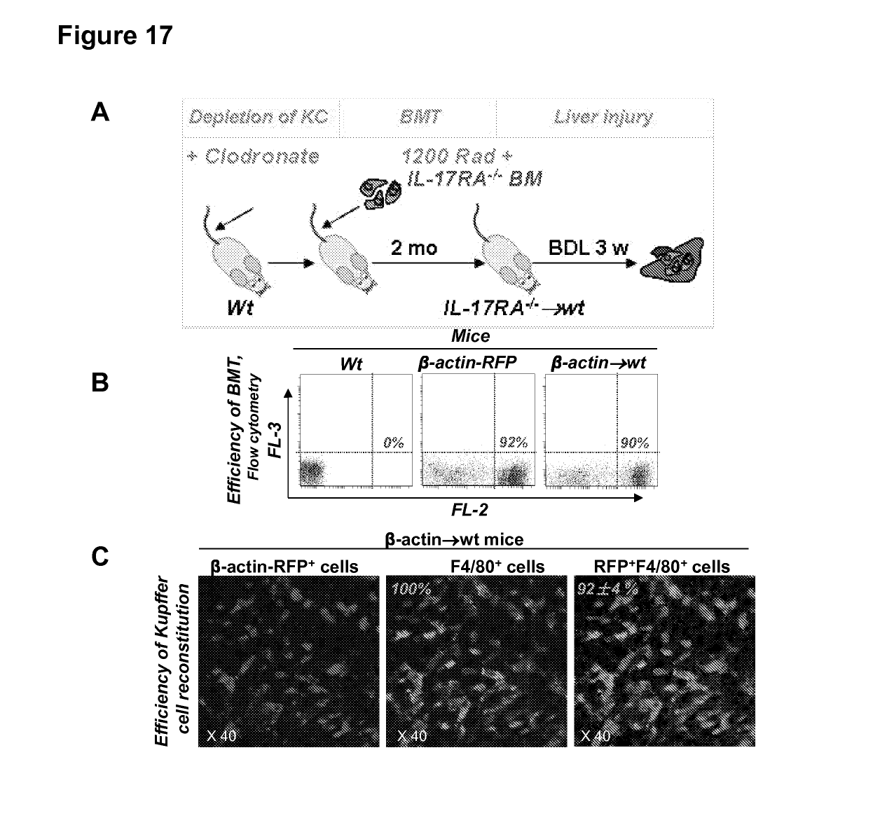

Recipient mice were intravenously injected with liposomal clodronate (150 μl) to deplete KC². BMT was performed 24 h later as described¹⁹, by transplantation of the whole BM into lethally irradiated (1200 Rad) recipient mice. The efficiency of reconstitution was accessed in mice transplanted with β-actin-GFP BM (See FIG. 17).

Flow Cytometry:

Phenotyping was performed on BD LSR II (BD) using anti-mouse antibodies (eBioscience; see Suppl. Materials). Intracellular staining was performed after restimulation of cells with PMA/Ionomycin/Brefeldin A (or IL-23/Brefeldin A) for 5 hours using BD Cytofix/Cytoperm (BD, San Jose, Calif.).

Protein Detection.

Western blot analysis was performed using protein lysates from IL-17A-stimulated primary cells or cell lines (see Suppl. Methods). Serum expression of IL-17A, IL-17F, IL-17E (IL-25), IL-22, and IL-6 was detected by ELISA (IL-17A, IL-17E(IL25), IL-22, and IL-6 antibodies from Biolegend; IL-17F antibodies from eBioscience).

Statistical Analysis.

All data are expressed as the mean±standard deviation (SD). Differences between experimental and control groups were assessed by two-tailed unpaired Student's t-tests using Graph Pad Prism 5.0 (GraphPad Software, San Diego, Calif.). A p-values less than 0.05 was considered statistically significant.

Reagents:

IL-17A, IL-17F, IL-22, IL-23, TGF-β1, IL-6, Leptin were purchased from R&D. Anti-IL-6 Ab was purchased from eBioscience.

Detection of IL-17RA mRNA in Patients with Alcohol-Induced Liver Fibrosis.

Liver biopsy specimens were obtained during routine diagnostic and immediately frozen in liquid nitrogen. Alcoholic liver lesions were assessed according to Kleiner D E, Brunt E M, Van Natta M, et al. Design and validation of a histological scoring system for nonalcoholic fatty liver disease. Hepatology 2005; 41:1313-21., and fibrosis stages were defined as F0 (n=2), F1 (n=5), F2 (n=5), F3 (n=4). For quantification of mRNA expression, total RNA was isolated from snap frozen tissues mRNA was using the RNeasy kit (Qiagen, Basel, Switzerland) and converted to cDNA. Quantitative real-time PCR was performed on the ABI 7700 Sequence Detector (Applied Biosystems, Rotkreuz, Switzerland) with ready-to-use primers and probe kits for human IL-17RA (Applied Biosystems, Rotkreuz, Switzerland), and normalized to GAPDH. The human experiments were approved by the local Ethics Committee of the University of Heidelberg, Germany, where all biopsies were carried out; all patients gave written informed consent to have their tissues included in the experiments.

Primary Cell Cultures:

Primary HSCs and KC were isolated from the wild type and were used for flow cytometry analysis or cultured on uncoated plastic tissue culture dishes in Dulbecco's modified Eagle's medium (DMEM; Gibco-BRL, Grand Island, N.Y.) supplemented with 10% fetal calf serum (FCS), 2 mM L-glutamine and standard antibiotics. Hepatocytes were cultured on collagen-coated plastic plates in Waymouth's medium supplemented with 10% FCS.

RT-PCR and Real Time Quantitative PCR.

mRNA was isolated and RT-PCR was performed using ABI 7000 sequence detection system (Applied Biosystems), specific primers and SYBRGreen. Ct values of each sample were normalized to 18s mRNA expression. Values were expressed as fold induction in comparison with untreated or sham controls. Primers used for RT-PCR:

| Gene | RefSeq ID: | Forward | Reverse |
|---|---|---|---|
| m IL- | NM_008359 | CTTGACTCTGCAG | ATGGCTGCTTCTG |
| l7Ra | | CTCAGCC | CTGCT |
| m IL- | NM_134159 | CCTGCTCCTCAGA | ATCTGGTCCTACA |
| l7Rc | | GACATCC | CGAAGCC |
| m IL- | NM_010552 | TGAGCTTCCCAGA | TCCAGAAGGCCCT |
| 17A | | TCACAGA | CAGACTA |
| m IL- | NM_145856 | TTGATGCAGCCTG | AATTCCAGAACCG |
| 17F | | AGTGTCT | CTCCAGT |
| m IL-6 | NM_031168 | ACCAGAGGAAATT | TGATGCACTTGCA |
| | | TTCAATAGGC | GAAAACA |
| m IL-1-β | NM_008361 | GGTCAAAGGTTTG | TGTGAAATGCCAC |
| | | GAAGCAG | CTTTTGA |
| m TGF- | NM_011577 | CAACCCAGGTCCT | GGAGAGCCCTGGA |
| β1 | | TCCTAAA | TACCAAC |
| m TNF- | NM_013693 | AGGGTCTGGGCCA | CCACCACGCTCTT |
| α | | TAGAACT | CTGTCTAC |
| m | NM_010809 | AGCCTTGGCTGAG | CGATGATGAACGA |
| MMP3 | | TGGTAGA | TGGACAG |
| m | NM_007742 | TAGGCCATTGTGT | ACATGTTCAGCTT |
| COLIA1 | | ATGCAGC | TGTGGACC |
| m | NM_001044384 | AGGTGGTCTCGTT | GTAAGGCCTGTAG |
| TIMP1 | | GATTCGT | CTGTGCC |
| m α- | NM_007392 | GTTCAGTGGTGCC | ACTGGGACGACAT |
| SMA | | TCTGTCA | GGAAAAG |
| m18S | NM_025447 | AGTCCCTGCCCTT | CGATCCGAGGGCC |
| rRNA | | TGTACACA | TCACTA |
| h IL-6 | NM_000600 | GTCAGGGGTGGTT | AGTGAGGAACAAG |
| | | ATTGCAT | CCAGAGC |
| h IL-8 | NM_000584.2 | AAATTTGGGGTGG | TCCTGATTTCTGC |
| | | AAAGGTT | AGCTCTGT |
| h TGF- | NM_000660.3 | GCCCTGGACACCA | AGGCTCCAAATGT |
| β1 | | ACTATTGCT | AGGGGCAGG |
| h | NM_000088.3 | CACACGTCTCGGT | AAGAGGAAGGCCA |
| COL1A1 | | CATGGTA | AGTCGAG |
| mIL-22 | — | ACAGGTTCCAGCC | GTCGTCACCGCTG |
| | | CTACATG | ATGTG |
| mIL-25 | — | CCAGGTGGTTGCA | TGGCTGTAGGTGT |
| | | TTCTTGG | GGGTTCC |
| mIL-23 a | — | GAACGCACATGCA | TGCAAGCAGAACT |
| | | CCAGCGG | GGCTGTTGTCC |
| h TIMP1 | NM_003254.2 | AGGTGGTCTCGTT | GTAAGGCCTGTAG |
| | | GATTCGT | CTGTGCC |
| h SMA | NM_001141945.1 | CCAGAGCCATTGT | CAGCCAAGCACTG |
| | | CACACAC | TCAGG |
| h IL- | NM_002190.2 | CACTTTGCCTCCC | ACCAATCCCAAAA |
| 17A | | AGATCAC | GGTCCTC |
| h IL-17F | NM_052872.3 | CCTCACTCAGAAA | AGCGCAACATGAC |
| | | GGCAAGC | AGTGAAG |

Whole Mouse Genome Gene Expression Microarray:

The gene expression profile of HSCs was studied using Whole Mouse Genome Microarray (Agilent). For this purpose, Vitamin A ‘YFP’ and Vitamin A ⁺YFP⁻ HSCs were sort purified from Collagen-α2(I)^{Cre-YFP} mice with no injury, after CCl₄ (2 mo), and after 7 days or 1 mo recovery from CCl₄. mRNA was purified using RNAeasy columns (Qiagen, Valencia, Calif.), 160 ng of purified RNA per sample was labeled using the LRILAK PLUS, two color low RNA input Linear Amplification kit and hybridized to a Whole Mouse Genome Microarray 4×44K 60 mer slide according to the manufacturer's instructions (Agilent, Santa Clara, Calif.). Slides were scanned using the Agilent GZ505B Scanner and analyzed using the Gene Spring Software (Agilent). Hierarchical clustering of gene expression values was performed using Cluster 3.0 (de Hoon M J, Imoto S, Nolan J, et al. Open source clustering software. Bioinformatics 2004; 20:1453-4) using the correlation coefficient as the similarity metric, and average linkage when merging nodes during tree building. Clustering was performed on genes expressed in at least one condition (>9 log₂ intensity value) to remove absent genes and genes exhibiting a standard deviation greater than 0.75 among log₂ intensity values to remove genes with constant expression. Hierarchical clustering results were visualized using Java Tree View (Saldanha A J. Java Treeview—extensible visualization of microarray data. Bioinformatics 2004; 20:3246-8). Differentially regulated genes were defined as those with significant absolute expression (>9 log₂ intensity value) and exhibiting 2-fold compared to the maximal value in all other samples. Gene ontology and KEGG pathway functional enrichment analysis was performed using DAVID (Dennis G, Jr., Sherman B T, Hosack D A, et al. DAVID: Database for Annotation, Visualization, and Integrated Discovery. Genome Biol 2003; 4:P3).

Western blot analysis was performed using protein lysates from IL-17A-stimulated primary cells or cell lines. Phospho-ERK, total ERK, phospho-AKT (on Ser473), AKT1, phospho-JNK, total-JNK, phospho-NF-κB (p65), NF-κB (p65), phospho-Stat3, Stat3 (Cell Signaling Technologies, Inc.) and α-Tubulin (Santa Cruz) were used and visualized them by the enhanced chemiluminescence light method (Amersham Biosciences).

Flow Cytometry:

Lymphoid fraction from untreated (n=3) and BDL-injured (n=3) wild type mice were in vitro stimulated with PMA (100 ng/ml)+Ionomycin (500 ng/ml)+Brefeldin A (or IL-23+Brefeldin A) for 5h, and analyzed by flow cytometry for intracellular IL-17A and IL-17F expression. Phenotyping of the lymphoid cell fraction isolated from mouse liver was performed on BD LSR II (BD Bioscience Flow Cytometry Systems, BD) using anti-mouse CD90.2-PE, CD4PE, CD3-PE, CD8α-FITC, CD8α-APC, CD4-PerCp-Cy5.5, CD11b-eFluor™ 450, CD11b-FITC, B200-APC, CD11b-PE-Cy5, NK1.1-PE-Cy7, CD45R(B220)-PE-Cy7, F4/80-PerCP-Cy5.5, F4/80-PE, CD11c-PE-Cy7, CD11c-APC, CD11c-PE, CD8a-FITC, IL-17A-Alexa647, IL-17F-PE Abs (eBioscience, San Diego, Calif.). Intracellular staining was performed using BD Cytofix/Cytoperm fixation and Permeabilization Solution (BD, San Jose, Calif.).

### Example 2

### Progression of Liver Fibrosis Correlates with Elevated Expression of IL-17

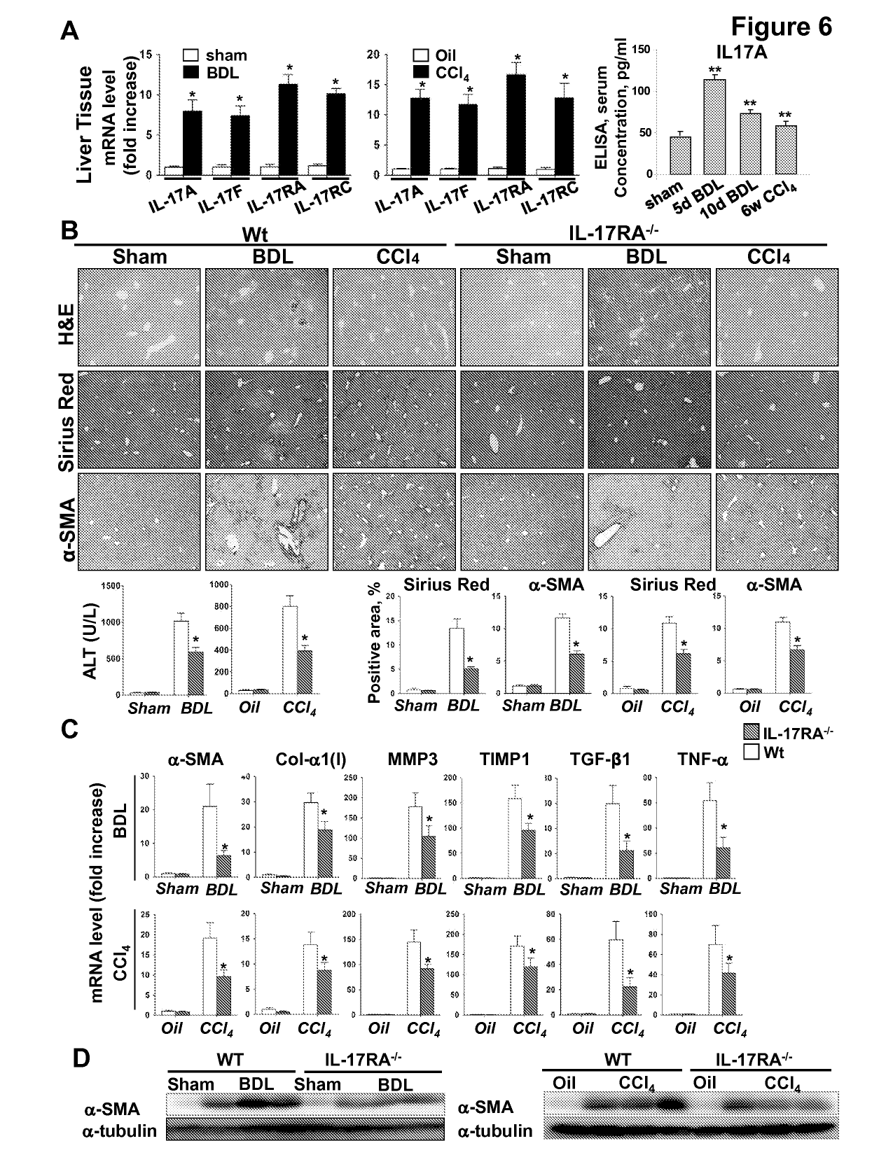

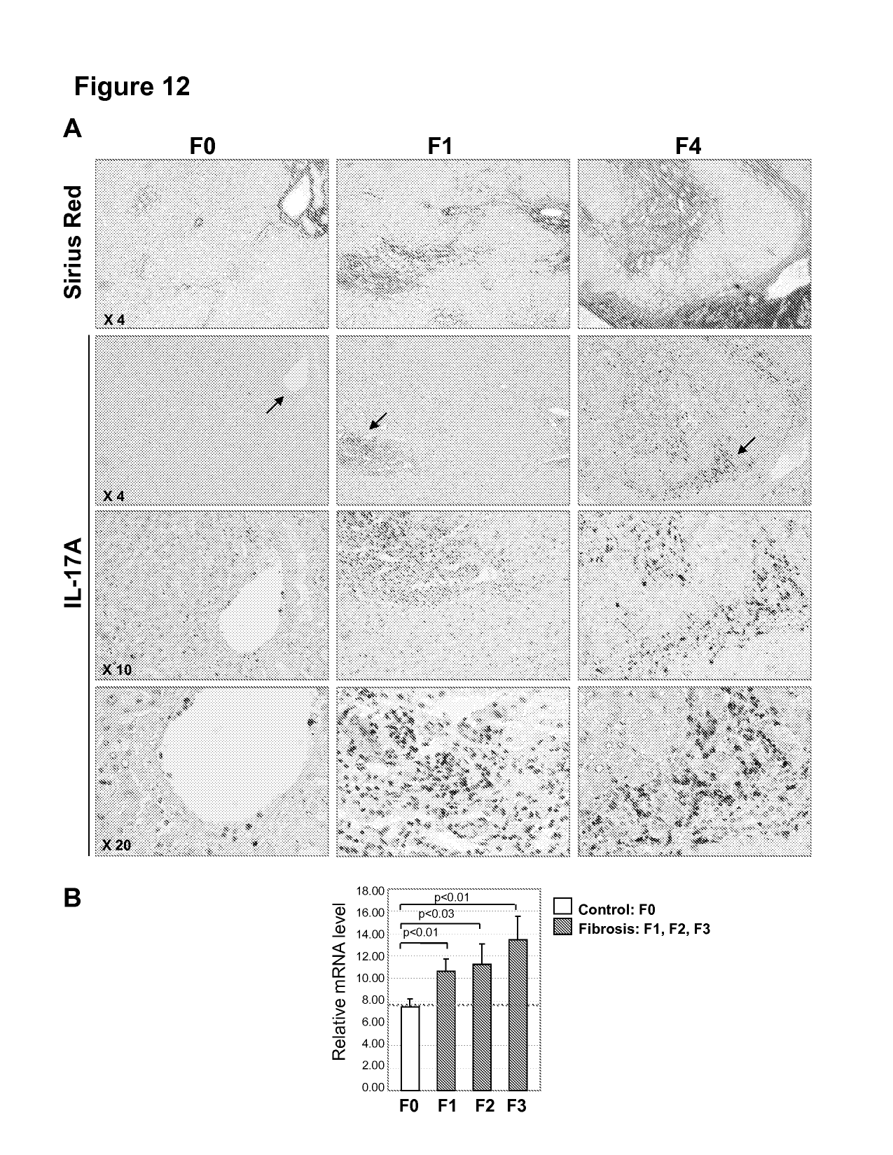

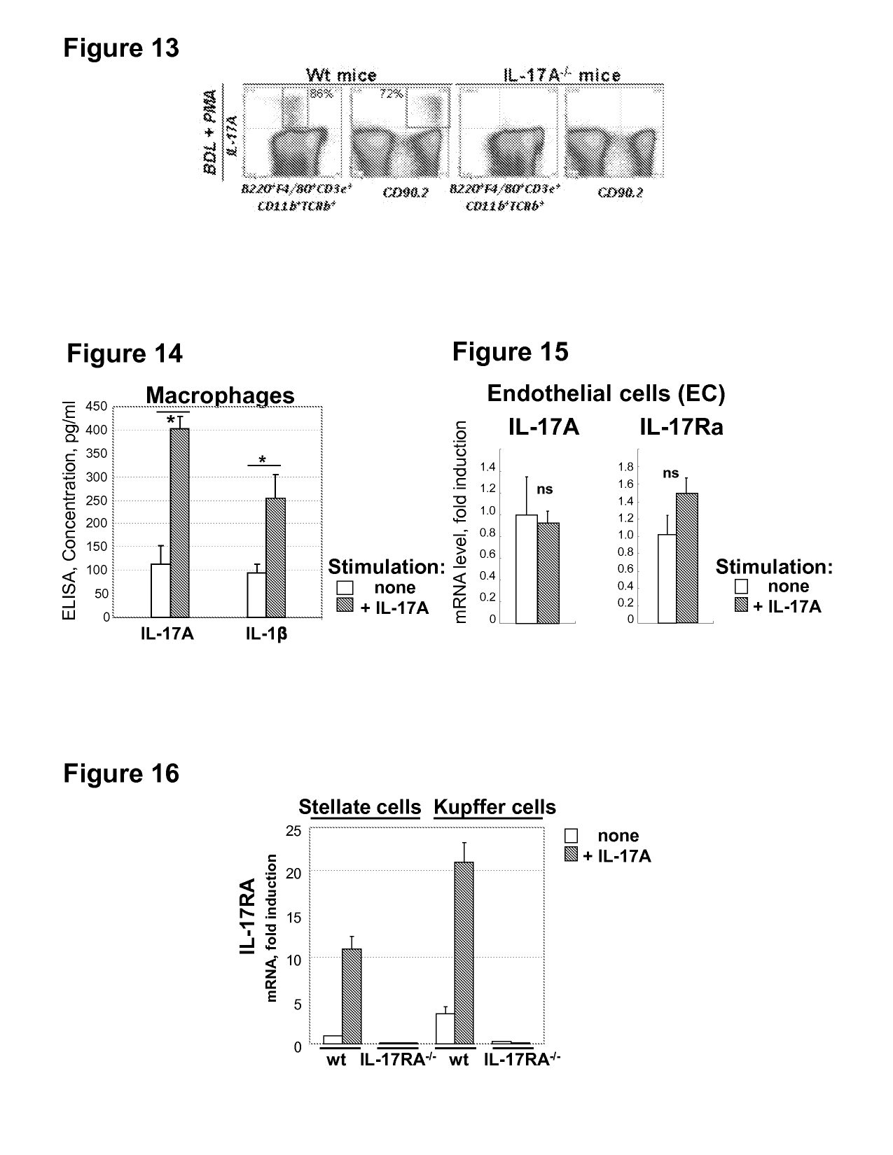

Expression of IL-17A and IL-17F and their cognate receptors IL-17RA and IL-17RC was examined in two models of liver fibrosis in mice: BDL and CCl₄. We determined that mRNA levels of IL-17A, IL-17F, IL-17RA and IL-17RC in fibrotic livers were strongly upregulated independent of the etiology of fibrosis (FIG. 6A). Development of liver fibrosis was also associated high levels of circulating IL-17A (FIG. 6A). Moreover, increased expression of IL-17A was detected in livers from patients with liver fibrosis and cirrhosis of different etiology (vs patients with no fibrosis), and correlated with the severity of the disease (FIG. 12). We conclude that IL-17 signaling may contribute to the pathogenesis of liver fibrosis.

### Example 3

### IL-17RA^{-/-} Mice are Resistant to Liver Fibrosis

The role of IL-17 signaling in hepatic fibrosis was studied in IL-17RA^{-/-} mice, subjected to BDL or CCl₄ (FIG. 6B-D). BDL-induced liver fibrosis was inhibited in IL-17RA^{-/-} mice, as demonstrated in IL-17RA^{-/-} mice by a decrease of collagen deposition (4±1% positive area) and the number of α-SMA⁺ myofibroblasts (5±1.5%) compared to wild type mice (13±4% and 12±1%, respectively; FIG. 6B). The liver function was also improved in IL-17RA^{-/-} mice (FIG. 6B). Reduced mRNA expression of fibrogenic genes (α-SMA, Col-α1(I), MMP3, TIMP1, TGF-β1 and TNF-α, FIG. 6C) in livers of BDL-operated IL-17RA^{-/-} mice correlated with low levels of α-SMA protein (vs wild type mice; FIG. 6D). Similar results were obtained in CCl₄-injured IL-17RA^{-/-} mice (FIG. 6B-D), suggesting that ablation of IL-17 signaling significantly attenuates development of liver fibrosis of different etiologies in mice.

### Example 4

### CD45^{±}TCRβ⁺ Cells are the Major Source of IL-17a in Fibrotic Liver in Mice

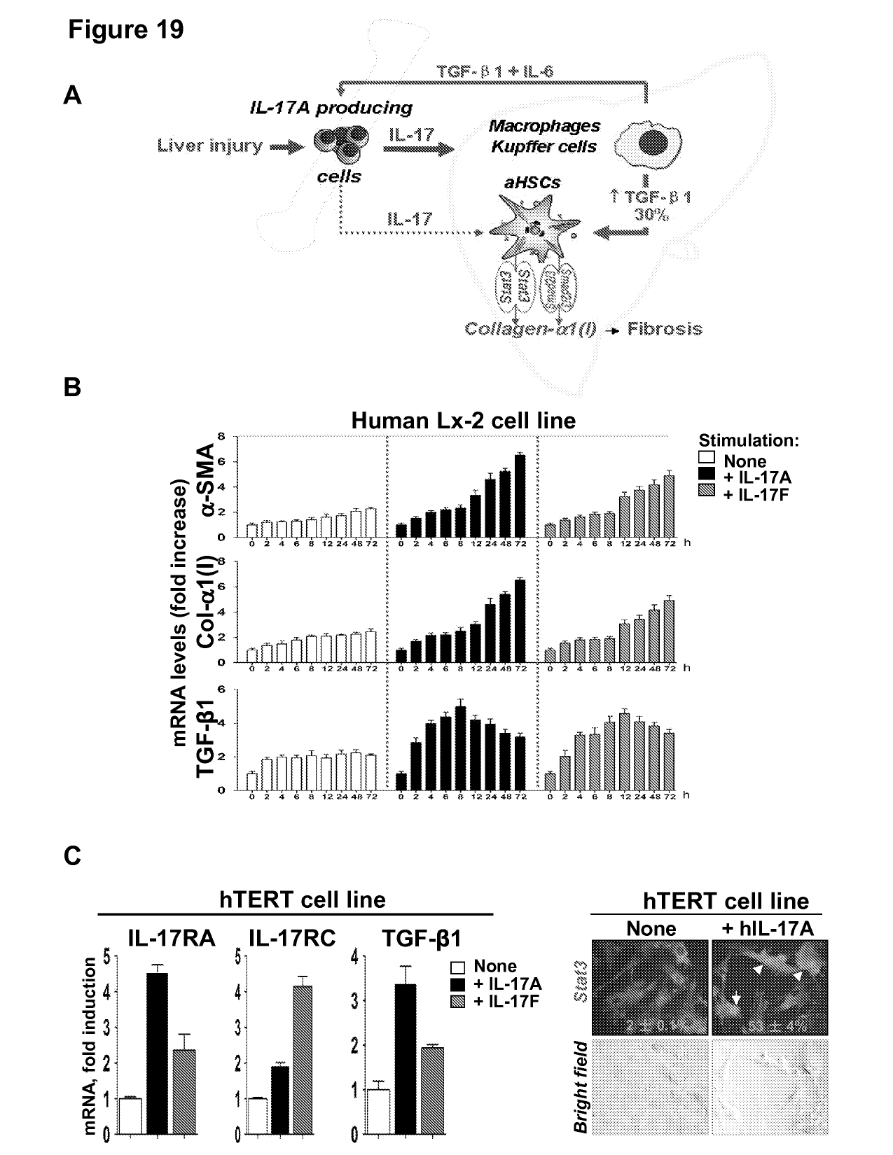

To identify the source of IL-17 in fibrotic livers, the lymphoid fraction² was isolated from livers of sham- or BDL-operated wild type mice, in vitro stimulated with known activators of IL-17, phorbol-miristate acetate (PMA) plus Ionomycin or IL-23⁵, and analyzed by flow cytometry for intracellular IL-17A and IL-17F expression. Following PMA/Ionomycin stimulation, the number of IL-17A-expressing cells was 12 fold increased in livers of BDL wild type mice versus sham controls (FIG. 7A, FIG. 19). In comparison, the number of IL-17F⁺ cells was 2.3 fold increased under similar conditions. Both, PMA/Ionomycin and IL-23 induced mostly production of IL-17A homodimers (over IL-17F homodimers, in ratio 6:1 and 3:1 respectively), suggesting that IL-17A plays a more significant role in liver fibrosis than IL-17F. PMA/Ionomycin-induced IL-17A⁺ cells expressed predominantly T cell markers CD3 (83±4%), TCRβ (74±2%) and TCRγδ (21±5%). Similar results were obtained in IL-23-stimulated IL-17A⁺ cells, demonstrating that T cells are the major source of IL-17A in fibrotic livers.

### Example 5

### Expression of IL-17 Cytokines/Receptors by Liver Resident Cells

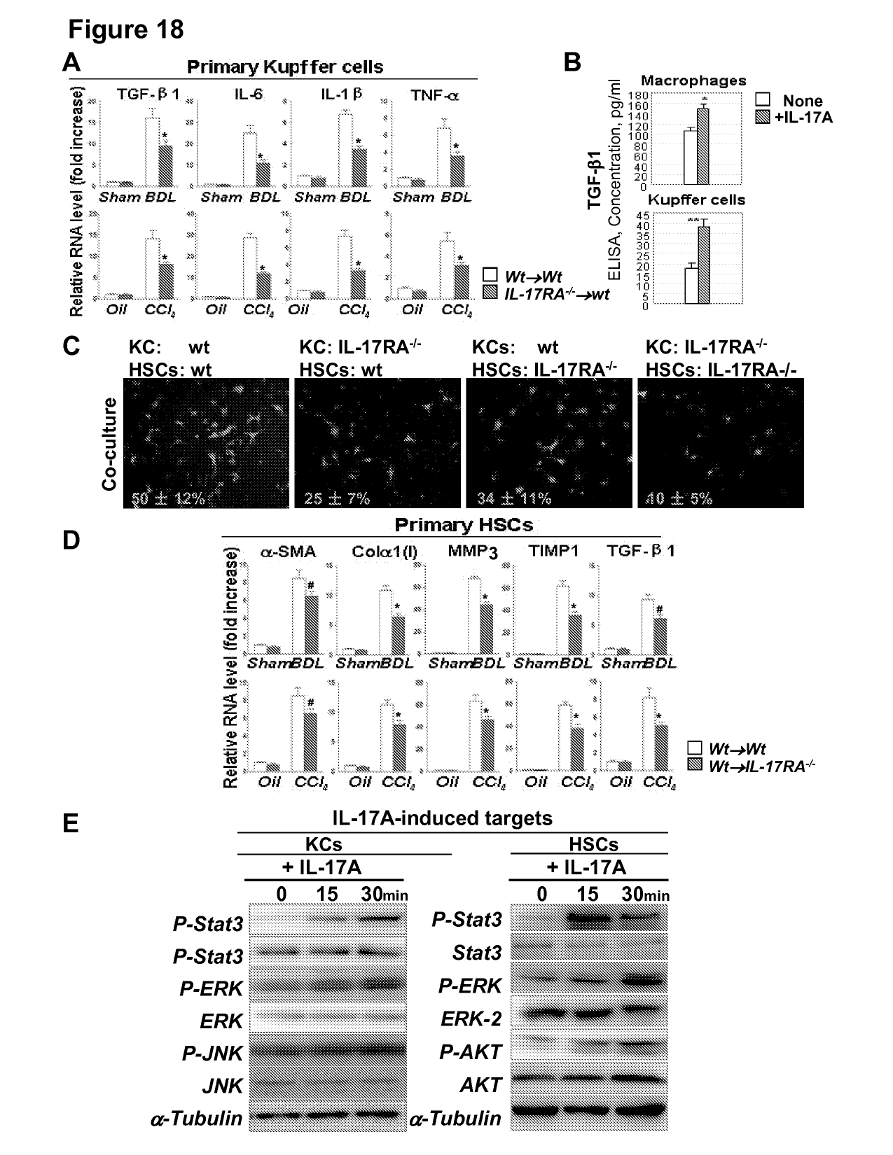

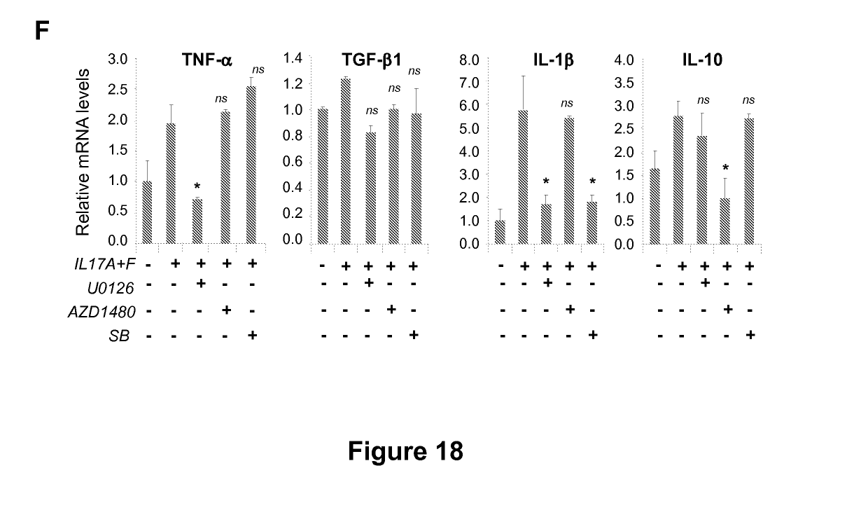

Hematopoietic cells respond to IL-17A stimulation by upregulation IL-17 production⁵. We next examined the ability of liver resident cells to produce IL-17 and transmit IL-17 signals. Primary mouse KC, hepatocytes, quiescent and activated HSCs, and endothelial cells (EC) were isolated from wild type mice, and their responses to IL-17 were evaluated in culture (FIG. 7B). Only KC express and further upregulate IL-17A, IL-17F and IL-1β in response to stimulation with IL-17 (IL-17A, IL-17F or a combination of both cytokines) as demonstrated by RT-PCR (FIG. 7B) and ELISA (FIG. 18). Although the mRNAs for IL-17RA and IL-17RC were expressed by hepatocytes, KC, HSCs (FIG. 7B), and EC (FIG. 15), only KC and HSCs responded to IL-17 by increasing the mRNAs of IL-17RA and IL-17RC, and expression of fibrogenic cytokines TGF-β1, IL-6, and TNF-α (FIG. 7B and FIG. 16). Thus, in addition to BM-derived T cells, KC may also contribute to local IL-17A production and IL-17-induced secretion of pro-fibrogenic cytokines. Consistent with previous findings, hepatocytes²⁰ and EC²¹ do not transmit IL-17 signals despite IL-17RA expression.

### Example 6

### Deletion of IL-17 Signaling in Inflammatory and KC Results in Inhibition of Liver Fibrosis

To assess IL-17 signaling in inflammatory+KC, wild type mice were pre-treated with clodronate (to deplete KC)², lethally irradiated, and reconstituted with IL-17A^{-/-} or IL-17RA^{-/-} BM to generate IL-17A^{-/-}→wt or IL-17RA^{-/-}→wt mice, respectively (FIG. 17). After a two month recovery, liver fibrosis was induced in these mice by BDL. In comparison with wild type (Wt→wt mice, 100%), deficiency of either IL-17A or IL-17RA in BM cells decreased liver fibrosis by 50±4% or 55±6% (versus 75±5% in IL-17RA^{-/-}→IL-17RA^{-/-} mice) (FIGS. 8A-B) Inhibition of fibrosis correlated with lower mRNA levels of fibrogenic genes (α-SMA, Col-α1(I), MMP3, TIMP1, TGF-β1 and TNF-α, FIG. 8C), and lower α-SMA (FIG. 8D), demonstrating that IL-17 signaling in inflammatory cells+KC contribute to the pathogenesis of liver fibrosis. TGF-β1 plays a critical role in development of liver fibrosis, and our data suggest that IL-17 signaling induces ≈30% of TGF-β1 production by BM-derived inflammatory cells+KC in fibrotic livers (FIG. 8C).

### Example 7

### Deletion of IL-17 Signaling in Non-Hematopoietic Endogenous Liver Cells Attenuates Hepatic Fibrosis

The importance of IL-17 signaling in HSCs (and other non-inflammatory liver resident cells) in liver fibrosis was tested in chimeric Wt→IL-17RA^{-/-} mice. In comparison with Wt→wt mice (100%), activation of α-SMA⁺ myofibroblasts and total collagen deposition was attenuated in Wt→IL-17RA^{-/-} mice (25±7% inhibition vs 69±5% in IL-17RA^{-/-}→IL-17RA^{-/-} mice), suggesting that IL-17 signaling of liver resident (non-inflammatory) cells contributes to fibrosis. Since, hepatocytes²⁰ and EC²¹ did not respond to IL-17 signaling to facilitate fibrogenesis (FIG. 7B, FIG. 15), we conclude that ablation of IL-17 signaling in HSCs was critical for inhibition of liver fibrosis in Wt→IL-17RA^{-/-} mice.

### Example 8

### IL-17RA-Deficient KC and HSCs Exhibit Impaired Activation In Vivo in Response to Liver Injury

We compared purified KC/macrophages from the fibrotic livers of chimeric IL-17RA^{-/-}→wt mice with wt→wt mice. We detected decreased mRNA levels of TGF-β1 (↓7 fold), IL-6 (↓14 fold), IL-1β (↓4 fold), and TNF-α (↓3 fold) in IL-17RA^{-/-} KC (vs wild type cells, FIG. 18A). Furthermore, IL-17A stimulates TGF-β1 production in KC/macrophages (FIG. 18B). In co-culture experiments, IL-17A-stimulated wild type KC activated wild type and IL-17RA-deficient HSCs more effectively than IL-17RA^{-/-} KC (FIG. 18C).

We next compared expression of fibrogenic genes in in vivo activated primary IL-17RA-deficient and wild type HSCs prepared from Wt→IL-17RA^{-/-} and Wt→Wt mice, respectively (FIG. 18D). BDL-activated IL-17RA^{-/-} HSCs expressed less α-SMA (↓3 fold), Col-α1(I) (↓5 fold), MMP3 (↓22 fold), TIMP1 (↓24 fold), and TGF-β1(↓3 fold) mRNA (vs wild type HSCs). Similar results were obtained in CCl₄-activated HSCs (FIG. 18D). Thus, despite an intact IL-17 pathway in BM cells in Wt→IL-17RA^{-/-} mice, the in vivo activation of IL-17RA-deficient HSCs into myofibroblasts is impaired. Thus, we hypothesized that IL-17 may directly regulate HSC activation.

### Example 9

### Activation of Target Genes in IL-17A Stimulated KC and HSCs

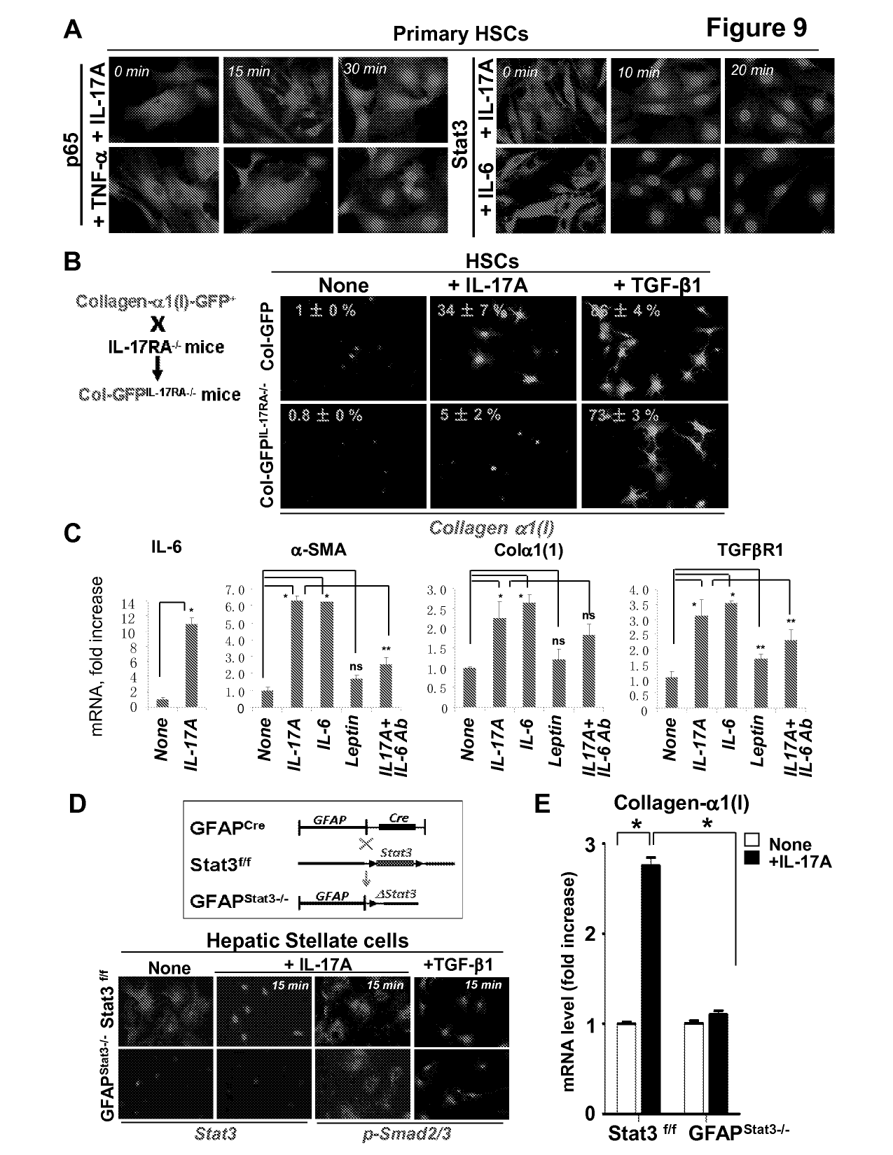

Next, phosphorylation of IL-17 target proteins in KC and HSCs was assessed in vitro (FIG. 18E). Stimulation of primary wild type KC with IL-17A resulted in phosphorylation (within 30 min) of Stat3, ERK, and JNK. Similarly, phosphorylation of ERK and AKT but not JNK (not shown) was observed in primary wild type HSCs after IL-17A treatment (30 min). In particular, strong and rapid phosphorylation of Stat3 in HSCs was detected 15 min after IL-17A treatment (FIG. 18E). While the IL-17 signaling pathway in macrophages is characterized^{8,22} (and FIG. 18F), IL-17 signaling in HSCs is unknown. Similar to TNF-α, IL-17A induced nuclear translocation of NFκB p65 in HSCs after 30 min of treatment (FIG. 9A). Similar to IL-6, we observed a rapid (within 10-15 min) nuclear translocation of Stat3 in HSCs in response to IL-17A treatment (FIG. 9A). Thus, similar to macropahges^{8,22}, NF-κB and Stat3 are downstream targets of IL-17 signaling in HSCs. While NF-κB signaling is required for survival of activated HSC²³, Stat3 may mediate HSC activation by stimulating collagen production.

### Example 10

### IL-17 Stimulates Collagen Production in HSCs Via Stat3 Activation

The ability of IL-17A to induce HSC activation into myofibroblasts was assessed in vitro. For this purpose, primary HSCs were isolated from Col-GFP reporter mice (expressing GFP under collagen-α1(I) promoter) or Col-GFP^{IL-17RA-/-} mice (generated by crossing IL-17RA^{-/-} mice with Col-GFP mice, FIG. 9B). IL-17A stimulation increased expression of GFP in wild type HSCs from a baseline of 1±0% to 34±7%, but not in IL-17RA^{-/-} HSCs (5±2%). As a control, expression of collagen-α1(I)-GFP was strongly induced in both wild type and IL-17RA^{-/-} HSCs by the fibrogenic cytokine TGF-β1 (FIG. 9B). Interestingly, the pro-fibrogenic activity of IL-17A on HSCs is comparable to IL-6 (but not leptin), and is only in part depends on autocrine IL-17-induced IL-6 production (FIG. 9C). Depletion of IL-6 from IL-17A-stimulated HSCs cultures with anti-IL-6 antibody slightly reduced but did not eliminate HSC activation. Thus, studies in the chimeric mice and in cultured HSCs suggest a direct effect of IL-17A on activation of HSCs.

HSCs are the only liver cell type that expresses GFAP¹. The role of Stat3 in IL-17A-mediated collagen-α1(I) expression was tested in primary wild type HSCs and Stat3-deficient HSCs, isolated from GFAP^{Stat3-/-} mice (GFAP^{Cre} mice×floxed Stat3^{f/f} mice, FIG. 9D). IL-17A triggered nuclear localization of phosphorylated Stat3 in wild type HSCs, but not in Stat3-deleted HSCs (FIG. 9D). IL-17A stimulation of wild type HSCs induced collagen-α1(I) expression (FIG. 9E). Meanwhile, IL-17A did not induce nuclear localization of p-Smad2/3 (FIG. 9D), suggested that compensatory cross-activation of the TGFβ1-Smad2/3 signaling pathway is not responsible for stimulating collagen-α1(I) expression in wild type HSCs. In contrast, IL-17A failed to induce collagen-α1(I) expression in Stat3-deficient HSCs (FIG. 9E), demonstrating the requirement of Stat3 in IL-17A-induced fibrogenic activation of HSCs (summarized in FIG. 19A). Next, the relevance of IL-17 signaling was accessed in human HSC cell lines. The LX-2¹² cell line responded to IL-17A and IL-17F stimulation by strong induction of fibrogenic genes α-SMA (↑6 fold), Col-α1(I) (↑7 fold) and TGF-β1 (↑3-5 fold) (FIG. 19B). Similarly, IL-17A and IL-17F mediated activation of the hTERT cell line¹³, and this effect was associated with nuclear translocation of Stat3 (FIG. 19C).

### Example 11

### Stat3 Signaling Facilitates Fibrogenesis in HSCs

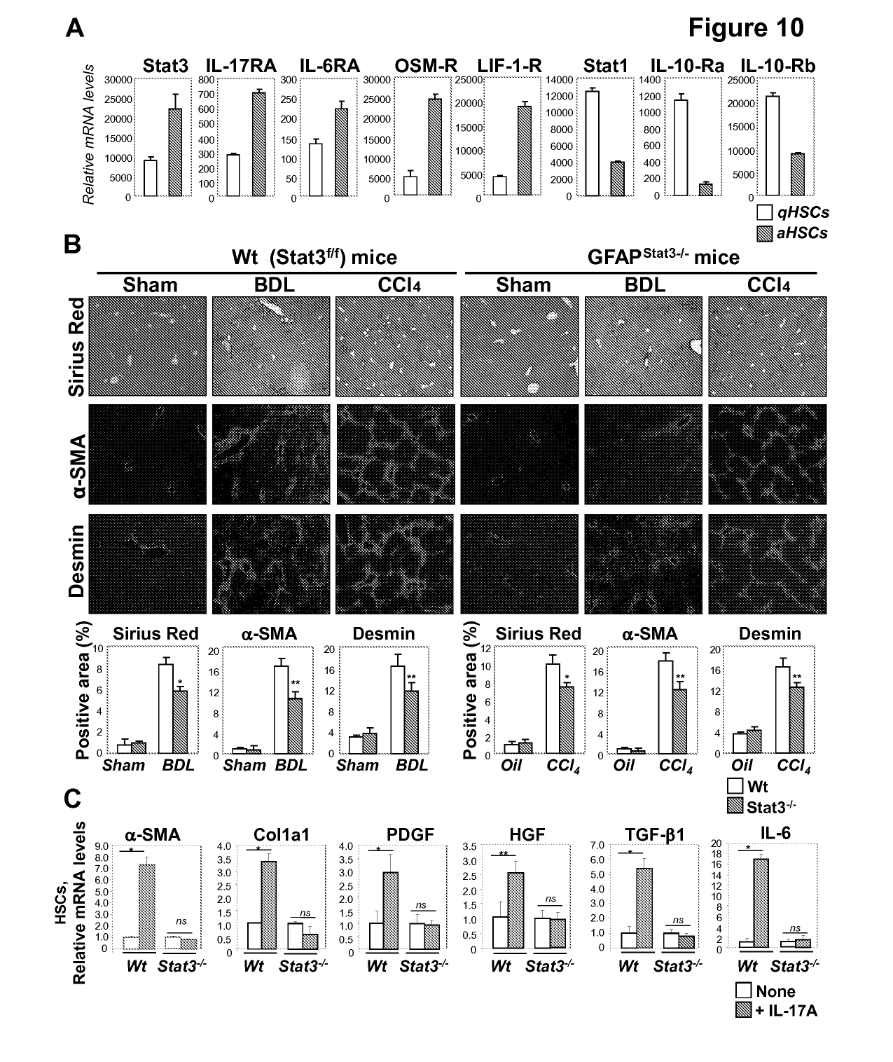

Stat3-signaling cytokines, such as leptin, IL-6, EGF, HGF and PDGF, may have a direct effect on HSC activation, including by increasing collagen production²⁴. Moreover, analysis of the gene expression profile of aHSCs revealed upregulation of Stat3 and Stat3-signaling cytokine receptors (IL-17RA, IL-6RA, OSM-R, LIF-1-R) in comparison with qHSCs (FIG. 10A), consistent with a role for Stat3 in HSC activation. In concordance, GFAP^{Stat3-/-} mice, that are selectively Stat3 deficient in HSCs, had less BDL- or CCL₄-induced liver fibrosis than the wild type Stat3^{f/f} littermates (FIG. 10B). Thus, our data demonstrate that activation of the Stat3 signaling pathway in HSCs has a pro-fibrogenic effect. In concordance, not only Stat3−/− HSCs abrogated collagen and aSMA expression in response to IL-17A, but also downregulated expression of pro-fibrogenic cytokines in primary HSCs (FIG. 10C).

### Example 12

### IL-23 and IL-22 Play Opposing Roles in Liver Fibrosis

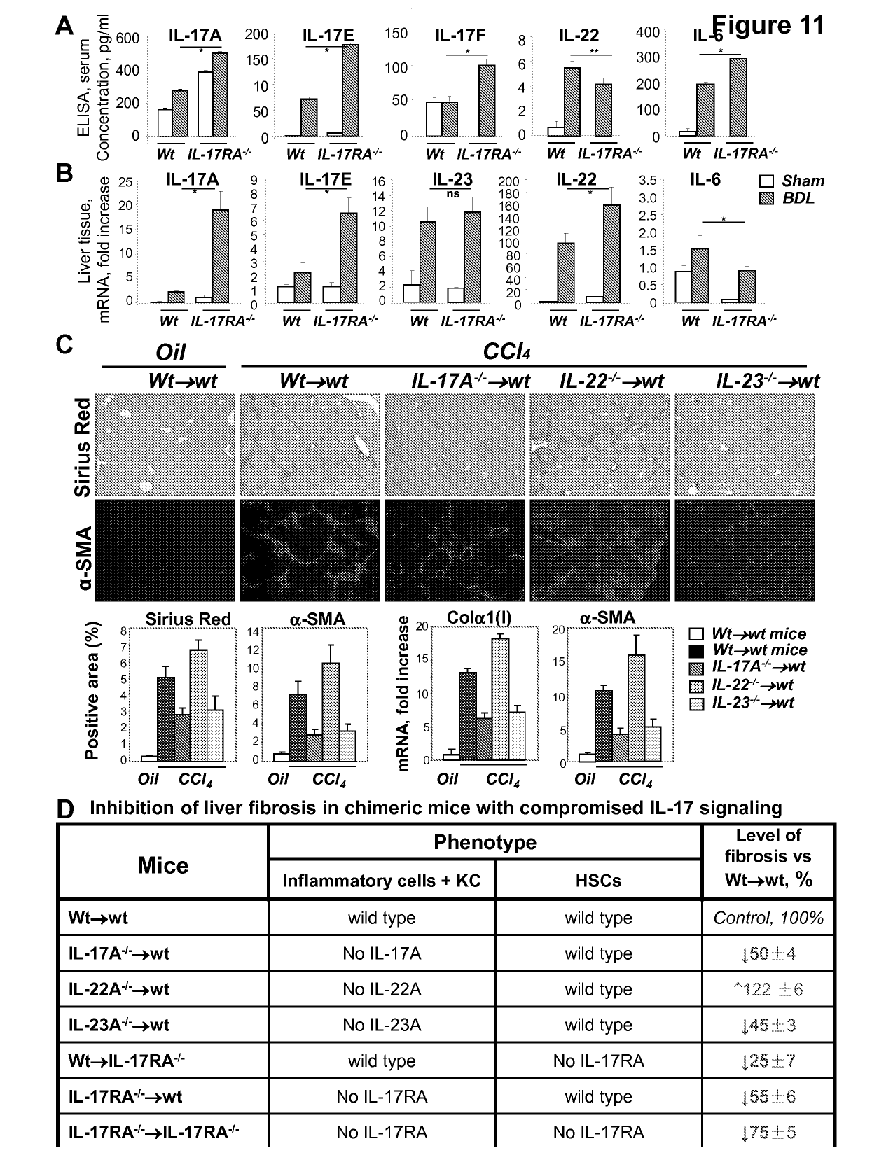

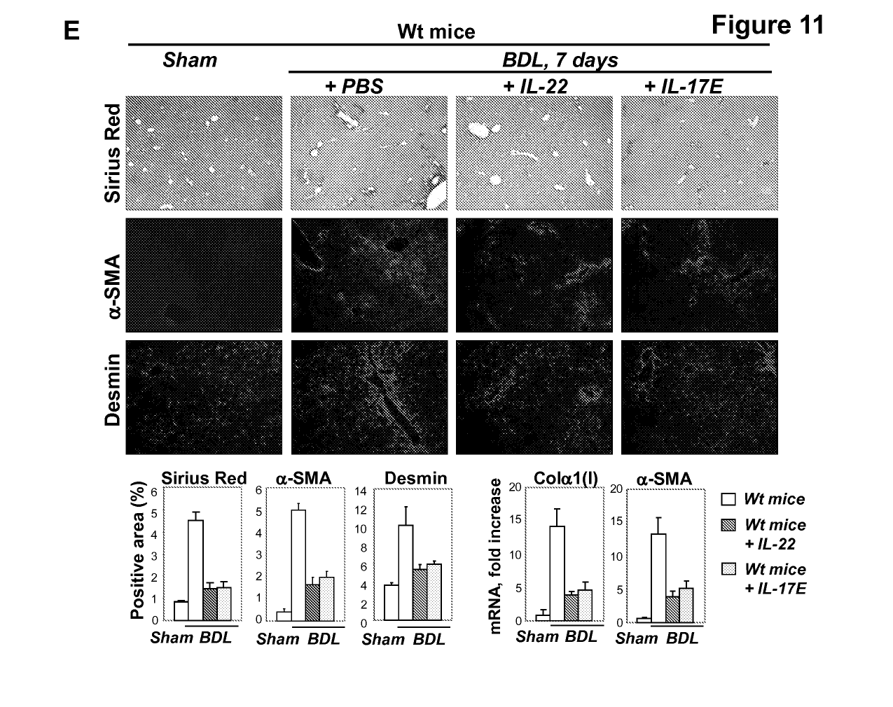

Deletion of IL-17RA in mice results in dysregulation of the IL-17 signaling pathway²⁵. We further evaluated the mechanism of IL-17 signaling in liver fibrosis and compared production of of IL-17A, IL-17F, IL-17E, IL-22 and IL-6 in wild type and IL-17RA^{-/-} mice. Despite elevated levels of circulating IL-17A, IL-17F, IL-6, and IL-17E in the serum of IL-17RA^{-/-} mice (FIG. 11A), deletion of IL-17RA resulted in attenuated liver fibrosis (FIG. 6B), and was associated with a reduction of the pro-fibrogenic cytokine IL-6 mRNA expression in the liver, and the simultaneous upregulation of hepatoprotective cytokine IL-22 mRNA, and IL-17E mRNA (FIG. 11B). In concordance, deletion of IL-23 in the BM of IL-23^{-/-}→wt mice attenuated liver fibrosis to a level similar in IL-17A^{-/-}→wt mice, while deletion of IL-22 significantly exacerbated CCl₄-induced liver fibrosis in chimeric BMT IL-22^{-/-}→wt mice, suggesting that IL-23 and IL-22 produce opposing effects on liver fibrosis (FIG. 11C). The effect of blocking IL-17 signaling in different chimeric mice on liver fibrosis is summarized in FIG. 11D, demonstrating the role of pro-fibrogenic IL-17A and IL-23 cytokines and anti-fibrogenic IL-22 cytokines in liver fibrosis.

### Example 13

### Administration of IL-22 and IL-17E Decreases Hepatic Fibrosis in Mice

The role of IL-22 and IL-17E in liver fibrosis was examined in BDL-operated mice, injected with IL-22 (0.5 μg/mouse) or IL-17E (0.5 μg/mouse, FIG. 11E). Daily administration of IL-22 and IL-17E significantly attenuated development of liver fibrosis, as demonstrated by reduction of Sirius Red staining (↓3 fold), mRNA for Col-α1(I) (↓3 fold) and α-SMA (↓2.5 fold). Our data suggest that IL-22 and IL-17E produce anti-fibrogenic effect in mice.

### Example 14

### Discussion Of Results in Examples 1-13

Data herein demonstrate that IL-17 plays a critical role in the pathogenesis of cholestatic and hepatotoxic liver fibrosis in mice. IL-17 has a strong pro-fibrogenic effect through two independent mechanisms: First, IL-17 stimulates KC to express inflammatory cytokines IL-6, IL-1β, and TNF-α, as well as the major fibrogenic cytokine TGF-β1. Second, IL-17 directly stimulates HSCs to express collagen Type I and promotes their activation into fibrogenic myofibroblasts via Stat3. IL-17 may serve as an attractive target for anti-fibrotic therapy.

Elevated levels of IL-17A were detected in serum of patients with chronic hepatitis B (CHB), and correlated with increased numbers of circulating Th17 cells and histological manifestations of liver fibrosis^{10,11}. Similar results were obtained in patients with chronic hepatitis C (CHC) in which the development of hepatic fibrosis is accompanied by infiltration of IL-17A⁺ positive cells in the liver.

We studied role of IL-17 in murine models of liver fibrosis using deletions of different components of the IL-17 signaling pathway (FIG. 11D). Complete deletion of IL-17 (IL-17A and F) signaling was achieved in IL-17RA^{-/-} mice, that resulted in ≈75% inhibition of BDL- and CCl₄-induced liver fibrosis (vs wild type mice). In turn, deletion of IL-17RA, or IL-17A in BM and KC caused similar inhibition (≈50-55%) of liver fibrosis, indicating that IL-17A (but not other members of IL-17 family of cytokines²⁶) significantly contribute to the pathogenesis of hepatic fibrosis. Our data are in concordance with previous observations suggesting that 1) IL-17A is required for bleomycin-induced pulmonary fibrosis²⁷ and 2) IL-17A and TGF-β play a cooperative roles in the development of fibrosis.

The critical question is how IL-17A signaling stimulates liver fibrosis. We observed suppression of mRNA levels of cytokines (TGF-β1, IL-6, IL-1β, TNF-α) in livers of IL-17RA^{-/-}→wt mice, indicating that IL-17 signaling in BM and KC is required for inflammatory responses in fibrotic liver. Furthermore, TGF-β1 plays a critical role in pathogenesis of liver fibrosis¹, and is mainly produced by activated KC/macrophages². Here we demonstrate that in vivo activated IL-17RA-deficient KC express ≈30% less TGF-β1 than wild type KC. We speculate that TGF-β1 production may be in part regulated by IL-17A. In agreement, IL-17 was recently identified as a regulator of TGF-β 1 and IL-22 in allograft fibrosis²⁸.

The contribution of IL-17 signaling to liver fibrosis by endogenous liver cells was examined in Wt→IL-17RA^{-/-} BM chimeric mice. Our data revealed that IL-17 signaling in non-inflammatory liver resident cells contributed ≈25% of the liver fibrosis (Table 1). We determined that IL-17A stimulates multiple changes in HSCs, suggesting that HSCs are the critical hepatic cells that transmit IL-17 signals to become myofibroblasts. Although hepatocytes²⁰ and EC²¹ did not to respond to IL-17 signaling, their contribution to local cytokine secretion could not be excluded.

How does IL-17 signaling facilitate HSCs activation into myofibroblasts? Our data demonstrate that stimulation of HSCs with IL-17 results in collagen-α1(I) upregulation via IL-17RA. Moreover, Stat3-deficient HSCs do not upregulate collagen-α1(I) in response to IL-17A, indicating that Stat3 is a required target of IL-17 signaling. In support of this notion, mice deficient of Stat3 in HSCs (GFAP^{Stat3-/-} mice) are less susceptible to liver fibrosis. For the past decade, it was believed that the role of Stat3 in the pathogenesis of liver fibrosis is confined to inflammatory cells and hepatocytes^{24,29}. For example, hepatocyte-specific Stat3 knockout mice demonstrate that IL-6, IL-10, and IL-22 produce hepatoprotective and anti-fibrogenic effects^{20,24}. More recently, Stat3 signaling was linked to activation of HSC/myofibroblasts in response to liver injury³⁰. Thus, leptin promotes the development of liver fibrosis and directly stimulates production of collagen Type I production in HSCs in a Stat3-dependent manner²⁴. Here we demonstrate that IL-17A is another Stat3-signaling cytokine with potent pro-fibrogenic properties, inducing activation of hepatic myofibroblasts directly and via IL-6 secretion.

Deletion of IL-17RA results in dysregulation of a cytokine production in mice, including the upregulatioin of IL-17A²⁵. In concordance, increased levels of circulating IL-17A, IL-17F, and IL-6, and decreased levels of IL-22 were detected in serum of CCl₄-injured IL-17RA^{-/-} mice (compared to the wild type controls). Meanwhile, increased IL-22 and IL-17E levels were detected in CCl₄-injured livers, suggesting that the local levels of hepatic cytokines (versus serum) are critical for pathogenesis of liver fibrosis. While IL-23 regulates expansion of IL-17A-producing T cells^{10,11}, IL-22 was implicated in hepatoprotection⁹. In one embodiment, inhibition of liver fibrosis in IL-17RA^{-/-} mice results from an additive effect of abrogation of IL-17 signals and overproduction of IL-22 (and IL-17E) in the injured liver. In support of this notion, similar to IL-17A→wt mice, deletion of IL-23 in inflammatory cells and KC caused attenuation of liver fibrosis in IL-23→wt mice. In contrast to IL-17A→wt mice, deletion of IL-22 in IL-22→wt mice exacerbated liver fibrosis. Furthermore, administration of IL-22 and IL-17E significantly attenuated development of BDL mice, demonstrating that these cytokines may decrease liver fibrosis.

Here we demonstrate that IL-17 regulates production of TGF-β1 by KC, which in turn, induce activation of HSC into myofibroblasts, and further facilitate differentiation of IL-17 expressing cells. IL-17 directly stimulates collagen expression in HSCs via the Stat3 signaling pathway. In addition, our data suggests that IL-23 and IL-22 have an opposite effect and decrease liver fibrosis.

### Example 15

### Regulation of Th17 differentiation

TGF-β1, IL-6 and IL-21 drive differentiation of Th17 cells from naïve Th0 cells⁴⁹⁻⁵¹ via activation of retinoid-related orphan receptor γt (ROR γt)³⁴. IL-23 is required for Th17 proliferation⁵²⁻⁵⁵. IL-27 antagonizes expansion of Th17 directly via inhibition of IL-23-producing cells. IL-25 also blocks Th17 responses via release of IL-13 which, in turn, suppresses IL-23, IL-1β1 and IL-6 secretion by dendritic (and other) cells^{56,57} (FIG. 1A). In response to alcohol-induced liver injury, Th17 cells release IL-17, which causes induction of IL-8, CXCL1 and recruitment of neutrophils into the liver. Neutrophils facilitate hepatocyte injury and activation of macrophages^{9,44-47}. IL-17 stimulates Kupffer cells to express cytokines IL-6, IL-1β, TNF-α and TGF-β1⁵⁸. We have demonstrated that deletion of either IL-17A or IL-17RA in BM derived cells decreases liver fibrosis by 50-55%, and this effect is mediated via regulation of TGF-β1 production, while deletion of IL-17RA in non-immune liver resident cells decreased liver fibrosis by 25%⁵⁸. Furthermore, we demonstrated that IL-17A directly stimulates activation of HSCs⁵⁸.

### Example 16

### Characterizing the Role of IL-17 in Alcohol-Induced Steatohepatitis

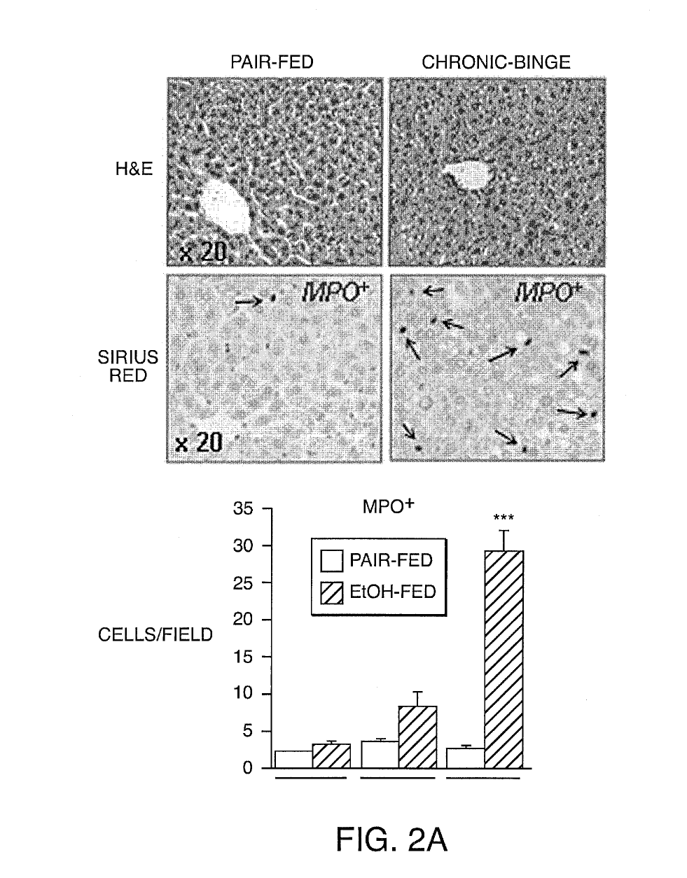

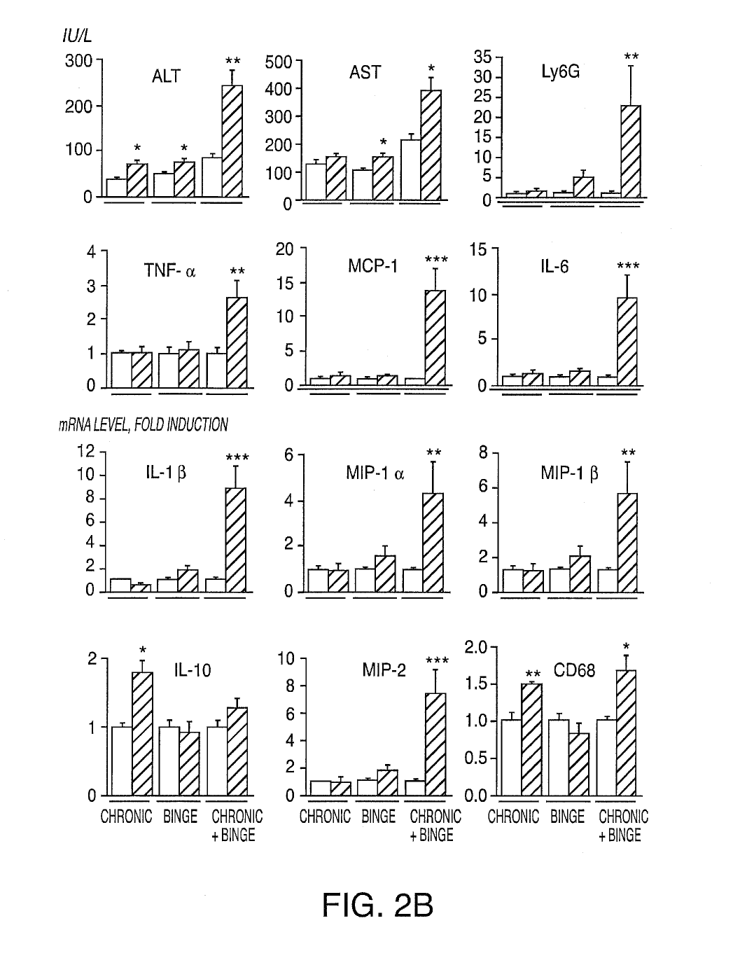

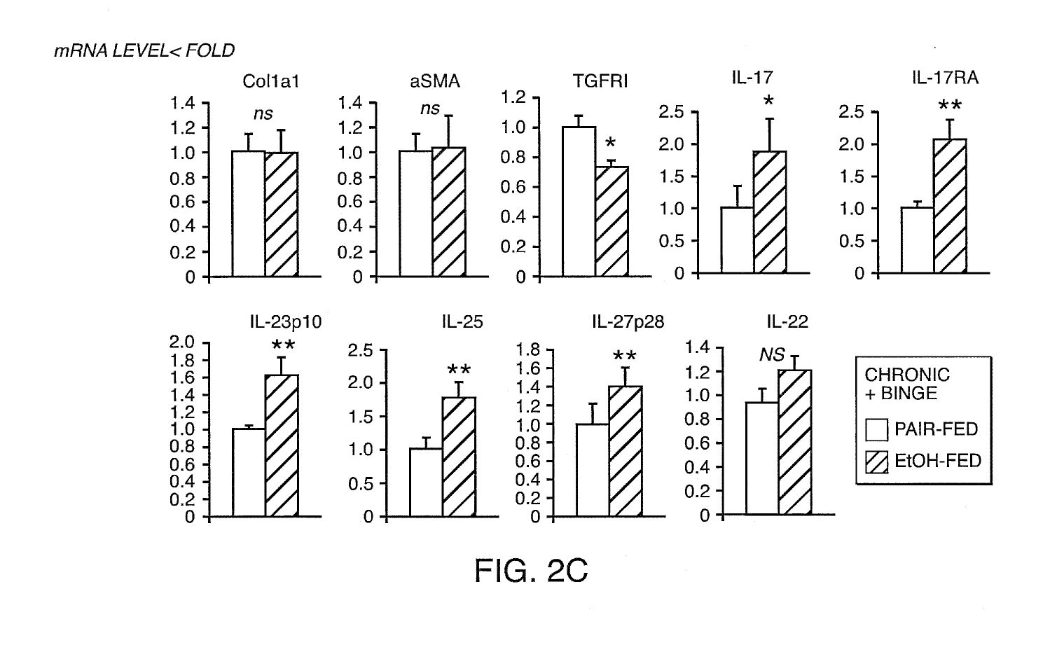

Stetatohepatitis is the first stage of ALD. Patients with steatohepatitis have elevated levels of IL-17⁹, suggesting that IL-17 may play a role in pathogenesis of ALD onset⁸⁶. Chronic-binge alcohol feeding in mice¹² mimics stetatohepatitis in patients. Our data indicate that expression of IL-17 and IL-17RA is upregulated in mice subjected to the chronic-binge model of alcoholic steatohepatitis (FIG. 2). While not intending to limit the invention to any particular mechanism, we hypothesize that in response to chronic alcoholic injury, hepatic IL-17 facilitates recruitment of inflammatory cells to the liver, specifically neutrophils that mediate killing of injured hepatocytes and exacerbate hepatocellular damage. While not intending to limit the invention to any particular mechanism, we also hypothesize that IL-17 not only directly acts as a neutrophil chemoattractant, but also induces production of IL-8 and CXCL1 by other cells to recruit circulating neutrophils in the liver. Using chronic-binge alcohol feeding in wild type¹², IL-17RA^{-/-} and IL-17^{-/-} mice, we will determine the source of IL-17, as well as the target cells responding to IL-17 signaling by inducing hepatic inflammation and injury. Furthermore, we aim to determine the pathways of IL-17 regulation at early stages of alcohol-induced steatohepatitis.

A. Preliminary Results:

C57BL/6 mice (8 w old, n=10) were subjected to choric liver injury (Lieber-DeCarli liquid diet, 10 days), binge alcohol-injury (single gavage with ethanol 5 g/kg of body weight), or chronic-binge alcohol induced injury (Lieber-DeCarli diet+single gavage with ethanol, see Methods). Control age-matched littermates were pair-fed with 9g maltose dextrin/kg of body weight¹². Mice were sacrificed 9 h after the alcohol-binge treatment. The levels of ALT and AST were strongly elevated in chronic-binge injured mice, suggesting that chronic-binge alcohol feeding causes significant damage to hepatocytes (FIG. 2B). Chronic-binge treatment was associated with increased (30 fold) flux of neutrophils to the liver, as demonstrated by immunostaining for neutrophil marker MPO. We observed strong mRNA induction of hepatic cytokines TNF-α, IL-β1, and IL-6 (FIGS. 2A-B). Furthermore, the mRNA levels of MCP-1, MIP-1α and MIP-2 chemokines, which serve as neutrophil chemoattractant, were elevated and correlated with increased (20 fold) expression of neutrophil marker Ly6G in livers. We conclude that chronic-binge model of alcohol feeding mimics alcoholic steatohepatitis similar to that observed in patients, and can be used to study early ALD. In concordance, expression of fibrogenic genes (Col1α1, αSMA, TGFRbI) was not observed in chronic-binge fed mice (FIG. 2C). Meanwhile, mRNA level of hepatic cytokines IL-17 and IL-17RA was moderately upregulated (2 fold), and correlated with the induction of regulatory cytokines IL-23, IL-25 and IL-27 (versus pair-fed mice). Our data suggests that IL-17 signaling may play a role in early development of alcoholic steatohepatitis.

B. Experimental Design:

i) the Role of IL-17 in Alcoholic Steatohepatitis.

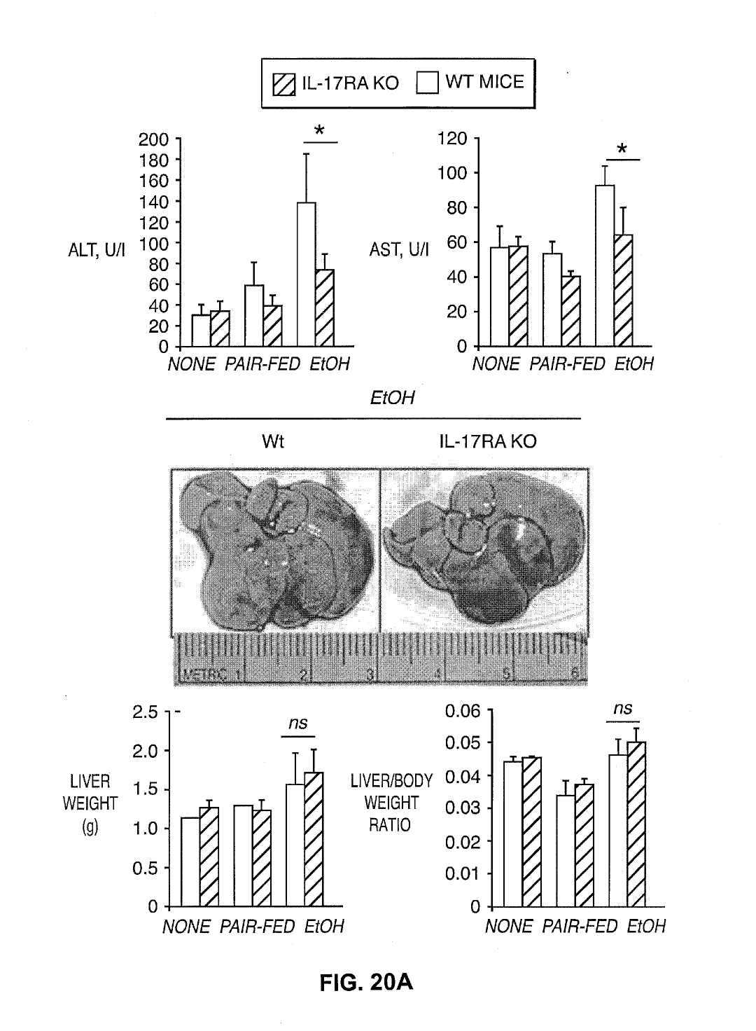

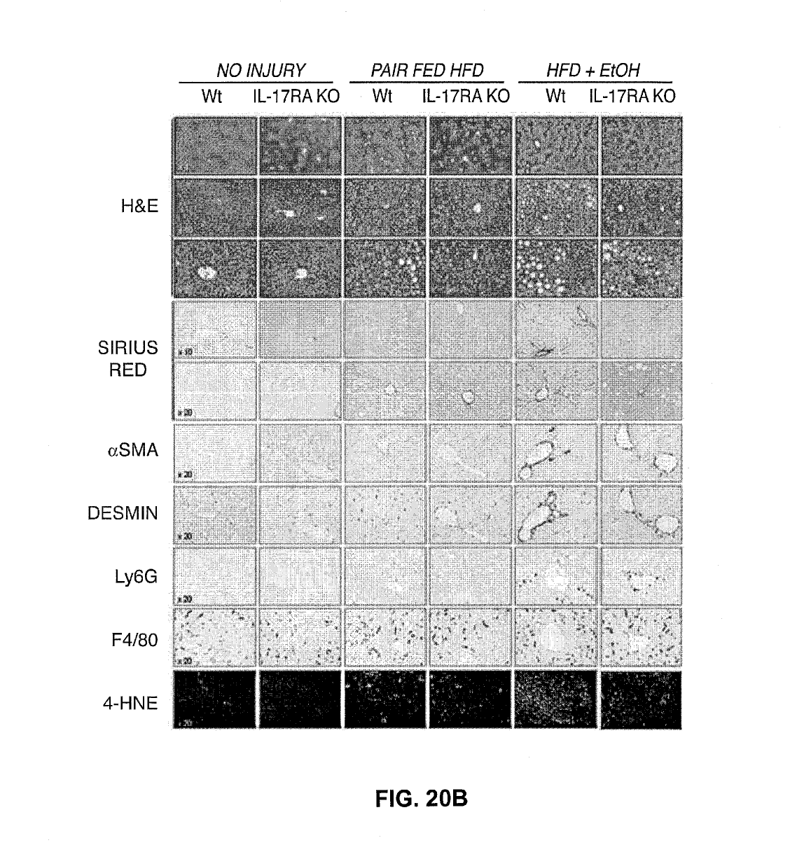

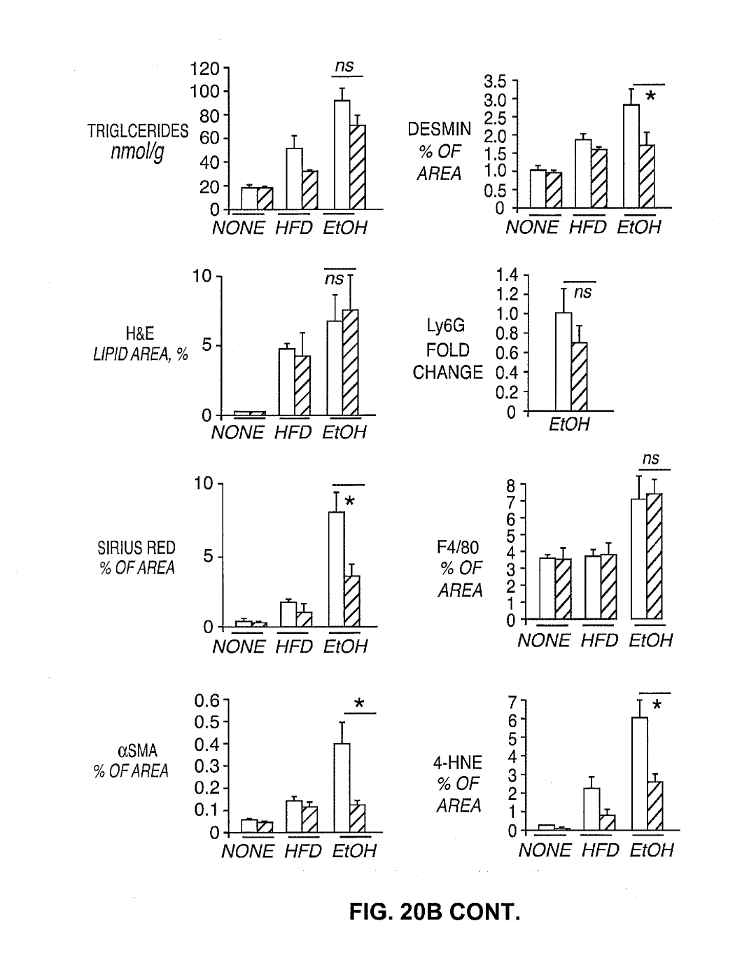

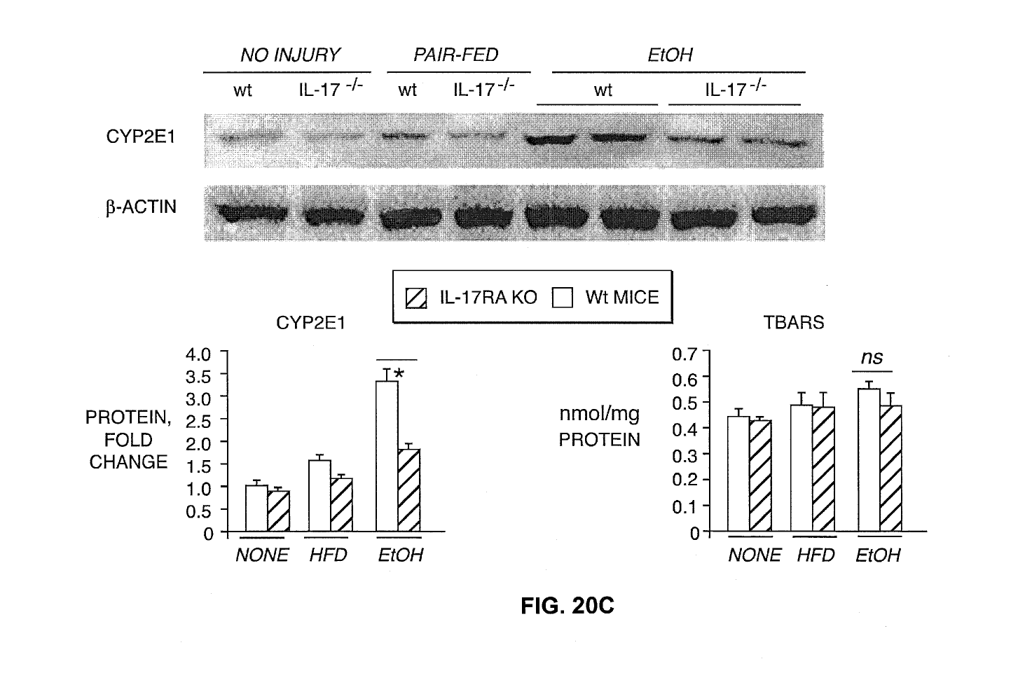

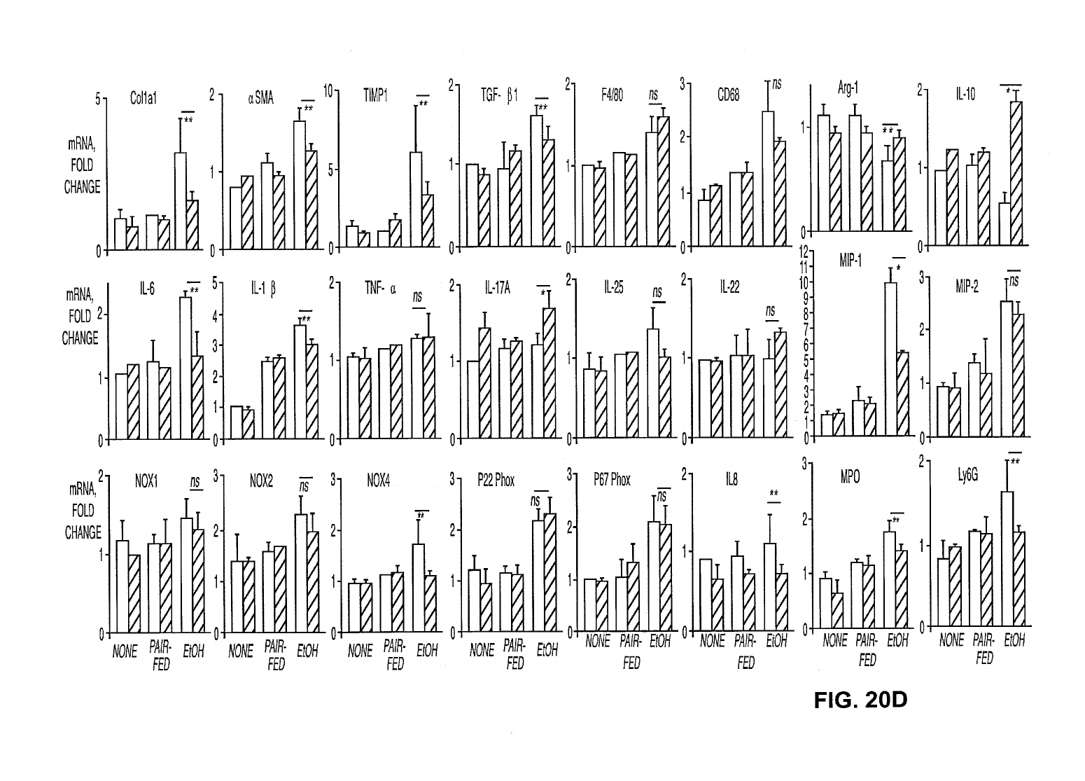

Development of alcoholic steatohepatitis will be evaluated in IL-17RA^{-/-}, IL-17^{-/-} mice and pair-fed wt littermates (males, 8 weeks old, n=10/group, see “Vertebrate Animals”) using a chronic-binge model of alcohol feeding (11 days). Mice are sacrificed 9 hours after binge-treatment and analyzed. While not intending to limit the invention to any particular mechanism, we hypothesize that IL-17 plays a critical role in the release of pro-inflammatory cytokines and recruitment of neutrophils to the injured liver. Data here in FIG. 20 was generated using mice that were fed with high fat diet, high cholesterol, Lieber-Decarli+weakly ethanol binge for 10 weeks. This model mimics earlier stages of alcoholic liver disease-steatohepatitis. Data in FIG. 20 demonstrate that IL-17 signaling plays a critical role in pathogenesis of Alcoholic Liver disease. Development of steatosis, fibrosis and inflammation was attenuated in IL-17RA−/− mice. The data show that IL-17 promotes progression of alcoholic liver disease from steatohepatitis to fibrosis and cancer (hepatocellular carcinoma).

Tissue Analysis:

A) Liver injury is evaluated by measuring serum ALT and AST. Tissue architecture is assessed by H&E and Oil Red O staining. B) The blood alcohol content, level of alcohol-metabolizing enzymes dehydrogenase (ADH), acetaldehyde dehydrogenase (ALDH), and cytochrome P4502E1 (CYP2E1) are evaluated using qRT-PCR and Western blotting. The activity of these enzymes is measured. C) Hepatic lipid content (triglycerides and cholesterol) is measured. Expression of adipogenic genes (PPARγ, PPARα, CEBP1, Adipor, Adpf1, SREBP-1, PGAT-1, PGAT-2, FASN, ACC-a) is measured using qRT-PCR. D) Hepatic levels of lipid peroxidation (TBARS, induction of 4-NHE and depletion of GSH levels, MDA) and ROS production (Nox1, Nox2, Nox4, P22Phox, P47Phox, P67Phox) is measured¹². E) Deletion of IL-17RA in mice may lead to a compensatory dysregulation of cytokine expression profile. Thus, the level of IL-17 is upregulated in IL-17RA^{-/-} mice⁸⁷. Therefore, hepatic and serum levels of inflammatory cytokines TGF-β1, IL-6, IL-1β, IL-17, IL-22, IL-23, IL-25, IL-27, TNFα, IFNγ and CCL2 are measured by qRT-PCR and ELISA⁵⁸, respectively. The expression level of IL-8, CXCL1, G-CSF, and CXCL12, factors responsible for neutrophil migration) and survival, are determined. To estimate the severity of inflammation, liver sections are stained for CD45, F4/80⁺, CD11b⁺ macrophages and monocytes, CD11c⁺ dendritic cells (DCs), NK1.1⁺ cells, and Lyc6G⁺ and myeloperoxidase⁺ (MPO⁺) neutrophils. Complementary flow cytometry is performed to determine the composition of inflammatory cells in the hepatic non-parenchymal cell fraction^{58,63} isolated using gradient centrifugation from livers of alcohol-fed IL-17RA^{-/-}, IL-17^{-/-}, and wt mice. F) Hepatic neutrophils are analyzed further. Neutrophil activation is associated with upregulation of leukotriene B4 (LTB4), and platelet-activating factor (PAF), of enzymes such as neutrophil elastase, MPO, and matrix metalloproteinases (MMP-9), TNFα and IL-8, CD35, CD11b and CD18, and shedding of leucocyte surface adhesion molecules CD62L^{88,89}. Ly6G⁺ neutrophils are sort purified from livers of IL-17^{-/-} and IL-17RA^{-/-} mice, and their activation status is compared to wt neutrophils. The ability of IL-17 to induce transwell migration⁹⁰ of neutrophils in response to co-culturing with IL-17-secreting CD4⁺ T cells (using IL-17^{-/-} CD4⁺ T cells, as a negative control) is assessed. Similarly, wt, IL-17RA^{-/-} and IL-17^{-/-} macrophages are isolated⁹⁰, and the expression level of inflammatory cytokines is compared.

ii) Identification of Cellular Source of IL-17.

The cellular source of IL-17A in the alcohol-injured liver will be identified. For this purpose, liver inflammatory cells (T cells, γδ T cells, NK, NKT cells, Kupffer cells/macrophages) and liver resident cells (hepatocytes, endothelial cells, and HSCs) will be isolated from wt mice⁵⁸ and analyzed by flow cytometry and qRT-PCR for intracellular IL-17 expression. IL-17-expressing populations are identified by co-staining for IL-17 and CD4, CD3, TCRγδ, Gr-1, CD11b, CD8a, NK1.1 as described^{58,83}. In one embodiment, mainly CD4⁺ T cells express IL-17. The cells from IL-17^{-/-} mice serve as a negative control for IL-17 staining.

iii) Identification of IL-17 Target Cells.

Based on previous studies, IL-17RA is ubiquitously expressed in the liver, and its expression is upregulated in response to IL-17 signaling³³. Although different cell types may respond to IL-17 stimulation in vitro, their in vivo activation may vary with the stage of ALD.

Strategy:

A) First, we will determine which cell types in vivo upregulate IL-17RA expression in the liver of chronic-binge fed wt mice (vs pair-fed wt mice). The expression of IL-17RA will be analyzed by flow cytometry and qRT-PCR in inflammatory cells (T cells, γδ T cells, NK, NKT cells, and Kupffer cells/macrophages) and liver resident cells (hepatocytes, HSCs, endothelial cells and cholangiocytes). In one embodiment, at this stage of ALD IL-17RA expression is induced mostly in Kupffer cells/macrophage and hepatocytes. Since chronic-binge model of liver injury is not associated with fibrosis or myofibroblast activation (FIG. 2C), in one embodiment, HSCs will not upregulate expression of IL-17RA. B) Isolated wt and IL-17RA^{-/-} Kupffer cells (hepatocytes and HSCs) are in vitro stimulated with IL-17, and activation of IL-17 signaling pathway is determined (including TRAF6, Act1, JNK, ERK, phosphorylation and nuclear translocation of NF-κB and STAT3^{37,39}).

iv) Experimental Methods:

### Mice:

We obtained IL-17RA^{-/-} mice, IL-17A^{-/-} mice⁹², and STAT3^{f/f} mice⁹³, IL-23p19^{-/-} mice (Genentech). IL-25^{-/-} mice⁷², IL-27p28^{-/-} mice⁹⁴, and Albumin^{Cre} mice, Lysozyme-Cre mice⁹⁵, and GFAP-Cre mice⁹⁶ are purchased from Jackson laboratories.

### Chronic-Binge Ethanol Feeding Model to Mimic Alcoholic Steatohepatitis (ASH)^{12,78}:

The ethanol group are fed a liquid diet containing 5% ethanol for 10 days, and at day 11 gavaged with a single dose of ethanol (5 g/kg body weight, 20% ethanol) in the morning and sacrificed 9 h post gavage when ALT and AST peak. Control groups are pair-fed control diet for 10 days and gavaged with isocaloric dextrin maltose.

### Isolation of Hepatocytes and Non-Parenchymal Cell Fraction and Primary HSCs:

Livers are perfused using pronase/collagenase method¹⁵. Single-cell suspensions are centrifuged at 50 g for 5 minutes to pellet the hepatocyte fraction. The remaining non-parenchymal cell fraction is collected¹⁵. Kupffer cells and EC are isolated by gradient centrifugation (15% Nycodenz) following by magnetic sorting with anti-CD11b and anti-CD31 antibodies (Miltenyi Biotec). HSCs are isolated using gradient centrifugation (8.2% Nycodenz)¹⁵. HSCs are cultured in Dulbecco's modified Eagle's medium (DMEM; Gibco-BRL) supplemented with 10% fetal calf serum (FCS), 2 mM L-glutamine and standard antibiotics. Hepatocytes are cultured on collagen-coated plastic plates in Waymouth's medium supplemented with 10% FCS.

### Cytochrome P4502E1.

Expression of CYP2E1 is measured in hepatic microsomes⁹⁷ by Western blotting⁹⁸. CYP2E1 activity is measured by the rate of oxidation of p-nitrophenol to p-nitrocatechol⁹⁹.

### Lipid Peroxidation:

Thiobarbituric acid reactive substances formation (OxiSelect™ TBARS Assay Kit, Cell Biolabs) is detected²¹. 4-hydroxy-2-nonenal (HNE) adducts detected by immunostaining of frozen livers using rabbit anti-mouse 4-HNE (Alpha Diagnostic). Malondialdehyde (MDA) and reduced glutathione (GSH) are measured as described^{98,100}.

### Flow Cytometry:

Phenotyping is performed on a BD LSR II (BD) using anti-mouse antibodies (eBioscience). Intracellular staining for IL-17A is performed after cell stimulation with PMA/Ionomycin/Brefeldin A (or IL-23/Brefeldin A)⁵⁸ for 5 hours using BD Cytofix/Cytoperm (BD)⁶³. Immunostaining. is performed on formalin-fixed livers with anti-α-SMA Ab (Dako) using MOM kit followed by DAB staining (Vector). STAT3 (Cell Signaling), NF-κB (p65), phospho-Smad2/3, IL-17A (Santa Cruz) antibodies will be used⁵⁸. The images are taken using confocal microscopy and analyzed by Image J.

### Statistical Analysis.

All data are expressed as means±standard deviation (SD). Differences between experimental and control groups are assessed by two-tailed unpaired Student's t-tests using Graph Pad Prism 5.0 (GraphPad Software, San Diego, Calif.). p-values less than 0.05 are statistically significant.

### Example 17

### Characterizing the Role of IL-17 in Alcohol-Induced Fibrosis

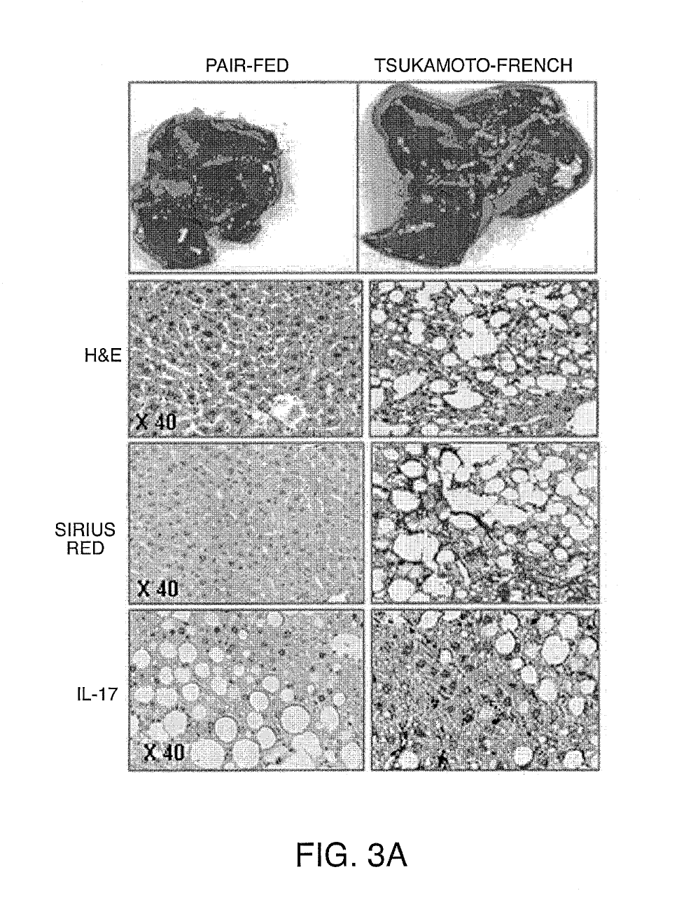

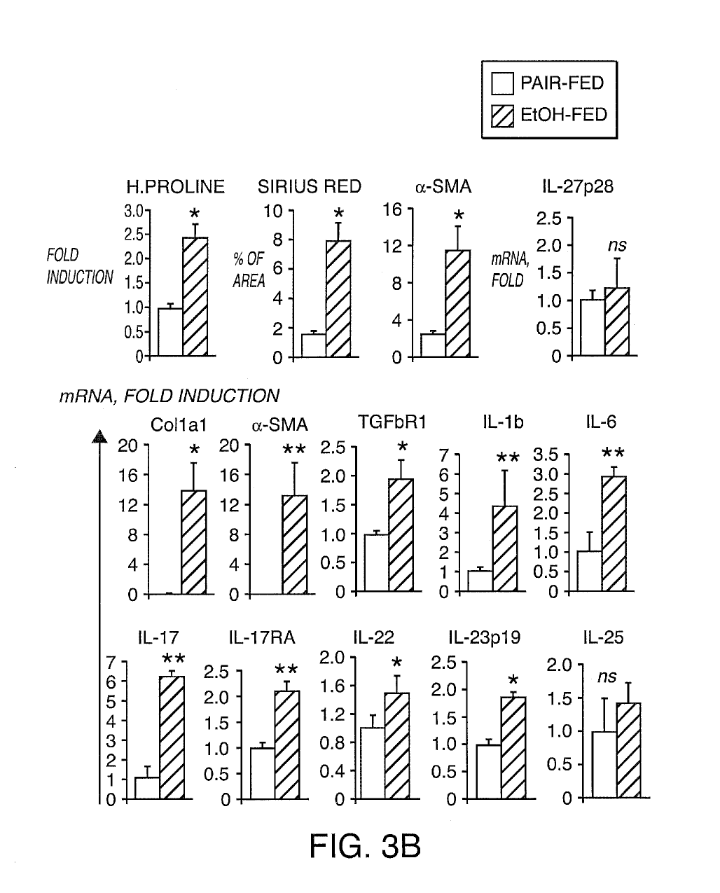

Alcoholic steatohepatitis often progresses to fibrosis, the next stage of ALD. Patients with alcoholic fibrosis have elevated levels of IL-17 that correlate with poor prognosis^{9,86}. The mouse model of intragastric ethanol feeding mimics alcoholic fibrosis in patients^{79,101}. In one embodiment, the role of IL-17 in the pathogenesis of alcoholic fibrosis⁷⁹ is determined. Our studies demonstrate A) that a significant level of liver fibrosis is achieved in C56BL/6 mice in response to intragastric ethanol feeding (FIG. 3). And similar to patients, development of alcoholic liver fibrosis in these mice is associated with increased expression of hepatic IL-17 and IL-17RA. B) Based on our preliminary data, deletion of IL-17RA in mice strongly inhibits development of alcoholic liver fibrosis. While not intending to limit the invention to any particular mechanism, we hypothesize that IL-17 signaling regulates alcohol-induced liver fibrosis via activation of Kupffer cells/macrophages and HSCs.

A. Preliminary Data:

i) Induction of Alcoholic Fibrosis in Wt Mice:

Data herein demonstrate improvements in the intragastric model of ethanol feeding (Tsukamoto-French model)⁷⁹ to achieve a significant level of liver fibrosis in mice after 2 months of alcohol⁸⁰, so that this model most closely reproduces alcoholic fibrosis in patients^{79,81}.

Liver fibrosis was induced in C57BL/6 mice (16 w old, n=10) using the intragastric alcohol-feeding (8 weeks, see methods)^{101-104.} Steatosis was observed in alcohol-fed mice (FIG. 3A). Alcohol-fed mice developed liver fibrosis, as shown by Sirius Red staining and mRNA expression of fibrogenic genes (FIG. 3B). IL-17-producing cells were detected in livers of alcohol-fed mice (vs pair-fed mice). IL-17 (6.5 fold), IL-17RA (2 fold), and IL-23 (2 fold) mRNA expression was induced in these mice (FIG. 3B) and correlated with high levels of IL-6 and IL-1β.

ii) Alcoholic Fibrosis is Reduced in IL-17RA^{-/-} Mice Compared to Wt Mice:

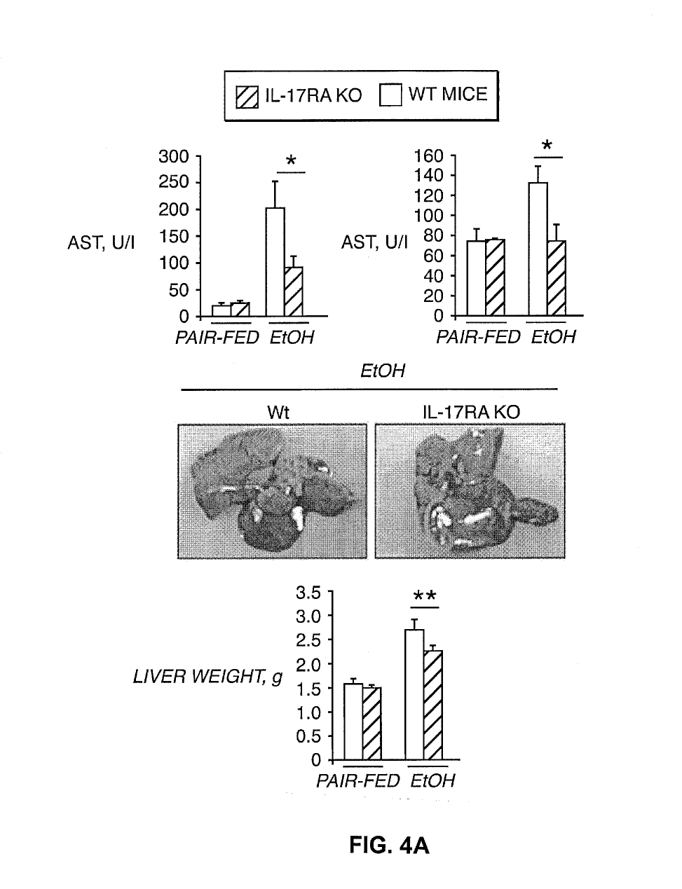

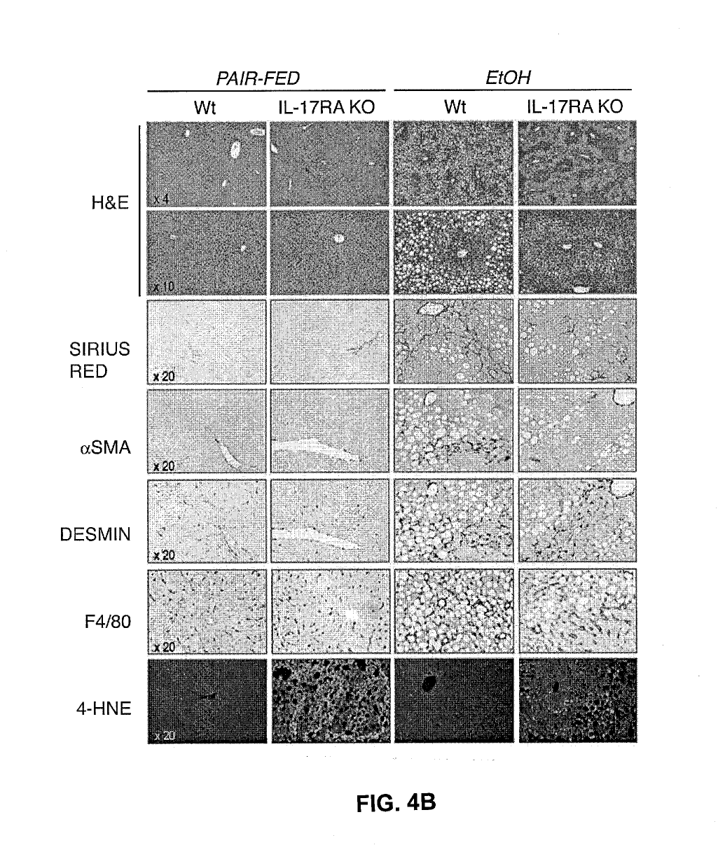

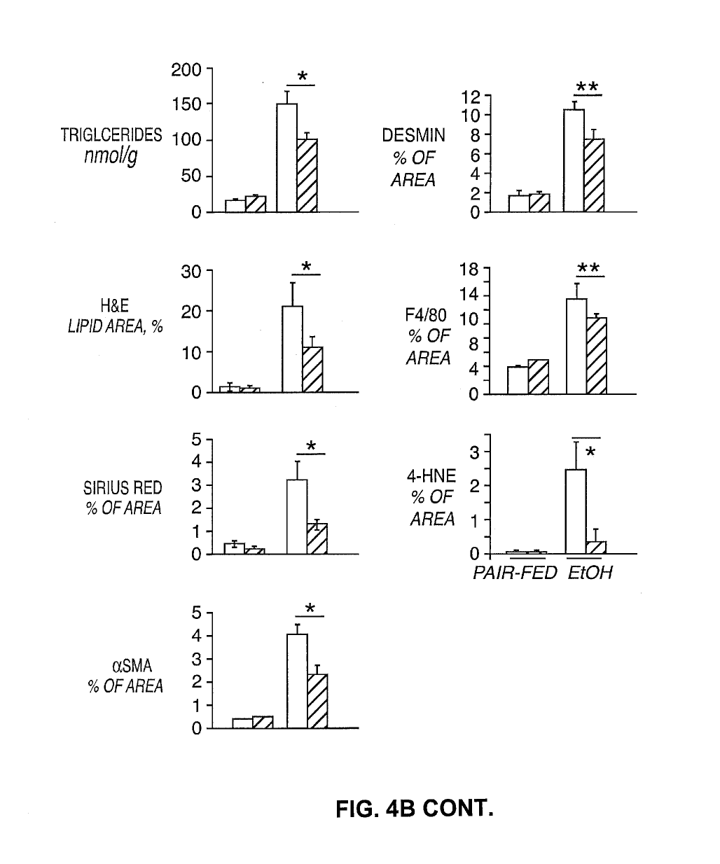

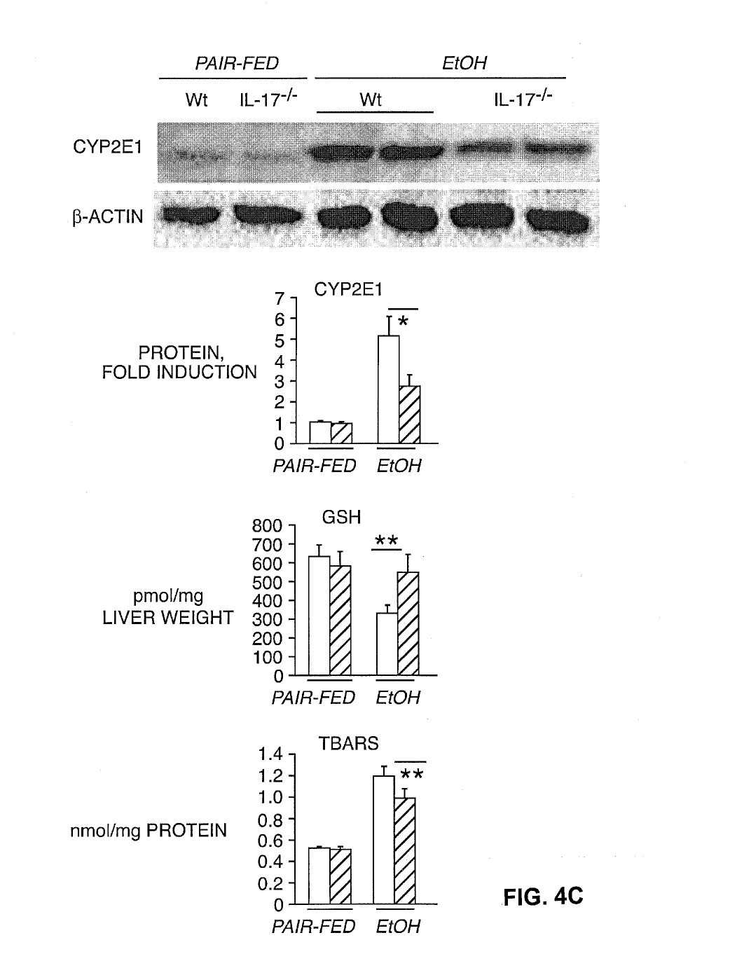

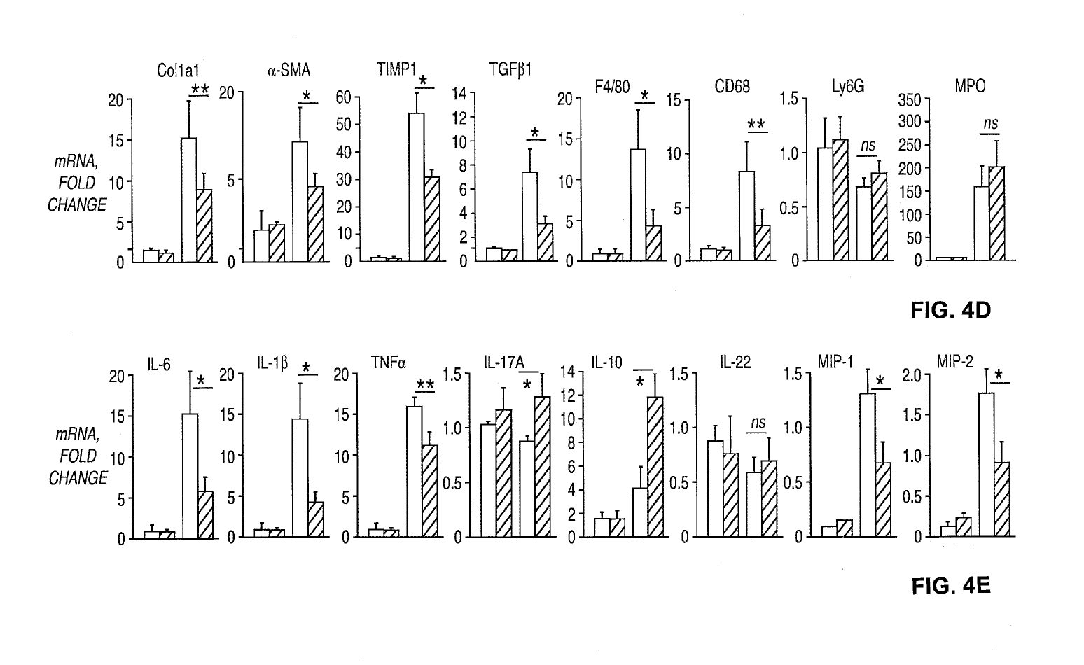

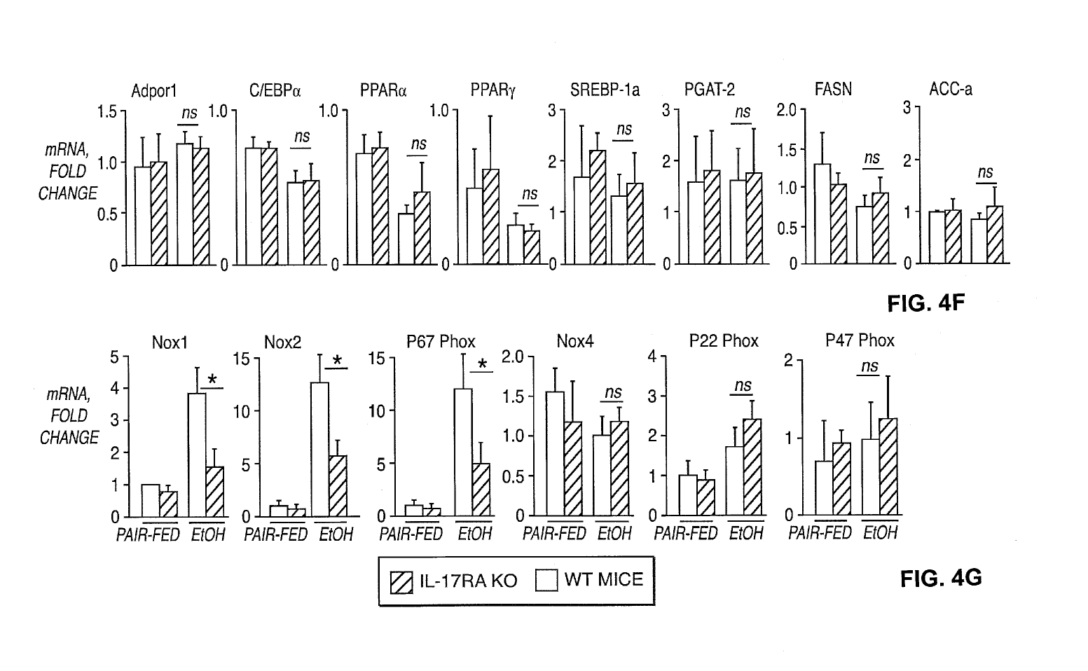

Liver fibrosis was induced in IL-17RA^{-/-} mice (16 w old, n=4) compared to pair-fed wt littermates (C57BL/6, 16 w old, n=5). Livers were analyzed as described herein. Tissue analysis: A) Based on ALT and AST measurements, liver injury was decreased in alcohol-fed IL-17RA^{-/-} mice (FIG. 4A). B) IL-17RA^{-/-} exhibited less steatosis as shown by H&E, and reduced content of hepatic triglycerides (FIG. 4B). Surprisingly, we did not observed any differences in lipogenic genes Adipor1, C/EBPα, PPARα, PPARγ, CREBP-1, PGAT2, FASN and ACC-α mRNA expression (FIG. 4F), suggesting that the IL-17 signaling does not affect de novo lipidogenesis, but rather regulates lipid secretion and/or beta oxidation. C) Hepatic lipid peroxidation was significantly reduced in alcohol-fed IL-17RA^{-/-} mice (as indicated by reduced levels 4-HNE and TBARS and upregulation of GSH, FIGS. 4B-C). In concordance, inhibition of ROS generation (fold reduction ↓1.8 Nox1, ↓1.5 Nox2, ↓2,5 P67Phox) was observed in livers of alcohol-fed IL-17RA^{-/-} mice, while TGF-β-induced Nox4, P22Phox, P47Phox remained unchanged, and suggested that IL-17 might regulate production of phagocytic and non-phagocytic ROS (FIG. 4G). D) Development of liver fibrosis was significantly reduced in IL-17RA^{-/-} mice, as demonstrated by reduced area of Sirius Red staining (↓3 fold) and downregulation of Col1α1 (↓1.5 fold), α-SMA, TIMP1, and TGFβRI mRNA expression (FIG. 4D). We detected significantly fewer Desmin⁺α-SMA⁺ HSCs (↓2.7 fold) in livers of alcohol-fed IL-17RA^{-/-} mice, compared to wt mice (FIG. 4B). Our data suggest that IL-17 plays a critical role in the pathogenesis of alcoholic fibrosis, and IL-17 may directly regulate HSC activation. E) Inflammatory response to alcohol was strongly inhibited in IL-17RA^{-/-} mice. Reduced expression of proinflammatory cytokines (fold ↓3 IL-6, ↓2.7 IL-1β, ↓1.5 TNF-α, ↓2 MIP1 and ↓2 MIP2, ↓2.7 F4/80 ↓1.4 CD68 of mRNA) in livers of IL-17RA^{-/-} mice correlated with low numbers of F4/80⁺ and CD68⁺ cells compared to wt mice (FIGS. 4B-E). Meanwhile, expression of MPO and Ly6G mRNA was not changed among alcohol-fed wt and IL-17RA−/− mice, suggesting that Kupffer cells/macrophages (but not neutrophils) mediate progression from steatohepatitis to fibrosis at this stage of ALD. Consistently, the mRNA levels of TGF-β1 were reduced (↓2.6 fold) in livers of IL-17RA^{-/-} mice, while IL-10 mRNA was strongly upregulated (↑3 fold, FIG. 4E). Our data suggest that IL-17 might regulate ALD by stimulating TGF-β1-producing and inflammatory (M1) Kupffer cells, and suppressing IL-10-producing macrophages (M2). Despite the significance of these results, this experiment must be confirmed with a larger number of mice.

B. Experimental Design:

i) the Role of IL-17 in Alcohol-Induced Fibrosis.

IL-17RA^{-/-}, IL-17^{-/-} and wt mice (males, 16 w old, see “Vertebrate animals”) are subjected to intragastric alcohol feeding (8 weeks), and the liver fibrosis compared to sham controls. While not intending to limit the invention to any particular mechanism, we hypothesize that IL-17RA^{-/-} and IL-17^{-/-} mice develop less fibrosis than the pair-fed wt mice due to reduced activation of Kupffer cells and HSCs/myofibroblasts. Liver tissues are analyzed as described (in 1.3). Tissue analysis: A) Liver fibrosis is measured in these mice by 1) Hydroxyproline assay; 2) Sirius Red staining; 3) staining for markers of activated myofibroblasts, α-SMA and desmin; and 4) qRT-PCR for expression of the fibrogenic genes α-SMA, Collagen-α1(I), Collagen-α2(I), TGF-βRI, TIMP1, MMP13, and MMP2^{58,80}. Hepatic and serum levels of inflammatory cytokines (TGF-β1, IL-6, IL-1β, IL-18, IL-17, IL-22, IL-23, IL-25, IL-27, TNFα, IFNγ, and CCL2) are measured by qRT-PCR and ELISA⁵⁸. Isolated Kupffer cells are analyzed for expression of TGF-β1, M1 (IL-β, TNF-α,IFN-γ) and M2 (Arg1, Mg11, Mrc2, IL-10) markers by qRT-PCR and flow cytometry^{58,105}. B) To further explore IL-17 effects on alcoholic fibrosis, livers from alcohol-fed IL-17RA^{-/-} and wt mice are analyzed using the Whole mouse genome microarray (Affymetrix Hgu133plus), and the gene expression profile of alcohol-injured mouse livers is indirectly compared to that in humans (reported by Affo et al. using the same GeneChips⁴⁸). In particular, specific upregulation of Fas, TNFRSF12A (Fn14), TNFα, TNFRSF1, TRAF1, TRAF3, TRAILR1 and TNFSF12 (TWEAK) mRNA distinguishes alcohol-induced cirrhosis from liver injury of other etiologies⁴⁸. Furthermore, induction of chemokine mRNAs (CCL2, CXCL1, CCL5, CXCL3, CXCL4, CXCL5, CXCL6, CXCL10)⁴⁸ in IL-17RA^{-/-} mice are compared to pair-fed wt mice (versus sham uninjured mice). C) Activation of CYP2E1 and intracellular signaling (AKT, p38 MAP kinase)³ critical for alcohol-induced liver fibrosis are evaluated in these mice^{106,107}. D) Hepatic lipid peroxidation and ROS production are compared in IL-17RA^{-/-} mice and pair-fed wt mice. We specifically check if phagocytic Nox2 and non-phagocytic Nox1^{108,109} are downregulated in livers of IL-17RA^{-/-} mice. E) Our preliminary data suggested that ablation of IL-17 inhibits hepatic steatosis (FIG. 4B). We will determine the cause of reduced lipidogenesis: a defect in 1) de novo lipid synthesis, 2) in fatty β-oxidation, or 3) lipid secretion in VLDL (see methods). Expression of adipogenic genes (PPARγ, PPARα, CEBP1, Adipor, Adpf1, SREBP-1, PGAT-1, PGAT-2, FASN, ACC-a) is assessed in isolated wt and IL-17RA-deficient hepatocytes and HSCs by qRT-PCR. Activation of HSCs during fibrogenesis is associated with downregulation of lipogenic genes^{80,110,111}. While deletion of IL-17RA does not change de novo adipogenesis in hepatocytes (FIG. 4F), it may affect expression of lipogenic genes and activation of HSCs. F) Secretion of hepatic and serum cytokines is measured by ELISA⁵⁸.

Activation/phosphorylation status of major signaling pathways utilized by the above mentioned cytokines, including STAT3, STAT1, NFκB (p65), IKKβ1 and JNK, is evaluated by Western blot analysis⁴⁸. The correlation between the global hepatic gene expression in pair-fed wt, IL-17RA^{-/-}, IL-17^{-/-} mice is established and compared to the data obtained from ALD patients¹¹².

ii) Identification of IL-17 Producing Cells and IL-17 Target Cells.

A) Flow cytometry is performed (similar to that described in AIM1 1.3) to identify the cellular source(s) of IL-17 in wt mice. In one embodiment, ALD progression to liver fibrosis is associated with increased IL-17 production by CD4⁺ T cells, and possibly, by other hematopoietic cells⁴³. B) IL-17-target cells are identified by upregulation of IL-17RA expression in response to intragastric alcohol feeding. In one embodiment, at this stage of ALD, besides CD4 T cells, Kupffer cells and HSCs are the major targets of IL-17 signaling.

iii) the Role of IL-17 Signaling in Kupffer Cells, Hepatocytes and HSCs in Alcoholic Fibrosis.

While not intending to limit the invention to any particular mechanism, we hypothesize that IL-17 signaling in Kupffer cells and HSCs (but not in hepatocytes) is critical for the pathogenesis of alcoholic liver fibrosis.

### Kupffer Cells.

Kupffer cell-specific IL-17RA-knockout KC^{IL-17RA-/-} mice and wt KC^{IL-17RA-wt/wt} mice are subjected to alcoholic liver fibrosis and analyzed as described herein. In one embodiment, secretion of TGF-β1 is reduced in alcohol-fed KC^{IL-17RA-/-} mice, and as a consequence, activation of aHSCs is inhibited. Furthermore, lack of IL-17 signaling in Kupffer cells can result in reduced expression of IL-6 and IL-1β, leading to reduced hepatic inflammation and attenuation of alcoholic fibrosis in KC^{IL-17RA-/-} mice. To test this hypothesis, IL-6- and IL-1β-induced activation of STAT3 and NFκB signaling pathways will be dissected in IL-17RA^{-/-} and wt Kupffer cells. In addition, the global cytokine secretion is compared in KC^{IL-17RA-/-} and wt KC^{IL-17RA-wt/wt} mice. We test if IL-17RA deletion in Kupffer cells causes compensatory upregulation of IL-17 (similar to that observed in IL-17RA^{-/-} ko mice)⁸⁷.

### Hepatic Stellate Cells.

Alcoholic liver fibrosis is induced in HSC^{IL-17RA-/-} mice and HSC^{IL-17RA-wt/wt} mice. In one embodiment, IL-17-deficient aHSCs exhibit a defect in activation and collagen type I production, and therefore, HSC^{IL-17RA-/-} mice are protected from liver fibrosis. IL-17 was shown to activate STAT3 signaling in macrophages³⁹ and aHSC/myofibroblasts⁵⁸. To determine the mechanism of IL-17 signaling in HSCs, activation of NFκB and STAT3 signaling pathways are compared in IL-17RA^{-/-} and wt aHSCs. In one embodiment, IL-17 binding to IL-17RA can directly activate STAT3 signaling in HSCs⁵⁸ and via induction of IL-6, a STAT3-signaling cytokine⁵⁸. Furthermore, we will test if blocking STAT3 (using STAT3-specific inhibitor S31-201 (nifuroxazide), 5 mg/kg, i.p., every 2nd day, Millipore)¹¹³⁻¹¹⁵ can prevent HSC activation.

### Hepatocytes.

We will test if Hep^{IL-17RA-/-} mice develop less steatosis and fibrosis. Based on our preliminary data, Hep^{IL-17RA-/-} mice develop less steatosis (FIG. 4B). Previous studies have established that steatosis alone is not sufficient to induce alcoholic liver fibrosis, but requires “a second hit”^{13,48,116.} Here we examine if deletion of IL-17RA in hepatocytes affects development of liver fibrosis. For this purpose, livers from alcohol-fed Hep^{IL-17RA-/-} mice and Hep^{IL-17RA-wt/wt} mice are compared.

C. Potential Outcome:

1) In one embodiment, IL-17RA^{-/-} and IL-17^{-/-} mice are protected from alcohol-induced fibrosis. In one embodiment, IL-17 signaling in Kupffer cells and aHSCs plays a critical role in the pathogenesis of alcoholic fibrosis.
2) As an alternative approach, alcoholic liver fibrosis is induced in IL-23p19^{-/-}, IL-25^{-/-} and IL-27p28^{-/-} and wt mice. In one embodiment, liver fibrosis is attenuated in IL-23^{-/-} mice.

D. Methods:

### Chronic Intragastric Ethanol Feeding⁸⁰:

C57BL/6 male (16 wks old) mice are first fed ad libitum “Western diet”, ±a solid diet high in cholesterol and saturated fat (HCFD:1% w/w cholesterol, 21% calories from lard, 17% calories from corn oil) for 2 weeks. The mice are then operated for implantation of gastric catheters for intragastric feeding of liquid high fat diet (36% calories from corn oil) plus ethanol or isocaloric dextrose at 60% of daily caloric intake for 8 weeks for males^{101,102}. Ethanol dose is increased from 19 to 32 g/kg/day. During 8 w intragastric feeding period, mice continue to consume ad libitum HCFD to match caloric intake.

### Assessment of In Vivo Lipogenesis:

To measure hepatic de novo lipogenesis in vivo. 5 μCi [¹⁴C] palmitate will be injected into mice, and livers will be harvested 2 minutes following the injection¹¹⁷. For triglyceride synthesis assays, lipid will be extracted by Folch method, and then separated by thin layer chromatography. Assessment of in vivo fatty acid β-oxidation: To measure hepatic FAO in vivo. 5 μCi [¹⁴C] palmitate is injected into alcohol-fed IL-17RA^{-/-} and wt mice. Livers are harvested 2 min after the injection¹¹⁷. FAO acid-soluble metabolites and captured CO₂ are measured by scintillation counter.

### Measurement of VLDL TG Secretion Rates:

Mice are fasted for 4 h and intraperitoneally injected with 400 μL P-407 (1 mg/g) (Calbiochem) solution in PBS¹¹⁸. Blood is collected from tail vein prior to injection (0 h) and at 1, 2, 4 and 8 h after injection. TG are measured and VLDL TG secretion rate is calculated as the slope of the plasma TG concentration vs time after linear regression, and expressed in mg/dL/h.

### Example 18

### Characterizing the Role of IL-17 in Alcohol-Induced HCC

Meanwhile, little is known about the effects of ethanol on HCC progression. Previous studies have demonstrated that addition of ethanol to drinking water increased tumor incidence in DEN-injected male mice⁸². Here we use DEN+alcohol drinking to investigate IL-17 in the development of HCC in IL-17RA^{-/-} mice compared to wt mice.

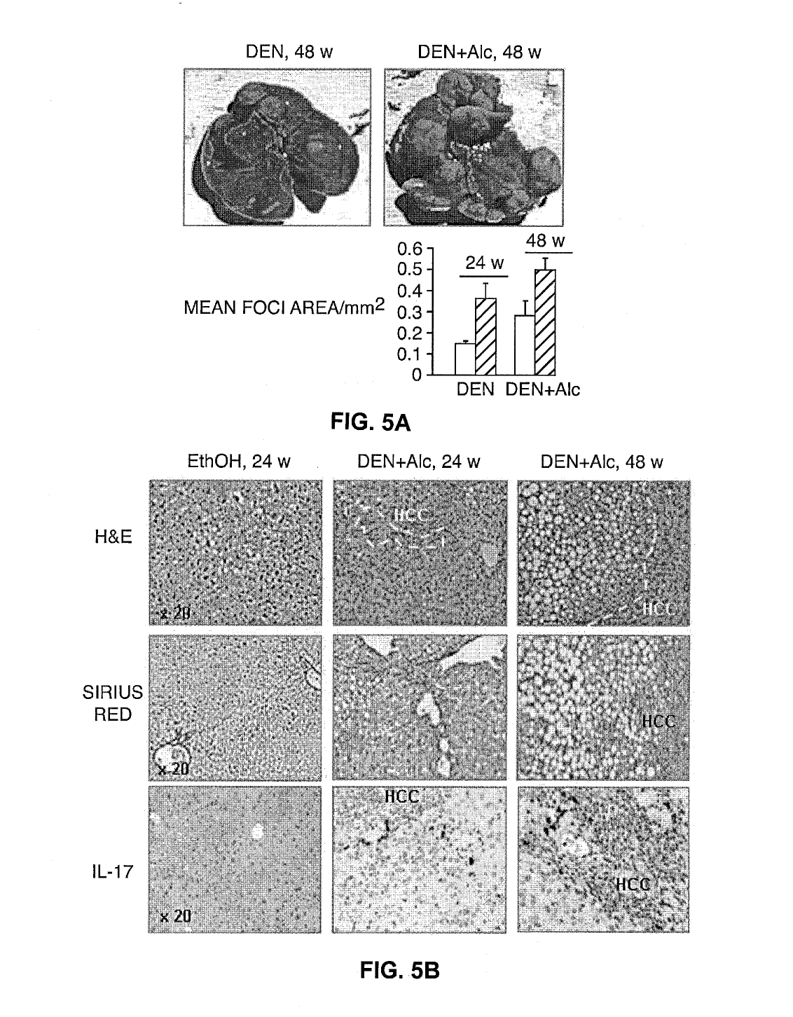

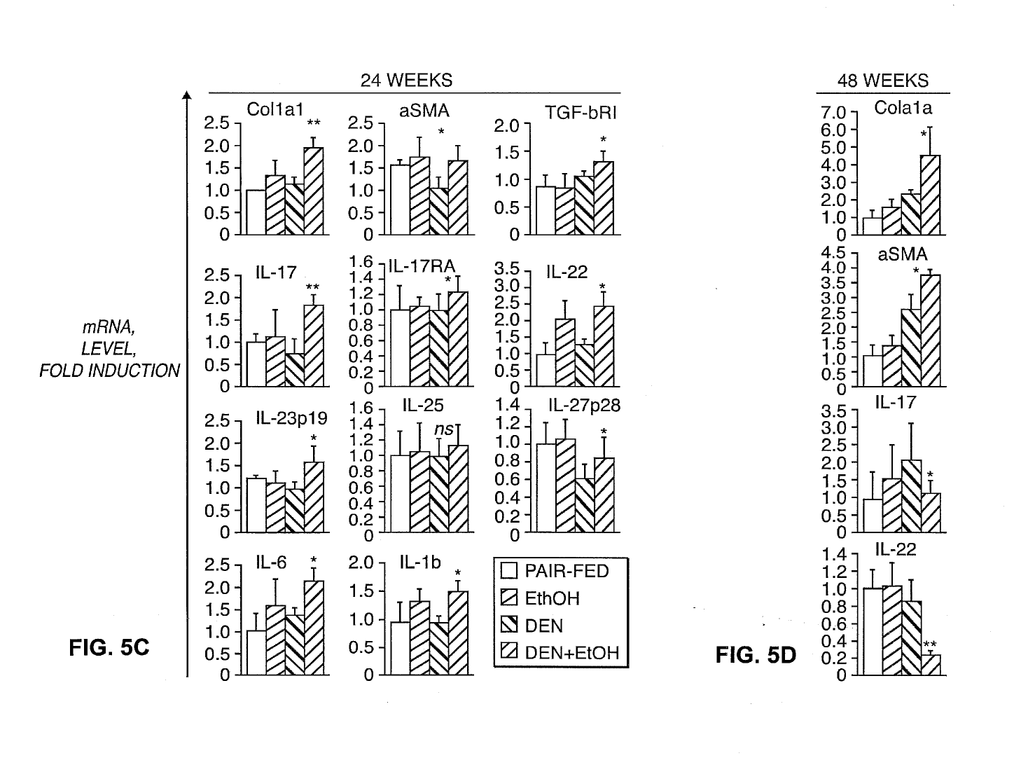

Alcohol consumption facilitates development of HCC in patients with liver fibrosis^{1,48}. However, previous studies of HCC have been limited by the lack of appropriate mouse models. It has been recently demonstrated that DEN-alcohol-induced HCC in mice histologically resemble human HCC, and that chronic ethanol feeding accelerates liver fibrosis and HCC^{82,119,120}. Based on our preliminary data, development of HCC in DEN-alcohol fed mice is associated with high levels of IL-6 and IL-1β¹²¹⁻¹²³, IL-17, IL-17RA, and IL-23 (FIG. 5). Furthermore, enrichment of Th17 cells was detected in the tumors of HCC patients^{124,125}, suggesting that IL-17 may serve as a new therapeutic target^{124,125}. We hypothesize that ablation of IL-17 signaling in mice will attenuate DEN-alcohol-induced tumorigenesis via 1) inhibition of liver fibrosis due to reduced secretion of TGFβ1 by Kupffer cells and HSC activation; and 2) inhibition of tumorigenesis due to reduced production of IL-6 production by Kupffer cells and other STAT3-signaling cytokines.

A. Results:

Male mice (3 weeks old) were injected with a single dose of DEN (i.p. 1 μg/g body weight in corn oil) or vehicle¹²⁶, and at 16 weeks of age, or 40 weeks of age were subjected to alcohol feeding (for 8 weeks) for early and late carcinogenesis study (see methods). Upon gross examination, visible lesions were observed in both DEN- and DEN-alcohol-treated mice, with a significantly greater number being visible in DEN-alcohol-treated mice (FIG. 5A). These findings were confirmed by quantifying foci area in H&E-stained livers of DEN- and DEN-alcohol-treated mice in groups at 24 weeks of age, and 48 weeks of age.

Tumorigenesis in DEN-alcohol-treated mice (vs pair-fed mice) was complicated by progressive development of steatosis and liver fibrosis (FIG. 5B) and correlated with increased expression of Col1α1, αSMA, and TGF-βRI mRNA (FIG. 5C). IL-17-expressing cells were detected in livers of DEN-alcohol mice, and were scattered close to HCC. We detected mild elevation of hepatic IL-6 and IL-1β expression in DEN-alcohol-treated mice at 24 weeks of age. Moreover, IL-17, IL-17RA, IL-22 and IL-23 expression was ≈1.5 times induced compared with age-matched DEN-treated mice. Further evaluation revealed that at 48 weeks of age, expression of inflammatory cytokines is suppressed, suggesting that long-term alcohol feeding induces severe immunosuppressive effect in DEN-alcohol-treated mice, causing increased tumor incidence (vs pair-fed mice). These results suggest that alcohol-induced microenvironment drives HCC progression.

B. Experimental Design:

i) Chronic Alcohol Feeding Complicates DEN-Induced HCC in Wt Mice.

First, we will optimize the DEN-alcohol HCC model in wt mice to achieve tumor incidence <70-80% in wt mice^{82,119}. For this purpose, wt mice are injected with DEN (i.p. 1 μg/g body weight in corn oil) at 3 weeks of age (rather than at 2 weeks of age, which results in tumor incidence in 100% of mice without EtOH feeding)^{127,128}. These mice are subjected to early (at 24 weeks of age) and late (at 48 weeks of age)⁸² alcohol feeding for 8 weeks⁸⁰. The tumor incidence, multiplicity, size, load¹²⁹ and liver fibrosis are determined in alcohol-, DEN-, and DEN-alcohol-treated and pair-fed wt MiCe^{129,130}. Tissue analysis. A) HCC nodules are identified in these mice by H&E staining, and expression of α-fetoprotein (AFP), glypican 3 (Gpc3), and Ly6D, CD90, CD133 and EpCAM²⁰. Hepatic fibrosis is evaluated by staining with Sirius Red, anti-αSMA and anti-Desmin Abs, qRT-PCR for Col1α1, αSMA, TIMP1 and TGFβRI mRNA expression. Hepatic inflammation is determined by the level of mRNA expression of cytokines and inflammatory cell markers. Based on our preliminary data, elevated levels of TGF-β1, IL-6 and IL-17 cause activation of HSCs, and development of liver fibrosis that facilitates development of HCC. B) Hepatic steatosis is estimated by H&E, detection of tryglicerides, and expression of lipogenic genes. Hepatic levels of oxidative stress (TBARS, induction of 4-NHE and depletion of GSH levels) is measured¹². C) Large HCC nodules are surgically separated from ‘non-tumor’ tissue using dissecting light microscope¹²⁹, and expression of tumor-associated and proliferation markers is compared in tumor (HCC nodules) and non-tumor tissues¹²⁹. HCC progression is associated with abnormal proliferation of hepatocytes. Expression of cyclin D1, Bcl2, BcxL and activation of STAT3 is evaluated in HCC nodules and non-tumors by qRT-PCR and Western blotting. D). Transformed hepatocytes undergo apoptosis within HCC nodule (detected by TUNEL assay, caspase 3 expression), causing activation of Kupffer cells/macrophages and release of IL-6, a strong tumorigenic cytokine induces constitutive STAT3 activation in transformed hepatocytes. Since IL-6, IL-17 and IL-22 are implicated in promoting HCC via STAT3 activation, the levels of IL-6, IL-17, IL-22, phosphorylation of STAT3 are measured by qRT-PCR, ELISA and Western blotting. E) Non-tumor tissue provides microenvironment for tumor progression. Chronic alcohol injury may (versus DEN alone) may affect the non-tumor tissue to secrete factors to promote HCC growth. Therefore, non-tumor tissues from DEN- and DEN-alcohol-treated wt mice are compared. We evaluate the levels of IL-17A, IL-17RA, IL-22, IL-23, CCL2, TGF-β1, PDGF, CTGF mRNA expression in the livers of DEN-alcohol-treated wt mice versus DEN-treated wt mice.

ii) Role of IL-17 Signaling in DEN-Alcohol-Induced HCC.

Development of HCC is compared in age matched (males, 24 weeks of age) DEN-alcohol-treated IL-17RA^{-/-}, IL-17^{-/-}, and wt mice (n=10/group; versus pair-fed mice). While not intending to limit the invention to any particular mechanism, we hypothesize that HCC development is attenuated in DEN-alcohol-treated IL-17RA^{-/-} and IL-17^{-/-} mice compared to wt mice.

Tissue analysis is performed as described herein A) In one embodiment, tumor multiplicity, size and load¹²⁹ are reduced in DEN-alcohol-treated IL-17RA^{-/-} mice and are associated with lower level of collagen Type I deposition and inflammation compared to pair-fed wt littermates. B) In one embodiment, proliferation of HCC from DEN-alcohol-treated IL-17RA^{-/-} mice is reduced compared to pair-fed wt mice. In addition to D1, Bcl2, BcxL mRNA levels, we will measure the level of BrdU incorporation. For this purpose, DEN-alcohol-treated IL-17RA^{-/-} and wt mice (n=5/group) are BrdU pulsed 2 hr prior to sacrifice and livers are stained with anti-BrdU Ab (and with anti-Ki67 Ab). C) The role of IL-17 signaling in HCC progression is studied in HCC nodules and non-tumor tissue isolated from from DEN-alcohol-treated IL-17RA^{-/-} and wt mice. In one embodiment, IL-6 expression and STAT3 phosphorylation are reduced in HCC nodules from IL-17RA^{-/-} mice (vs wt mice) due to the lack of IL-17-driven feedback mechanism regulating IL-6-dependent HCC proliferation⁴³. Activation of the components of STAT3 signaling cascade is evaluated by Western blotting of the HCC nodules. Furthermore, a single HCC cell suspension is obtained from wt and IL-17RA^{-/-} HCC nodules (as described in¹²⁹). HCC-derived IL-17RA^{-/-} and wt cells are stimulated with IL-6 (IL-17 or IL-22), phosphorylation of gp130, Jak2¹³¹ and Jak1 as well as nuclear translocation of phospho-STAT3 are evaluated¹³². Expression of the tumorigenic STAT3 target genes is detected by RT-PCR. D) In one embodiment, the levels of TNF-α and IL-1β (and NF-κB, and IKKγ activation) are reduced in IL-17RA^{-/-} mice (vs wt mice) due to inhibition of inflammatory responses in DEN-alcohol-treated IL-17RA^{-/-} mice. We examine TNF-α-dependent signaling cascade: phosphorylation of IκB proteins by IKKα and IKKγ protein kinases which results in IκB ubiquitination and degradation, and release and nuclear translocation of phospho-NF-κB subunits that mediate proinflammatory responses¹³³. We also examine an alternative pathway triggered by TNFRI crosslinking: Phosphorylation of mitogen-activated protein kinases (MAPKs), TRAF3 degradation, release of MAPK3 from inhibitory TRAF2/6 complex and activation of NF-κB. Activation of this signaling cascade results in rapid delivery of NF-κB-dependent survival signals and conversion of JNK-promoted death signals into proliferative signals¹³⁴ in HCC-derived cells. E) The surrounding non-tumor tissues from IL-17RA^{-/-} and wt mice are analyzed as described herein. In one embodiment, tissue microenvironment surrounding HCC nodules in IL-17RA^{-/-} mice differs from that in wt mice.

iii) the Role of IL-17 Signaling in Kupffer Cells, Hepatocytes and HSCs in DEN-Alcohol-Induced HCC. Kupffer Cells.

Here we test if deletion of IL-17RA in Kupffer cells results in inhibition of DEN-alcohol induced HCC due to reduced hepatic inflammation (autocrine IL-6 production) and fibrosis (production of TGF-β1). For this purpose, development of HCC is compared in DEN-alcohol KC^{IL-17RA-/-} mice, wt KC^{IL-17RA-wt/wt} mice and KC^{IL-6-/-} mice (generated by crossing of IL-6^{flox/flow} mice¹³⁵ with Lysozyme-Cre mice). The responses of Kupffer cells from these mice to IL-6 and TNFα are compared in vivo and in vitro as described herein. In one embodiment, IL-6 production is reduced in IL-17RA^{-/-} Kupffer cells to the level comparable to that in IL-6^{-/-} Kupffer cells, suggesting that IL-17/IL-6 autocrine loop plays an important role in Kupffer cell activation. In one embodiment, DEN-alcohol KC^{IL-17RA-/-} mice develop less tumors than their wild type littermates (comparable to that in KC^{IL-6-/-} mice formation) and is associated with the reduced activation of STAT3 in HCC hepatocytes. In addition, in one embodiment, secretion of TGF-β1 is reduced in KC^{IL-17RA-/-} mice, and as a consequence, activation of aHSCs is inhibited.

### Hepatic Stellate Cells.

We will test if alcohol-mediated activation of HSCs and subsequent collagen deposition provides a microenvironment that facilitates HCC development in DEN-alcohol-treated mice. Therefore, abrogation of activation signals in DEN-alcohol-treated HSC^{IL-17RA-/-} mice will result in inhibition of HCC formation compared to HSC^{IL-17RA-wt/wt} mice.

### Hepatocytes.

Hepatocyte-specific Hep^{IL-17RA-/-} mice and Hep^{IL-17RA-wt/wt} mice are generated. In one embodiment, HCC formation is inhibited in Hep^{IL-17-/-} mice due to reduced IL-17/IL-6/STAT3 activation in hepatocytes.

C. Potential Outcome:

1) IL-17 is believed to facilitate development of HCC and metastatic spread^{4,26}. In one embodiment, tumor incidence is decreased in DEN-alcohol-treated IL-17RA^{-/-} mice (vs wt mice). In one embodiment, IL-17-signaling in all three cell types, Kupffer cells, HSCs and hepatocytes, contributes to the pathogenesis of DEN-alcohol-induced HCC.
2) As an alternative approach, the role of IL-23, IL-25 and IL-27 in the pathogenesis of HCC can be evaluated. IL-23p19^{-/-}, IL-25^{-/-} and IL-27p28^{-/-} and wt mice are subjected to DEN-alcohol injury, and development of liver fibrosis and tumor formation are analyzed in these mice.

D. Methods:

### DEN+Alcohol HCC Model.

Male mice (3 weeks old) are injected with a single dose of diethylnitrosamine (DEN, i.p. 1 μg/g body weight in corn oil) or vehicle¹²⁶. Following DEN injury, mice are subjected to alcohol feeding model at 16 weeks of age or 40 weeks of age for early and late carcinogenesis study. Animals assigned to ethanol-feeding are weaned onto an ethanol drinking water (Alcohol) regime by replacing water with 5% (v/v) EtOH in water (3 days), followed by 10% (v/v) EtOH in water (3 days). At the end of this period, animals are maintained on 10/20% (v/v) alcohol (alternate days) for a further 8 weeks. For animals assigned to the early hepatocarcinogenesis studies, initiation of ethanol feeding begins at 16 weeks and ceases at 24 weeks of age. For animals assigned to the late hepatocarcinogenesis studies, initiation of ethanol feeding begins at 40 weeks and ceases at 48 weeks of age. Mice are sacrificed (at 24 weeks, or 48 weeks of age) within 1 h after termination of alcohol feeding.

### Example 19

### Preclinical Studies—Pharmacological Treatment of ALD with IL-17 Antagonists

Previous experiments provide a strong genetic justification for testing IL-17 inhibitors in mouse models of ALD. In one embodiment, the intragastric alcohol feeding model is used for preclinical studies. A). C57BL6 mice will be subjected to intragastric alcohol feeding (8 weeks), and the liver fibrosis compared to age-matched pair-fed controls. Alcohol-fed mice are treated weekly by intraperitoneal injection with mouse anti-IL-17A antibody (500 μg Ab/mouse, Clone MM17F3, Bio X Cell) or isotype control antibody. The anti-IL-17 Ab is administered immediately after alcohol feeding (prophylactic dosing), or with one month delay (therapeutic dosing). This Ab was successfully used for prevention of experimental autoimmune encephalomyelitis (EAE) in mice¹³⁶. (Successful use of other anti-IL-17 antibodies has been reported: Two monoclonal anti-IL-17A antibodies ixekizumab (LY2439821) from Eli Lilly, and secukinumab (also known as AIN457, described in EP 1776142 B1, published in W02006013107A1) (Novartis, U.S.A.)) have been tested in clinical trials, while anti-IL-17A Abs SCH-900117 and RG4934 are in early clinical development)⁴⁰. B). In addition to neutralizing Abs, small molecules have been developed to target IL-17 pathway. These include halofuginone (HF)^{137,138,} Ursolic acid (UA)¹³⁹, and SR1001¹⁴⁰. In addition to IL-17 inhibition, HF^{137,138} possesses strong anti-fibrotic (by inhibiting Col1a1 and Col2a1 expression^{141,142}), and anti-tumorigenic properties¹⁴³, which makes HF an attractive drug to target ALD progression. The appropriate dosing of each compound will be determined. Based on the published studies, the following concentrations will be used: HF (0.25 mg/kg of body weight, i.p. twice a week); UA (150 mg/kg of body weight by i.p, every second day); SR1001 (25 mg/kg of body weight, i.p. twice a week)¹⁴⁰. The compounds will be administered to alcohol-fed (or pair-fed) mice immediately after development of liver injury or with one month delay.

### REFERENCES IN EXAMPLES 1-14

1. Bataller R, Brenner D A. Liver fibrosis. J Clin Invest 2005; 115:209-18.
2. Seki E, De Minicis S, Osterreicher C H, et al. TLR4 enhances TGF-beta signaling and hepatic fibrosis. Nat Med 2007; 13:1324-32.
3. Friedman S L, Roll F J, Boyles J, et al. Hepatic lipocytes: the principal collagen-producing cells of normal rat liver. Proc Natl Acad Sci USA 1985; 82:8681-5.
4. Steinman L. A brief history of T(H)17, the first major revision in the T(H)1/T(H)2 hypothesis of T cell-mediated tissue damage. Nat Med 2007; 13:139-45.
5. Kolls J K, Linden A. Interleukin-17 family members and inflammation. Immunity 2004; 21:467-76.
6. Iwakura Y, Ishigame H, Saijo S, et al. Functional specialization of interleukin-17 family members. Immunity 2011; 34:149-62.
7. Shen F, Gaffen S L. Structure-function relationships in the IL-17 receptor: implications for signal transduction and therapy. Cytokine 2008; 41:92-104.
8. Subramaniam S V, Cooper R S, Adunyah S E. Evidence for the involvement of JAK/STAT pathway in the signaling mechanism of interleukin-17. Biochem Biophys Res Commun 1999; 262:14-9.
9. Radaeva S, Sun R, Pan H N, et al. Interleukin 22 (IL-22) plays a protective role in T cell-mediated murine hepatitis: IL-22 is a survival factor for hepatocytes via STAT3 activation. Hepatology 2004; 39:1332-42.
10. Ge J, Wang K, Meng Q H, et al. Implication of Th17 and Th1 cells in patients with chronic active hepatitis B. J Clin Immuno12010; 30:60-7.
11. Lemmers A, Moreno C, Gustot T, et al. The interleukin-17 pathway is involved in human alcoholic liver disease. Hepatology 2009; 49:646-57.
12. Xu L, Hui A Y, Albanis E, et al. Human hepatic stellate cell lines, LX-1 and LX-2: new tools for analysis of hepatic fibrosis. Gut 2005; 54:142-51.
13. Schnabl B, Choi Y H, Olsen J C, et al. Immortal activated human hepatic stellate cells generated by ectopic telomerase expression. Lab Invest 2002; 82:323-33.
14. Yata Y, Scanga A, Gillan A, et al. DNase I-hypersensitive sites enhance alphal (I) collagen gene expression in hepatic stellate cells. Hepatology 2003; 37:267-76.
15. Ye P, Rodriguez F H, Kanaly S, et al. Requirement of interleukin 17 receptor signaling for lung CXC chemokine and granulocyte colony-stimulating factor expression, neutrophil recruitment, and host defense. J Exp Med 2001; 194:519-27.
16. Nakae S, Komiyama Y, Nambu A, et al. Antigen-specific T cell sensitization is impaired in IL-17-deficient mice, causing suppression of allergic cellular and humoral responses. Immunity 2002; 17:375-87.
17. Takeda K, Clausen B E, Kaisho T, et al. Enhanced Th1 activity and development of chronic enterocolitis in mice devoid of Stat3 in macrophages and neutrophils. Immunity 1999; 10:39-49.
18. Yanagida A, Iwaisako K, Hatano E, et al. Downregulation of the Wnt antagonist Dkk2 links the loss of Sept4 and myofibroblastic transformation of hepatic stellate cells. Biochim Biophys Acta 2011; 1812:1403-11.
19. Kisseleva T, Song L, Vorontchikhina M, et al. NF-kappaB regulation of endothelial cell function during LPS-induced toxemia and cancer. J Clin Invest 2006; 116:2955-63.
20. Zenewicz L A, Yancopoulos G D, Valenzuela D M, et al. Interleukin-22 but not interleukin-17 provides protection to hepatocytes during acute liver inflammation. Immunity 2007; 27:647-59.
21. Kang Z, Altuntas C Z, Gulen M F, et al. Astrocyte-restricted ablation of interleukin-17-induced Act1-mediated signaling ameliorates autoimmune encephalomyelitis. Immunity 2010; 32:414-25
22. Linden A. A role for the cytoplasmic adaptor protein Act1 in mediating IL-17 signaling. Sci STKE 2007; 2007:re4.
23. Oakley F, Meso M, Iredale J P, et al Inhibition of inhibitor of kappaB kinases stimulates hepatic stellate cell apoptosis and accelerated recovery from rat liver fibrosis. Gastroenterology 2005; 128:108-20.
24. Wang H, Lafdil F, Kong X, et al. Signal transducer and activator of transcription 3 in liver diseases: a novel therapeutic target. Int J Biol Sci 2011; 7:536-50.
25. Gaffen S L. Structure and signalling in the IL-17 receptor family. Nat Rev Immunol 2009; 9:556-67.
26. Maitra A, Shen F, Hanel W, et al. Distinct functional motifs within the IL-17 receptor regulate signal transduction and target gene expression. Proc Natl Acad Sci USA 2007; 104:7506-11.
27. Wilson M S, Madala S K, Ramalingam T R, et al. Bleomycin and IL-lbeta-mediated pulmonary fibrosis is IL-17A dependent. J Exp Med 2010; 207:535-52.
28. Faust S M, Lu G, Marini B L, et al. Role of T cell TGFbeta signaling and IL-17 in allograft acceptance and fibrosis associated with chronic rejection. J Immunol 2009; 183:7297-306.
29. Mair M, Zollner G, Schneller D, et al. Signal transducer and activator of transcription 3 protects from liver injury and fibrosis in a mouse model of sclerosing cholangitis. Gastroenterology; 138:2499-508.
30. Ogata H, Chinen T, Yoshida T, et al. Loss of SOCS3 in the liver promotes fibrosis by enhancing STAT3-mediated TGF-beta1 production. Oncogene 2006; 25:2520-30.

### REFERENCES IN “DEFINITIONS” AND IN EXAMPLES 15-18

1. Gao, B. & Bataller, R. Alcoholic liver disease: pathogenesis and new therapeutic targets. Gastroenterology 141, 1572-1585 (2011).
2. Diehl, A. M. Alcoholic liver disease: natural history. Liver transplantation and surgery: official publication of the American Association for the Study of Liver Diseases and the International Liver Transplantation Society 3, 206-211 (1997).
3. Mello, T., Ceni, E., Surrenti, C. & Galli, A. Alcohol induced hepatic fibrosis: role of acetaldehyde. Molecular aspects of medicine 29, 17-21 (2008).
4. Miller, A. M., Horiguchi, N., Jeong, W. I., Radaeva, S. & Gao, B. Molecular mechanisms of alcoholic liver disease: innate immunity and cytokines. Alcohol Clin Exp Res 35, 787-793 (2011).
5. O'Shea, R. S., Dasarathy, S., McCullough, A. J., Practice Guideline Committee of the

American Association for the Study of Liver, D. & Practice Parameters Committee of the American College of, G. Alcoholic liver disease. Hepatology 51, 307-328 (2010).
6. Lucey, M. R., Mathurin, P. & Morgan, T. R. Alcoholic hepatitis. The New England journal of medicine 360, 2758-2769 (2009).
7. Maltby, J., Wright, S., Bird, G. & Sheron, N. Chemokine levels in human liver homogenates: associations between GRO alpha and histopathological evidence of alcoholic hepatitis. Hepatology 24, 1156-1160 (1996).
8. Dominguez, M., et al. Hepatic expression of CXC chemokines predicts portal hypertension and survival in patients with alcoholic hepatitis. Gastroenterology 136, 1639-1650 (2009).
9. Lemmers, A., et al. The interleukin-17 pathway is involved in human alcoholic liver disease. Hepatology 49, 646-657 (2009).
10. Jones, C. E. & Chan, K. Interleukin-17 stimulates the expression of interleukin-8, growth-related oncogene-alpha, and granulocyte-colony-stimulating factor by human airway epithelial cells. American journal of respiratory cell and molecular biology 26, 748-753 (2002).
11. Dhanda, A. D., Lee, R. W., Collins, P. L. & McCune, C. A. Molecular targets in the treatment of alcoholic hepatitis. World J Gastroenterol 18, 5504-5513 (2012).
12. Ki, S. H., et al. Interleukin-22 treatment ameliorates alcoholic liver injury in a murine model of chronic-binge ethanol feeding: role of signal transducer and activator of transcription 3. Hepatology 52, 1291-1300 (2010).
13. Bataller, R. & Brenner, D. A. Liver fibrosis. J Clin Invest 115, 209-218 (2005).
14. Friedman, S. L. Mechanisms of hepatic fibrogenesis. Gastroenterology 134, 1655-1669 (2008).
15. Seki, E., et al. TLR4 enhances TGF-beta signaling and hepatic fibrosis. Nat Med 13, 1324-1332 (2007).
16. Friedman, S. L., Roll, F. J., Boyles, J. & Bissell, D. M. Hepatic lipocytes: the principal collagen-producing cells of normal rat liver. Proc Natl Acad Sci USA 82, 8681-8685 (1985).
17. Sell, S. Is there a liver stem cell? Cancer Res 50, 3811-3815 (1990).
18. Alison, M. R. Liver stem cells: implications for hepatocarcinogenesis. Stem cell reviews 1, 253-260 (2005).
19. Wu, X. Z. & Chen, D. Origin of hepatocellular carcinoma: role of stem cells. Journal of gastroenterology and hepatology 21, 1093-1098 (2006).
20. Shen, Y. & Cao, D. Hepatocellular carcinoma stem cells: origins and roles in hepatocarcinogenesis and disease progression. Frontiers in bioscience 4, 1157-1169 (2012).
21. Duncan, A. W., Dorrell, C. & Grompe, M. Stem cells and liver regeneration. Gastroenterology 137, 466-481 (2009).
22. Sell, S. Heterogeneity and plasticity of hepatocyte lineage cells. Hepatology 33, 738-750 (2001).
23. Naugler, W. E., et al. Gender disparity in liver cancer due to sex differences in MyD88-dependent IL-6 production. Science 317, 121-124 (2007).
24. Gu, F. M., et al. IL-17 induces AKT-dependent IL-6/JAK2/STAT3 activation and tumor progression in hepatocellular carcinoma. Molecular cancer 10, 150 (2011).
25. Li, J., et al. Interleukin 17A promotes hepatocellular carcinoma metastasis via NF-kB induced matrix metalloproteinases 2 and 9 expression. PLoS One 6, e21816 (2011).
26. Jiang, R., et al. Interleukin-22 promotes human hepatocellular carcinoma by activation of STAT3. Hepatology 54, 900-909 (2011).
27. Wang, H., Lafdil, F., Kong, X. & Gao, B. Signal transducer and activator of transcription 3 in liver diseases: a novel therapeutic target. Int J Biol Sci 7, 536-550 (2011).
28. Yu, H., Pardoll, D. & Jove, R. STATs in cancer inflammation and immunity: a leading role for STAT3. Nature reviews. Cancer 9, 798-809 (2009).
29. Sicklick, J. K., et al. Dysregulation of the Hedgehog pathway in human hepatocarcinogenesis. Carcinogenesis 27, 748-757 (2006).
30. Karin, M. NF-kappaB as a critical link between inflammation and cancer. Cold Spring Harbor perspectives in biology 1, a000141 (2009).
31. Sakurai, T., et al. Hepatocyte necrosis induced by oxidative stress and IL-1 alpha release mediate carcinogen-induced compensatory proliferation and liver tumorigenesis. Cancer Cell 14, 156-165 (2008).
32. White, B. D., Chien, A. J. & Dawson, D. W. Dysregulation of Wnt/beta-catenin signaling in gastrointestinal cancers. Gastroenterology 142, 219-232 (2012).
33. Kolls, J. K. & Linden, A. Interleukin-17 family members and inflammation. Immunity 21, 467-476 (2004).
34. Ivanov, I I, et al. The orphan nuclear receptor RORgammat directs the differentiation program of proinflammatory IL-17+T helper cells. Cell 126, 1121-1133 (2006).
35. Yang, X. O., et al. T helper 17 lineage differentiation is programmed by orphan nuclear receptors ROR alpha and ROR gamma. Immunity 28, 29-39 (2008).
36. Iwakura, Y., Ishigame, H., Saijo, S. & Nakae, S. Functional specialization of interleukin-17 family members. Immunity 34, 149-162 (2011).
37. Yao, Z., et al. Herpesvirus Saimiri encodes a new cytokine, IL-17, which binds to a novel cytokine receptor. Immunity 3, 811-821 (1995).
38. Andoh, A., et al. IL-17 selectively down-regulates TNF-alpha-induced RANTES gene expression in human colonic subepithelial myofibroblasts. J Immunol 169, 1683-1687 (2002).
39. Subramaniam, S. V., Cooper, R. S. & Adunyah, S. E. Evidence for the involvement of JAK/STAT pathway in the signaling mechanism of interleukin-17. Biochem Biophys Res Commun 262, 14-19 (1999).
40. Miossec, P. & Kolls, J. K. Targeting IL-17 and TH17 cells in chronic inflammation. Nature reviews. Drug discovery 11, 763-776 (2012).
41. Ye, P., et al. Requirement of interleukin 17 receptor signaling for lung CXC chemokine and granulocyte colony-stimulating factor expression, neutrophil recruitment, and host defense. J Exp Med 194, 519-527 (2001).
42. Shen, F. & Gaffen, S. L. Structure-function relationships in the IL-17 receptor: implications for signal transduction and therapy. Cytokine 41, 92-104 (2008).
43. Gaffen, S. L. Structure and signalling in the IL-17 receptor family. Nat Rev Immunol 9, 556-567 (2009).
44. Faust, S. M., et al. Role of T cell TGFbeta signaling and IL-17 in allograft acceptance and fibrosis associated with chronic rejection. J Immunol 183, 7297-7306 (2009).
45. Wilson, M. S., et al. Bleomycin and IL-1 beta-mediated pulmonary fibrosis is IL-17A dependent. J Exp Med 207, 535-552 (2010).
46. Longhi, M. S., et al. Impairment of CD4(+)CD25(+) regulatory T-cells in autoimmune liver disease. J Hepatol 41, 31-37 (2004).
47. Ge, J., et al. Implication of Th17 and Th1 cells in patients with chronic active hepatitis B. J Clin Immunol 30, 60-67 (2010).
48. Affo, S., et al. Transcriptome analysis identifies TNF superfamily receptors as potential therapeutic targets in alcoholic hepatitis. Gut (2012).
49. Bettelli, E., et al. Reciprocal developmental pathways for the generation of pathogenic effector TH17 and regulatory T cells. Nature 441, 235-238 (2006).
50. Mangan, P. R., et al. Transforming growth factor-beta induces development of the T(H)17 lineage. Nature 441, 231-234 (2006).
51. Weaver, C. T., Harrington, L. E., Mangan, P. R., Gavrieli, M. & Murphy, K. M. Th17: an effector CD4 T cell lineage with regulatory T cell ties. Immunity 24, 677-688 (2006).
52. Langrish, C. L., et al. IL-23 drives a pathogenic T cell population that induces autoimmune inflammation. J Exp Med 201, 233-240 (2005).
53. Murphy, C. A., et al. Divergent pro- and antiinflammatory roles for IL-23 and IL-12 in joint autoimmune inflammation. J Exp Med 198, 1951-1957 (2003).
54. Oppmann, B., et al. Novel p19 protein engages IL-12p40 to form a cytokine, IL-23, with biological activities similar as well as distinct from IL-12. Immunity 13, 715-725 (2000).
55. Cua, D. J., et al. Interleukin-23 rather than interleukin-12 is the critical cytokine for autoimmune inflammation of the brain. Nature 421, 744-748 (2003).
56. Fort, M. M., et al. IL-25 induces IL-4, IL-5, and IL-13 and Th2-associated pathologies in vivo. Immunity 15, 985-995 (2001).
57. Hurst, S. D., et al. New IL-17 family members promote Th1 or Th2 responses in the lung: in vivo function of the novel cytokine IL-25. J Immunol 169, 443-453 (2002).
58. Meng, F., et al. Interleukin-17 Signaling in Inflammatory Cells, Kupffer, and Hepatic Stellate Cells Exacerbates Liver Fibrosis in Mice. Gastroenterology (2012).
59. Parham, C., et al. A receptor for the heterodimeric cytokine IL-23 is composed of IL-12Rbeta1 and a novel cytokine receptor subunit, IL-23R. J Immunol 168, 5699-5708 (2002).
60. Zhang, J. Y., et al. Interleukin-17-producing CD4(+) T cells increase with severity of liver damage in patients with chronic hepatitis B. Hepatology 51, 81-91 (2010).
61. Gao, B. Hepatoprotective and anti-inflammatory cytokines in alcoholic liver disease. Journal of gastroenterology and hepatology 27 Suppl 2, 89-93 (2012).
62. Langowski, J. L., et al. IL-23 promotes tumour incidence and growth. Nature 442, 461-465 (2006).
63. Grivennikov, S. I., et al. Adenoma-linked barrier defects and microbial products drive IL-23/IL-17-mediated tumour growth. Nature 491, 254-258 (2012).
64. Li, J., et al. Interleukin 23 promotes hepatocellular carcinoma metastasis via NF-kappa B induced matrix metalloproteinase 9 expression. PLoS One 7, e46264 (2012).
65. Schaalan, M. F., Mohamed, W. A. & Amin, H. H. Vitamin D deficiency: correlation to interleukin-17, interleukin-23 and PIIINP in hepatitis C virus genotype 4. World J Gastroenterol 18, 3738-3744 (2012).
66. Xu, Y., et al. IL-23R polymorphisms, HBV infection, and risk of hepatocellular carcinoma in a high-risk Chinese population. J Gastroenterol (2012).
67. Petersen, B. C. & Lukacs, N. W. IL-17A and IL-25: therapeutic targets for allergic and exacerbated asthmatic disease. Future medicinal chemistry 4, 833-836 (2012).
68. Rickel, E. A., et al. Identification of functional roles for both IL-17RB and IL-17RA in mediating IL-25-induced activities. J Immunol 181, 4299-4310 (2008).
69. Lee, J., et al. IL-17E, a novel proinflammatory ligand for the IL-17 receptor homolog IL-17Rh1. J Biol Chem 276, 1660-1664 (2001).
70. Liu, C., et al. A CC′ loop decoy peptide blocks the interaction between Act1 and IL-17RA to attenuate IL-17- and IL-25-induced inflammation. Science signaling 4, ra72 (2011).
71. Liu, Y., Munker, S., Mullenbach, R. & Weng, H. L. IL-13 Signaling in Liver Fibrogenesis. Frontiers in immunology 3, 116 (2012).
72. Kleinschek, M. A., et al. IL-25 regulates Th17 function in autoimmune inflammation. J Exp Med 204, 161-170 (2007).
73. Zaph, C., et al. Commensal-dependent expression of IL-25 regulates the IL-23-IL-17 axis in the intestine. J Exp Med 205, 2191-2198 (2008).
74. Hall, A. O., Silver, J. S. & Hunter, C. A. The immunobiology of IL-27. Advances in immunology 115, 1-44 (2012).
75. Diveu, C., et al. IL-27 blocks RORc expression to inhibit lineage commitment of Th17 cells. J Immunol 182, 5748-5756 (2009).
76. Wirtz, S., et al. Protection from lethal septic peritonitis by neutralizing the biological function of interleukin 27. J Exp Med 203, 1875-1881 (2006).
77. Hibbert, L., Pflanz, S., De Waal Malefyt, R. & Kastelein, R. A. IL-27 and IFN-alpha signal via Stat1 and Stat3 and induce T-Bet and IL-12Rbeta2 in naive T cells. J Interferon Cytokine Res 23, 513-522 (2003).
78. Bertola A., M. S., Ki S., Wang H. Gao B. Mouse chronic plus binge ethanol feeding model (the NIAAA model). Nature Protocol, in press (2013).
79. Ueno, A., et al. Mouse intragastric infusion (iG) model. Nat Protoc 7, 771-781 (2012).
80. Kisseleva, T., et al. Myofibroblasts revert to an inactive phenotype during regression of liver fibrosis. Proc Natl Acad Sci USA 109, 9448-9453 (2012).
81. Thurman, R. G., et al. The role of gut-derived bacterial toxins and free radicals in alcohol-induced liver injury. Journal of gastroenterology and hepatology 13 Suppl, S39-50 (1998).
82. Brandon-Warner, E., Walling, T. L., Schrum, L. W. & McKillop, I. H. Chronic ethanol feeding accelerates hepatocellular carcinoma progression in a sex-dependent manner in a mouse model of hepatocarcinogenesis. Alcohol Clin Exp Res 36, 641-653 (2012).
83. Meng, F., et al. Alteration of interferon-alpha/beta receptors in chronic hepatitis B patients. J Clin Immunol 31, 521-532 (2011).
84. Hammerich, L., Heymann, F. & Tacke, F. Role of IL-17 and Th17 cells in liver diseases. Clin Dev Immunol 2011, 345803.
85. Jones, S. A., Sutton, C. E., Cua, D. & Mills, K. H. Therapeutic potential of targeting IL-17. Nat Immunol 13, 1022-1025 (2012).
86. Ye, Y., et al. Involvement of Th17 and Th1 effector responses in patients with Hepatitis B. J Clin Immunol 30, 546-555 (2010).
87. Villarino, A., et al. The IL-27R (WSX-1) is required to suppress T cell hyperactivity during infection. Immunity 19, 645-655 (2003).
88. Mann, B. S. & Chung, K. F. Blood neutrophil activation markers in severe asthma: lack of inhibition by prednisolone therapy. Respiratory research 7, 59 (2006).
89. Hampton, M. B., Kettle, A. J. & Winterbourn, C. C. Inside the neutrophil phagosome: oxidants, myeloperoxidase, and bacterial killing. Blood 92, 3007-3017 (1998).
90. Kisseleva, T., et al. Fibrocyte-like cells recruited to the spleen support innate and adaptive immune responses to acute injury or infection. Journal of molecular medicine 89, 997-1013 (2011).
91. Dubin, P. J., McAllister, F. & Kolls, J. K. Is cystic fibrosis a TH17 disease? Inflamm Res 56, 221-227 (2007).
92. Nakae, S., et al. Antigen-specific T cell sensitization is impaired in IL-17-deficient mice, causing suppression of allergic cellular and humoral responses. Immunity 17, 375-387 (2002).
93. Takeda, K., et al. Enhanced Th1 activity and development of chronic enterocolitis in mice devoid of Stat3 in macrophages and neutrophils. Immunity 10, 39-49 (1999).
94. Abdel Aziz, M. T., et al. Therapeutic potential of bone marrow-derived mesenchymal stem cells on experimental liver fibrosis. Clin Biochem 40, 893-899 (2007).
95. Clausen, B. E., Burkhardt, C., Reith, W., Renkawitz, R. & Forster, I. Conditional gene targeting in macrophages and granulocytes using LysMcre mice. Transgenic Res 8, 265-277 (1999).
96. Zhuo, L., et al. hGFAP-cre transgenic mice for manipulation of glial and neuronal function in vivo. Genesis 31, 85-94 (2001).
97. Funae, Y. & Imaoka, S. Simultaneous purification of multiple forms of rat liver microsomal cytochrome P-450 by high-performance liquid chromatography. Biochim Biophys Acta 842, 119-132 (1985).
98. Lu, Y., Wang, X. & Cederbaum, A. I. Lipopolysaccharide-induced liver injury in rats treated with the CYP2E1 inducer pyrazole. Am J Physiol Gastrointest Liver Physiol 289, G308-319 (2005).
99. Reinke, L. A. & Moyer, M. J. p-Nitrophenol hydroxylation. A microsomal oxidation which is highly inducible by ethanol. Drug metabolism and disposition: the biological fate of chemicals 13, 548-552 (1985).
100. Wu, D. & Cederbaum, A. Cytochrome P4502E1 sensitizes to tumor necrosis factor alpha-induced liver injury through activation of mitogen-activated protein kinases in mice. Hepatology 47, 1005-1017 (2008).
101. Tsukamoto, H., Mkrtchyan, H. & Dynnyk, A. Intragastric ethanol infusion model in rodents. Methods Mol Biol 447, 33-48 (2008).
102. Xiong, S., et al. Hepatic macrophage iron aggravates experimental alcoholic steatohepatitis. Am J Physiol Gastrointest Liver Physiol 295, G512-521 (2008).
103. Yan, A. W., et al. Enteric dysbiosis associated with a mouse model of alcoholic liver disease. Hepatology 53, 96-105 (2011).
104. Yan, A. W. & Schnabl, B. Bacterial translocation and changes in the intestinal microbiome associated with alcoholic liver disease. World journal of hepatology 4, 110-118 (2012).
105. Castano, A. P., et al. Serum amyloid P inhibits fibrosis through Fc gamma R-dependent monocyte-macrophage regulation in vivo. Sci Transl Med 1, 5ra13 (2009).
106. Grant, M. H., Melvin, M. A., Shaw, P., Melvin, W. T. & Burke, M. D. Studies on the maintenance of cytochromes P-450 and b5, monooxygenases and cytochrome reductases in primary cultures of rat hepatocytes. FEBS letters 190, 99-103 (1985).
107. Cederbaum, A. I. Introduction-serial review: alcohol, oxidative stress and cell injury. Free radical biology & medicine 31, 1524-1526 (2001).
108. Paik, Y. H., et al. Role of NADPH Oxidases in Liver Fibrosis. Antioxidants & redox signaling (2013).
109. Paik, Y. H. & Brenner, D. A. NADPH oxidase mediated oxidative stress in hepatic fibrogenesis. The Korean journal of hepatology 17, 251-257 (2011).
110. Hazra, S., et al. Peroxisome proliferator-activated receptor gamma induces a phenotypic switch from activated to quiescent hepatic stellate cells. J Biol Chem 279, 11392-11401 (2004).
111. She, H., Xiong, S., Hazra, S. & Tsukamoto, H. Adipogenic transcriptional regulation of hepatic stellate cells. J Biol Chem 280, 4959-4967 (2005).
112. Colmenero, J., et al. Hepatic expression of candidate genes in patients with alcoholic hepatitis: correlation with disease severity. Gastroenterology 132, 687-697 (2007).
113. Samavati, L., et al. STAT3 tyrosine phosphorylation is critical for interleukin 1 beta and interleukin-6 production in response to lipopolysaccharide and live bacteria. Molecular immunology 46, 1867-1877 (2009).
114. Mir, S. A., et al. Inhibition of signal transducer and activator of transcription 3 (STAT3) attenuates interleukin-6 (IL-6)-induced collagen synthesis and resultant hypertrophy in rat heart. J Biol Chem 287, 2666-2677 (2012).
115. Johnson, A. W., Kinzenbaw, D. A., Modrick, M. L. & Faraci, F. M. Small-Molecule Inhibitors of Signal Transducer and Activator of Transcription 3 Protect Against Angiotensin II-Induced Vascular Dysfunction and Hypertension. Hypertension 61, 437-442 (2013).
116. Bataller, R., Sancho-Bru, P., Gines, P. & Brenner, D. A. Liver fibrogenesis: a new role for the renin-angiotensin system. Antioxidants & redox signaling 7, 1346-1355 (2005).
117. Storch, J., Zhou, Y. X. & Lagakos, W. S. Metabolism of apical versus basolateral sn-2-monoacylglycerol and fatty acids in rodent small intestine. J Lipid Res 49, 1762-1769 (2008).
118. Liu, Y., et al. Knockdown of acyl-CoA:diacylglycerol acyltransferase 2 with antisense oligonucleotide reduces VLDL TG and ApoB secretion in mice. Biochim Biophys Acta 1781, 97-104 (2008).
119. Brandon-Warner, E., Schrum, L. W., Schmidt, C. M. & McKillop, I. H. Rodent models of alcoholic liver disease: of mice and men. Alcohol 46, 715-725 (2012).
120. Brandon-Warner, E., et al. Silibinin (Milk Thistle) potentiates ethanol-dependent hepatocellular carcinoma progression in male mice. Cancer letters 326, 88-95 (2012).
121. Numasaki, M., et al. Interleukin-17 promotes angiogenesis and tumor growth. Blood 101, 2620-2627 (2003).
122. Tartour, E., et al. Interleukin 17, a T-cell-derived cytokine, promotes tumorigenicity of human cervical tumors in nude mice. Cancer Res 59, 3698-3704 (1999).
123. Kuang, D. M., et al. Tumor-activated monocytes promote expansion of IL-17-producing CD8+ T cells in hepatocellular carcinoma patients. J Immunol 185, 1544-1549 (2010).
124. Zhang, J. P., et al. Increased intratumoral IL-17-producing cells correlate with poor survival in hepatocellular carcinoma patients. J Hepatol 50, 980-989 (2009).
125. Kryczek, I., et al. Cutting edge: Th17 and regulatory T cell dynamics and the regulation by IL-2 in the tumor microenvironment. J Immunol 178, 6730-6733 (2007).
126. Park, O., et al. In vivo consequences of liver-specific interleukin-22 expression in mice: Implications for human liver disease progression. Hepatology 54, 252-261 (2011).
127. Lai, D. Y. & Arcos, J. C. Minireview: dialkylnitrosamine bioactivation and carcinogenesis. Life sciences 27, 2149-2165 (1980).
128. He, G. & Karin, M. NF-kappaB and STAT3-key players in liver inflammation and cancer. Cell Res 21, 159-168 (2011).
129. Park, E. J., et al. Dietary and genetic obesity promote liver inflammation and tumorigenesis by enhancing IL-6 and TNF expression. Cell 140, 197-208 (2010).
130. He, G., et al. Hepatocyte IKKbeta/NF-kappaB inhibits tumor promotion and progression by preventing oxidative stress-driven STAT3 activation. Cancer Cell 17, 286-297 (2010).
131. Parganas, E., et al. Jak2 is essential for signaling through a variety of cytokine receptors. Cell 93, 385-395 (1998).
132. Kisseleva, T., Bhattacharya, S., Braunstein, J. & Schindler, C. W. Signaling through the JAK/STAT pathway, recent advances and future challenges. Gene 285, 1-24 (2002).
133. Hacker, H. & Karin, M. Regulation and function of IKK and IKK-related kinases. Science's STKE: signal transduction knowledge environment 2006, rel 3 (2006).
134. Karin, M. & Gallagher, E. TNFR signaling: ubiquitin-conjugated TRAFfic signals control stop-and-go for MAPK signaling complexes. Immunological reviews 228, 225-240 (2009).
135. Quintana, A., et al. Astrocyte-specific deficiency of interleukin-6 and its receptor reveal specific roles in survival, body weight and behavior. Brain, behavior, and immunity 27, 162-173 (2013).
136. Uyttenhove, C., Sommereyns, C., Theate, I., Michiels, T. & Van Snick, J. Anti-IL-17A autovaccination prevents clinical and histological manifestations of experimental autoimmune encephalomyelitis. Ann N Y Acad Sci 1110, 330-336 (2007).
137. Keller, T. L., et al. Halofuginone and other febrifugine derivatives inhibit prolyl-tRNA synthetase. Nature chemical biology 8, 311-317 (2012).
138. Sundrud, M. S., et al. Halofuginone inhibits TH17 cell differentiation by activating the amino acid starvation response. Science 324, 1334-1338 (2009).
139. Xu, T., et al. Ursolic acid suppresses interleukin-17 (IL-17) production by selectively antagonizing the function of RORgamma t protein. J Biol Chem 286, 22707-22710 (2011).
140. Solt, L. A., et al. Suppression of TH17 differentiation and autoimmunity by a synthetic ROR ligand. Nature 472, 491-494 (2011).
141. Pines, M. & Nagler, A. Halofuginone: a novel antifibrotic therapy. General pharmacology 30, 445-450 (1998).
142. McGaha, T., et al. Effect of halofuginone on the development of tight skin (TSK) syndrome. Autoimmunity 35, 277-282 (2002).
143. Elkin, M., et al. Inhibition of bladder carcinoma angiogenesis, stromal support, and tumor growth by halofuginone. Cancer Res 59, 4111-4118 (1999).

### INCORPORATION BY REFERENCE

Each and every publication and patent mentioned in the instant specification and drawings is herein incorporated by reference in its entirety for all purposes. Various modifications and variations of the described methods and system of the invention will be apparent to those skilled in the art without departing from the scope and spirit of the invention. Although the invention has been described below in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. A method for reducing one or more symptoms of one or more of steatohepatitis, liver fibrosis, and hepatocellular carcinoma (HCC) in a mammalian subject in need thereof, comprising administering to said mammalian subject a therapeutic amount of one or more compounds that reduce the level of one or more of
(a) interleukin 17 (IL-17),
(b) interleukin 23 (IL-23),
(c) signal transducer and activator of transcription 3 (Stat3), and
(d) Janus kinase 2 (Jak2),
in one or more of hepatic stellate cells (HSCs), liver resident Kupffer cells (KCs), immune cells, and inflammatory cells, wherein said therapeutic amount reduces said one or more symptoms.

2. The method of claim 1, wherein said steatohepatitis is alcoholic steatohepatitis.

3. The method of claim 1, wherein said liver fibrosis is alcoholic liver fibrosis.

4. The method of claim 1, wherein said liver fibrosis is cholestatic liver fibrosis.

5. The method of claim 1, wherein said liver fibrosis is hepatotoxic liver fibrosis.

6. The method of claim 1, wherein said one or more compounds is an antibody, or an antigen-binding fragment thereof, that specifically binds to said one or more of interleukin 17 (IL-17), interleukin 23 (IL-23), Stat3, and Jak2.

7. The method of claim 6, wherein said antibody is a monoclonal antibody.

8. The method of claim 7, wherein said monoclonal antibody is a human antibody.

9. The method of claim 8, wherein said human monoclonal antibody specifically binds to IL-17.

10. The method of claim 6, wherein said antibody is a humanized antibody.

11. A method for reducing one or more symptoms of one or more of steatohepatitis and liver fibrosis in a mammalian subject in need thereof, comprising administering to said mammalian subject a therapeutic amount of one or more compounds that increase the level of one or more of
(a) interleukin 25 (IL-25), and
(b) interleukin 27 (IL-27),
in one or more of immune cells, inflammatory cells, and liver resident fibrogenic myofibroblasts (Hepatic stellate cells), wherein said therapeutic amount reduces said one or more symptoms.

12. A method for reducing one or more symptoms of one or more of steatohepatitis and liver fibrosis in a mammalian subject in need thereof, comprising administering to said mammalian subject a therapeutic amount of one or more of
(a) interleukin 25 (IL-25), and
(b) interleukin 27 (IL-27),
wherein said therapeutic amount reduces said one or more symptoms.


SEQUENCE LISTING (48 sequences)

SEQ ID NO: 1 - DNA, 20 bp, Artificial Sequence
  Synthetic
cttgactctgcagctcagcc

SEQ ID NO: 2 - DNA, 18 bp, Artificial Sequence
  Synthetic
atggctgcttctgctgct

SEQ ID NO: 3 - DNA, 20 bp, Artificial Sequence
  Synthetic
cctgctcctcagagacatcc

SEQ ID NO: 4 - DNA, 20 bp, Artificial Sequence
  Synthetic
atctggtcctacacgaagcc

SEQ ID NO: 5 - DNA, 20 bp, Artificial Sequence
  Synthetic
tgagcttcccagatcacaga

SEQ ID NO: 6 - DNA, 20 bp, Artificial Sequence
  Synthetic
tccagaaggccctcagacta

SEQ ID NO: 7 - DNA, 20 bp, Artificial Sequence
  Synthetic
ttgatgcagcctgagtgtct

SEQ ID NO: 8 - DNA, 20 bp, Artificial Sequence
  Synthetic
aattccagaaccgctccagt

SEQ ID NO: 9 - DNA, 23 bp, Artificial Sequence
  Synthetic
accagaggaaattttcaataggc

SEQ ID NO: 10 - DNA, 20 bp, Artificial Sequence
  Synthetic
tgatgcacttgcagaaaaca

SEQ ID NO: 11 - DNA, 20 bp, Artificial Sequence
  Synthetic
ggtcaaaggtttggaagcag

SEQ ID NO: 12 - DNA, 20 bp, Artificial Sequence
  Synthetic
tgtgaaatgccaccttttga

SEQ ID NO: 13 - DNA, 20 bp, Artificial Sequence
  Synthetic
caacccaggtccttcctaaa

SEQ ID NO: 14 - DNA, 20 bp, Artificial Sequence
  Synthetic
ggagagccctggataccaac

SEQ ID NO: 15 - DNA, 20 bp, Artificial Sequence
  Synthetic
agggtctgggccatagaact

SEQ ID NO: 16 - DNA, 21 bp, Artificial Sequence
  Synthetic
ccaccacgctcttctgtctac

SEQ ID NO: 17 - DNA, 20 bp, Artificial Sequence
  Synthetic
agccttggctgagtggtaga

SEQ ID NO: 18 - DNA, 20 bp, Artificial Sequence
  Synthetic
cgatgatgaacgatggacag

SEQ ID NO: 19 - DNA, 20 bp, Artificial Sequence
  Synthetic
taggccattgtgtatgcagc

SEQ ID NO: 20 - DNA, 21 bp, Artificial Sequence
  Synthetic
acatgttcagctttgtggacc

SEQ ID NO: 21 - DNA, 20 bp, Artificial Sequence
  Synthetic
aggtggtctcgttgattcgt

SEQ ID NO: 22 - DNA, 20 bp, Artificial Sequence
  Synthetic
gtaaggcctgtagctgtgcc

SEQ ID NO: 23 - DNA, 20 bp, Artificial Sequence
  Synthetic
gttcagtggtgcctctgtca

SEQ ID NO: 24 - DNA, 20 bp, Artificial Sequence
  Synthetic
actgggacgacatggaaaag

SEQ ID NO: 25 - DNA, 21 bp, Artificial Sequence
  Synthetic
agtccctgccctttgtacaca

SEQ ID NO: 26 - DNA, 19 bp, Artificial Sequence
  Synthetic
cgatccgagggcctcacta

SEQ ID NO: 27 - DNA, 20 bp, Artificial Sequence
  Synthetic
gtcaggggtggttattgcat

SEQ ID NO: 28 - DNA, 20 bp, Artificial Sequence
  Synthetic
agtgaggaacaagccagagc

SEQ ID NO: 29 - DNA, 20 bp, Artificial Sequence
  Synthetic
aaatttggggtggaaaggtt

SEQ ID NO: 30 - DNA, 21 bp, Artificial Sequence
  Synthetic
tcctgatttctgcagctctgt

SEQ ID NO: 31 - DNA, 22 bp, Artificial Sequence
  Synthetic
gccctggacaccaactattgct

SEQ ID NO: 32 - DNA, 22 bp, Artificial Sequence
  Synthetic
aggctccaaatgtaggggcagg

SEQ ID NO: 33 - DNA, 20 bp, Artificial Sequence
  Synthetic
cacacgtctcggtcatggta

SEQ ID NO: 34 - DNA, 20 bp, Artificial Sequence
  Synthetic
aagaggaaggccaagtcgag

SEQ ID NO: 35 - DNA, 20 bp, Artificial Sequence
  Synthetic
acaggttccagccctacatg

SEQ ID NO: 36 - DNA, 18 bp, Artificial Sequence
  Synthetic
gtcgtcaccgctgatgtg

SEQ ID NO: 37 - DNA, 20 bp, Artificial Sequence
  Synthetic
ccaggtggttgcattcttgg

SEQ ID NO: 38 - DNA, 20 bp, Artificial Sequence
  Synthetic
tggctgtaggtgtgggttcc

SEQ ID NO: 39 - DNA, 20 bp, Artificial Sequence
  Synthetic
gaacgcacatgcaccagcgg

SEQ ID NO: 40 - DNA, 24 bp, Artificial Sequence
  Synthetic
tgcaagcagaactggctgttgtcc

SEQ ID NO: 41 - DNA, 20 bp, Artificial Sequence
  Synthetic
aggtggtctcgttgattcgt

SEQ ID NO: 42 - DNA, 20 bp, Artificial Sequence
  Synthetic
gtaaggcctgtagctgtgcc

SEQ ID NO: 43 - DNA, 20 bp, Artificial Sequence
  Synthetic
ccagagccattgtcacacac

SEQ ID NO: 44 - DNA, 18 bp, Artificial Sequence
  Synthetic
cagccaagcactgtcagg

SEQ ID NO: 45 - DNA, 20 bp, Artificial Sequence
  Synthetic
cactttgcctcccagatcac

SEQ ID NO: 46 - DNA, 20 bp, Artificial Sequence
  Synthetic
accaatcccaaaaggtcctc

SEQ ID NO: 47 - DNA, 20 bp, Artificial Sequence
  Synthetic
cctcactcagaaaggcaagc

SEQ ID NO: 48 - DNA, 20 bp, Artificial Sequence
  Synthetic
agcgcaacatgacagtgaag